(19) United States
(12) Patent Application Publication
(10) Pub. No.: US 2022/0313681 A1
(43) Pub. Date: Oct. 6, 2022

(54) NOVEL CELL METABOLISM MODULATING COMPOUNDS AND USES THEREOF FOR THE TREATMENT OF VIRAL DISEASES

(71) Applicant: CRESCENTA BIOSCIENCES, Union, NJ (US)

(72) Inventors: Emre KOYUNCU, Irvine, CA (US); Hahn KIM, Princeton, NJ (US); Gokhan Hotamisligil, Weston, MA (US)

(73) Assignee: CRESCENTA BIOSCIENCES, Union, NJ (US)

(21) Appl. No.: 17/742,899

(22) Filed: May 12, 2022

Related U.S. Application Data
(63) Continuation of application No. 17/566,692, filed on Dec. 31, 2021.
(60) Provisional application No. 63/048,619, filed on Jul. 6, 2020.

(51) Int. Cl. A61K 31/47; A61K 31/506; A61P 31/12

(52) U.S. Cl. CPC A61K 31/47; A61K 31/506; A61P 31/12

(57) ABSTRACT

A class of compounds that bind to FABP4 and inhibit viral replication in cells, as well as pharmaceutical compositions comprising the class of compounds, in combination with a pharmaceutically acceptable diluent or carrier, and optionally, further in combination with an additional therapeutically active agent, and the use of these compounds in medicine and for the preparation of a medicament in the treatment of viral diseases.

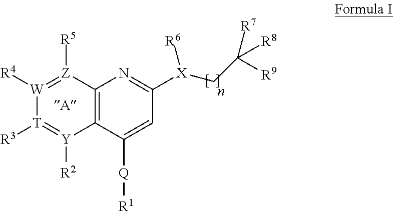

### CROSS-REFERENCE TO RELATED APPLICATIONS SECTION

This Application is a U.S. Non-Provisional Patent Application and continuation application that claims priority to U.S. Non-Provisional patent application Ser. No. 17/566,692 filed on Dec. 31, 2021, which claims priority to U.S. Provisional Patent Application S/N 63/048,619 filed on Jul. 10, 2020, the entire contents of which are hereby incorporated by reference in their entirety.

### FIELD OF THE EMBODIMENTS

The field of the embodiments of the present invention relate to antiviral therapies for treatment or prophylaxis of diseases caused by coronavirus infections. Compounds in this invention particularly interact with fatty acid binding protein 4 (FABP4) and inhibit the growth of coronaviruses in cells.

### BACKGROUND OF THE EMBODIMENTS

FABPs are a family of proteins that reversibly bind free fatty acids and other lipid molecules and facilitate their transport in cells. To date, nine different FABP isoforms have been identified in mammals. FABP isoforms display differential expression patterns in different tissues. Fatty acid binding protein 4 (FABP4), also often referred to as aP2 in literature, is primarily expressed in adipocytes and macrophages, and mediates key metabolic and inflammatory pathways in these cells such as lipid storage and degradation, signaling and eicosanoid production. In addition, FABP4 is also secreted to plasma and has been proposed to act as an adipose-derived factor regulating systemic glucose homeostasis.

The present invention relates to the pharmaceutical industry and medicines and presents a new therapy for the treatment and prevention of acute viral infections, particularly acute respiratory viral infections and more particularly to therapy for coronavirus and severe acute respiratory syndrome (SARS) diseases, comprising the administration of a therapeutically effective dose of a compound described herein to a subject in need thereof.

Acute respiratory viral infections are a group of infectious diseases of viral etiology and characterized by damage to the respiratory tract and exhibiting common clinical symptoms. The successes achieved by medicine in the field of infectious pathology have contributed to the development and improvement of methods for the prevention and treatment of diseases, but acute respiratory viral infections continue to be a serious public health problem due to extremely high rates of incidence and the occurrence of epidemics and pandemics.

Acute respiratory viral infections are polyetiological. Currently, more than 200 relevant pathogens are known with rhinoviruses, coronaviruses, and influenza viruses frequently seen causing a high outbreak in the autumn and winter. Other viruses causing acute respiratory viral infections at relatively higher rates include respiratory syncytial viruses (RSVs), parainfluenza, adenoviruses, Human metapneumovirus (HMPV), Human Bocavirus (HBoV) along with unidentified viruses responsible for the remainder of acute infections of the respiratory tract of viral and unspecified etiology.

Of recent concern, SARS, a form of acute respiratory viral infection, presents as one of the leading severe diseases active in children and adults, resulting in temporary disability and significant mortality. The high incidence of SARS is associated with significant economic losses due to indirect costs associated with disability and compounded by societal and economic damage from governmentally imposed lockdowns instituted to prevent viral spread. Most seasonal morbidity is affected by children, the elderly, people with concomitant diseases (various immunodeficiencies, diseases of the lungs, cardiovascular system, liver, kidneys, diabetes, etc.).

Acute respiratory diseases of coronavirus etiology can be severe and cause extensive damage to lung tissue as well as other organs. Common human coronaviruses cause varying levels of acute respiratory infections. Viruses HCoV-OC43, and HCoV-HKU1, HCoV-229E, and HCoV-NL63 cause common colds and self-limiting upper respiratory infections in immunocompetent individuals. In immunocompromised subjects and the elderly, lower respiratory tract infections can occur. Other coronaviruses can cause more severe form of acute respiratory disease and include SARS-CoV, SARS-CoV-2, and MERS-CoV. These coronaviruses can cause epidemics and pandemics with variable clinical severity featuring respiratory and extra-respiratory manifestations.

The COVID-19 pandemic caused by SARS-CoV-2 dramatically demonstrated that there is a great unmet need for effective therapeutic agents that can broadly treat or prevent coronavirus infections. This highlighted the importance of host factors, independent of the viral proteins, that are intimately related to the virus life cycle and also the disease severity and mortality associated with viral infections.

The invention and embodiments thereof disclosed herein describes a novel class of antiviral agents that interact with fatty acid binding protein 4 (FABP4) and inhibit viral replication in human cells. FABP4 is a key mediator of fatty acid metabolism and inflammation, two pathways that have been implicated in the life cycle of many viral pathogens. Furthermore, FABP4 has been particularly associated with the development of a number of metabolic conditions such as diabetes, cardiovascular disease, and airway inflammation that are known to confer susceptibility to coronavirus, influenza virus and certain other virus infections. In view of the present discovery that compounds that target FABP4 also inhibit viral replication in cells, the compounds described in this invention present a universally applicable treatment for disease caused by known viruses and variants that emerge in future.

### SUMMARY OF THE EMBODIMENTS

The invention is a method for treating a viral disease in a subject comprising administering to said subject a therapeutically effective dose of one or more of the compounds described in Formula (I):

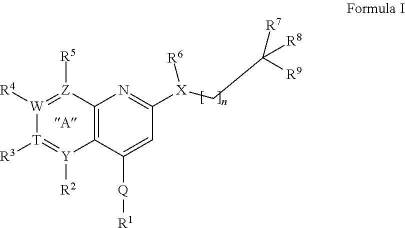

or pharmaceutically acceptable salts or esters thereof, wherein:

wherein each of R¹ and R⁶-R⁹ are independently —H, —CN, —COOH, —CONH₂, B(ORₐ)₂, an acid isostere, a halo, Cₙ alkyl, Cₙ alkenyl, Cₙ alkynyl, Cₙ aryl, Cₙ aminoalkyl, Cₙ haloalkyl, Cₙ heteroaryl, Cₙ cycloalkyl, or Cₙ heterocycloalkyl,

wherein Rₐ of the B(ORₐ)₂ is H or an alkyl,

wherein B of the B(ORₐ)₂ is boron,

wherein n of the Cₙ is 1-10,

wherein each of R²-R⁵ are independently —H, —CN, —COOH, —COOMe, —CONH₂, B(ORₐ)₂, the acid isostere, the halo, —CONHOH, —NH—SO₂—C_{ι}—C₆-alkyl, —NHSO₂Ar, the Cₙ alkyl, the Cₙ alkyl, the Cₙ alkenyl, the Cₙ alkynyl, the Cₙ aryl, the Cₙ aminoalkyl, the Cₙ haloalkyl, the Cₙ heteroaryl, the Cₙ cycloalkyl, or the Cₙ heterocycloalkyl, wherein the Ar of —NHSO₂Ar is selected from the group consisting of: phenyl, naphthyl, pyrrole, imidazole, thiophene, furan, thiazole, isothiazole, thiadiazole, oxazole, isoxazole, oxadiazole, pyridine, pyrazine, pyrimidine, pyridazine, pyrazole, triazole, tetrazole, chroman, isochroman, quinoline, quinoxaline, isoquinoline, phthalazine, cinnoline, quinazoline, indole, isoindole, indoline, isoindoline, benzothiophene, benzofuran, isobenzofuran, benzoxazole, 2,1,3-benzoxadiazole, benzothiazole, 2,1,3-benzothiazole, 2,1,3-benzoselenadiazole, benzimidazole, indazole, benzodioxane, indane, 1,2,3,4-tetrahydroquinoline, 3,4-dihydro-2H-1,4-benzoxazine, 1,5- naphthyridine, 1,8-naphthyridine, acridine, phenazine, and xanthene,

wherein each of the Cₙ alkyl, the Cₙ alkenyl, the Cₙ alkynyl, the Cₙ aryl, the C. aminoalkyl, the Cₙ haloalkyl, the Cₙ heteroaryl, the Cₙ cycloalkyl, and the Cₙ heterocycloalkyl is unsubstituted or substituted with a quantity of substituents being between 1-5,
wherein each of the substituents is the same or different,
wherein each of the substituents is selected from the group consisting of H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, —C(O)alkyl, and —C_{q}—U—C_{q},

wherein each q of —C_{q}—U—C_{q} is independently 0 to 10,

wherein the U of —C_{q}—U—C_{q} is any one of aryl, heteroaryl, cycloalkyl, heterocycloalkyl, O, S, SO₂, or N(R₁)(R₁),

wherein each R₁ of N(R₁)(R₁) is independently hydrogen, the Cₙ alkyl, the Cₙ alkenyl, the

Cₙ alkynyl, the Cₙ aryl, the Cₙ aminoalkyl, the Cₙ haloalkyl, the Cₙ heteroaryl, the Cₙ cycloalkyl, or the Cₙ heterocycloalkyl,

wherein each R₁ of N(R₁)(R₁) is unsubstituted or substituted with 1, 2, 3, 4 or 5 substituents which can be the same or different and are independently selected from the group consisting of: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, and —C(O)alkyl,

wherein Q is a bond or O,

wherein X is C, N, O, or S such that R⁶ is not present if X is O or S,

wherein “A” is a saturated or unsaturated ring depicted by

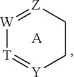

wherein Y, T, W, and Z are independently a bond, C, N, O, an alkyl having 1 to 4 carbon atoms, or an alkenyl having 1 to 4 carbon atoms, and

wherein n is 0, 1, 2, or 3.

In one embodiment, the invention comprises the compound of Formula I where at least one of R¹ and R², R² and R³, R³ and R⁴, R⁴ and R⁵, R⁶ and R⁷, R⁷ and R⁸, R⁸ and R⁹ are bonded forming a fused heteroaryl or fused heterocycloalkyl.

In a further embodiment, the invention is a method for treating a viral disease in a subject comprising administering to said subject a therapeutically effective dose of one or more of the compounds described in Formula II:

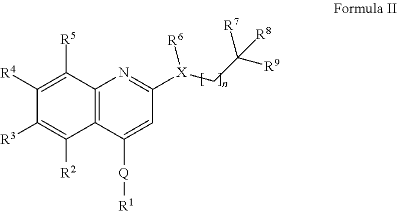

or pharmaceutically acceptable salts or esters thereof wherein:

wherein R¹ and R⁶-R⁹ is independently —H, —CN, —COOH, —CONH₂, B(ORₐ)₂, an acid isostere, a halo, Cₙ alkyl, Cₙ alkenyl, Cₙ alkynyl, Cₙ aryl, Cₙ aminoalkyl, Cₙ haloalkyl, Cₙ heteroaryl, Cₙ cycloalkyl, or Cₙ heterocycloalkyl,

wherein Rₐ of the B(ORₐ)₂ is H or an alkyl,

wherein B of the B(ORₐ)₂ is boron,

wherein n of the C. is 1 — 10,

wherein each of R²-R⁵ are independently —H, —CN, —COOH, —COOMe, —CONH₂, B(ORₐ)₂, the acid isostere, the halo, —CONHOH, —NH—SO₂—C_{ι}—C₆-alkyl, —NHSO₂Ar, the Cₙ alkyl, the Cₙ alkyl, the Cₙ alkenyl, the Cₙ alkynyl, the Cₙ aryl, the Cₙ aminoalkyl, the Cₙ haloalkyl, the Cₙ heteroaryl, the Cₙ cycloalkyl, or the Cₙ heterocycloalkyl,

wherein the Ar of —NHSO₂Ar is selected from the group consisting of: phenyl, naphthyl, pyrrole, imidazole, thiophene, furan, thiazole, isothiazole, thiadiazole, oxazole, isoxazole, oxadiazole, pyridine, pyrazine, pyrimidine, pyridazine, pyrazole, triazole, tetrazole, chroman, isochroman, quinoline, quinoxaline, isoquinoline, phthalazine, cinnoline, quinazoline, indole, isoindole, indoline, isoindoline, benzothiophene, benzofuran, isobenzofuran, benzoxazole, 2,1,3-benzoxadiazole, benzothiazole, 2,1,3-benzothiazole, 2,1,3-benzoselenadiazole, benzimidazole, indazole, benzodioxane, indane, 1,2,3,4-tetrahydroquinoline, 3,4-dihydro-2H-1,4-benzoxazine, 1,5- naphthyridine, 1,8-naphthyridine, acridine, phenazine, and xanthene,

wherein each of the Cₙ alkyl, the Cₙ alkenyl, the Cₙ alkynyl, the Cₙ aryl, the C. aminoalkyl, the Cₙ haloalkyl, the Cₙ heteroaryl, the Cₙ cycloalkyl, or the Cₙ heterocycloalkyl is unsubstituted or substituted with a quantity of substituents being between 1-5,

wherein each of the substituents is the same or different,

wherein each of the substituents is selected from the group consisting of: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, —C(O)alkyl, and —C_{q}—U—C_{q},

wherein each q of —C_{q}—U—C_{q} is independently 0 to 10,

wherein the U of —C_{q}—U—C_{q} is any one of aryl, heteroaryl, cycloalkyl, heterocycloalkyl, O, S, SO₂, or N(R₁)(R₁),

wherein each R₁ of N(R₁)(R₁) is independently hydrogen, the Cₙ alkyl, the Cₙ alkenyl, the Cₙ alkynyl, the Cₙ aryl, the Cₙ aminoalkyl, the Cₙ haloalkyl, the Cₙ heteroaryl, the Cₙ cycloalkyl, or the Cₙ heterocycloalkyl,

wherein each R₁ of N(R₁)(R₁) is unsubstituted or substituted with 1, 2, 3, 4 or 5 substituents which can be the same or different and are independently selected from the group consisting of: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, and —C(O)alkyl,

wherein Q is a bond or O,

wherein X is C, N, O, or S such that R⁶ is not present if X is O or S,

wherein X is C, N, O, or S, and

wherein n is 0, 1, 2, or 3.

In some embodiments, the invention includes compounds of Formula II, wherein a heterocycloalkyl group is formed by bonding two of R⁷, R⁸, or R⁹ to form:

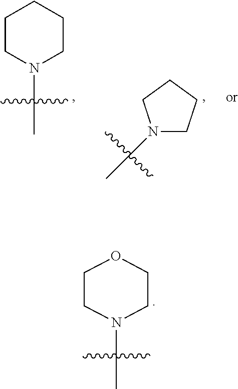 [SMILES: *N1CCCCC1 ; *N1CCCC1 ; *N1CCOCC1]

Additional embodiments are a method for treating a viral disease in a subject comprising administering to said subject a therapeutically effective dose of one or more of the compounds described in Formula III:

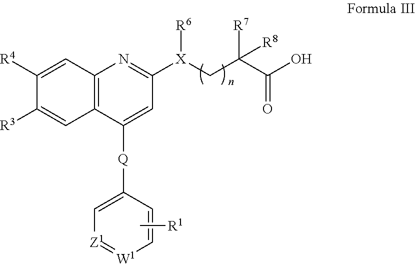

or pharmaceutically acceptable salts or esters thereof, wherein:

wherein R¹ is selected from the group consisting of: —CN, alkyl, —H, halo, ²H, amino, alkoxy, aminoalkyl, (amino)alkoxy, alkenyl, alkynyl, alkoxy, hydroxy, alkylhydroxy, aryloxy, alkyl(aryl), (alkoxyalkyl)amino, aryl, aryl(halo), heteroaryl, hydroxyl-alkyl, hydroxyl-aryl, (aryl)alkyl, C(O)OH, —S(O)₂-alkyl, —S(O)₂-aryl, —C(O)alkyl, and C(O)NH₂,

wherein each of R³ and R⁴ are independently —H, the halo, Cₙ alkyl, Cₙ alkyl, Cₙ alkenyl, Cₙ alkynyl, Cₙ aryl, Cₙ aminoalkyl, Cₙ haloalkyl, Cₙ heteroaryl, Cₙ cycloalkyl, Cₙ heterocycloalkyl, and —C_{q}—U—C_{q},

wherein n of Cₙ is 1-10,

wherein each q of —C_{q}—U—C_{q} is independently 0 to 10,

wherein the U of —C_{q}—U—C_{q} is any one of O, S, SO₂, or N(R₁)(R₁),

wherein each R₁ of N(R₁)(R₁) are independently hydrogen,

wherein each of the Cₙ alkyl, the Cₙ alkenyl, the Cₙ alkynyl, the Cₙ aryl, the Cₙ aminoalkyl, the Cn haloalkyl, the Cn heteroaryl, the Cₙ cycloalkyl, and the Cn heterocycloalkyl are unsubstituted or substituted with a quantity of substituents being between 1-5,

wherein each of the substituents are the same or different,

wherein each of the substituents is selected from the group consisting of: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, and —C(O)alkyl,

wherein R⁶ is independently H or alkyl,

wherein each of R⁷ and R⁸ are independently hydrogen, ²H, fluoro or alkyl,

wherein R⁶ and R⁷ are bonded with an adjoining R group to form a fused group,

wherein the fused group is selected from the group consisting of: a fused cycloalkyl, a fused heterocycloalkyl, a fused aryl, and a fused heteroaryl ring,

wherein the fused group comprises between 4 to 10 carbon atoms,

wherein n of Formula III is 0 or 1,

wherein Q is a bond or O,

wherein X is C, N, O, or S such that R⁶ is not present if X is O or S, and

wherein Z¹ or W¹ are independently C, N, O, or S.

Another embodiment of the invention includes compounds of Formula III wherein X is N.

An embodiment the invention includes compounds of Formula III wherein R⁶ is hydrogen and X is N.

A further embodiment the invention includes compounds of Formula III wherein R³ is alkyl and R¹ is cyano.

An additional embodiment the invention includes compounds of Formula III wherein X is N and n=0.

An embodiment the invention includes compounds of Formula III wherein X is N and n=1.

It is an object of the present invention to utilize the compounds described herein in the treatment of viral disorders by acting on the fatty acid binding protein (FABP4).

Yet another object of the present invention is a pharmaceutical composition comprising a compounds herein as active ingredient, in combination with a pharmaceutically acceptable diluent or carrier for use in the treatment of viral disorders by acting on FABP4. Here, the pharmaceutical composition can further comprise an additional therapeutically active agent.

Yet another object of the present invention is a method for the treatment of viral disorders by acting on the FABP4, which comprises administering to a subject in need of such treatment (preferably, a human) an effective amount of the compounds herein, including, optionally, the co-administration with other therapeutic agents, either as a single (or multiple) dosing, and either simultaneously or sequentially.

Yet another object of the present invention is the use of the compounds herein for the manufacture of a medicament for use in the treatment of viral disorders by acting on the fatty acid binding protein FABP4.

Any compound disclosed herein which is a pure optical isomer.

Any compound disclosed herein which is the (+)-isomer.

Examples of such disorders include common cold, SARS, and COVID-19.

Other features and advantages of the invention will be apparent from the detailed description and the claims.

### BRIEF DESCRIPTION OF THE DRAWINGS

FIG. 1 depicts a graphical chart showing a virus yield obtained from cells treated with corresponding compounds and vehicle control (DMSO), according to at least some embodiments disclosed herein.

### DESCRIPTION OF THE PREFERRED EMBODIMENTS

Reference will now be made in detail to each embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be.

### Definitions

As used herein, the term “acid isostere” includes, but is not limited to, the following functional groups, where R is H or alkyl.

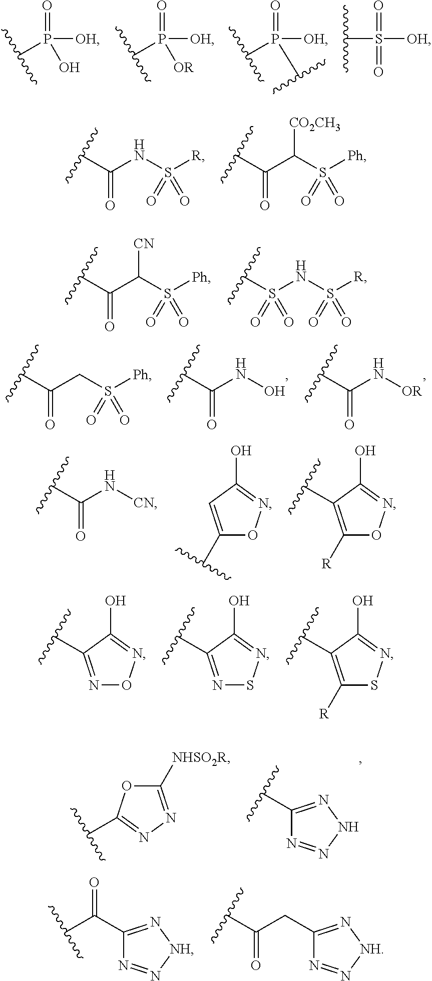

The term “alkyl” refers to a saturated, straight- or branched-chain hydrocarbon group having from 1 to 20 carbon atoms. Representative alkyl groups include, but are not limited to, methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, n-pentyl, isopentyl, neopentyl, n-hexyl, and the like, and longer alkyl groups, such as heptyl, octyl, and the like. Moreover, the number of carbons on an alkyl chain can be defined in association with the C atom such as C₁₋₁₀, where C₁₋₁₀ is a carbon chain having 1 to 10 carbon atoms.

The term “alkoxy” as used herein includes —O-(alkyl), wherein alkyl is defined above.

The term “amino” as used herein refers to an —NH₂ group.

The term “aryl” means a mono-, bi-, or tricyclic aromatic group, wherein all rings of the group are aromatic and all ring atoms are carbon atoms. For bi- or tricyclic systems, the individual aromatic rings are fused to one another. Examples of aryl groups are 6 and 10 membered aryls. Further examples of aryl groups include, but are not limited to, phenyl, naphthalene, and anthracene.

The term “cyano” as used herein means a substituent having a carbon atom joined to a nitrogen atom by a triple bond.

The term “deuterium” as used herein means a stable isotope of hydrogen having one proton and one neutron.

The term “halo” represents chloro, fluoro, bromo, or iodo. In some embodiments, halo is chloro, fluoro, or bromo. The term “halogen” as used herein refers to fluorine, chlorine, bromine, or iodine.

The term “hydroxy” means an —OH group. The term “oxo” means an ═O group and may be attached to a carbon atom or a sulfur atom.

The term “N-oxide” refers to the oxidized form of a nitrogen atom.

The term “cycloalkyl” refers to a saturated or partially saturated, monocyclic, fused polycyclic, bridged polycyclic, or spiro polycyclic carbocycle having from 3 to 15 carbon ring atoms. A non limiting category of cycloalkyl groups are saturated or partially saturated, monocyclic carbocycles having from 3 to 6 carbon atoms. Illustrative examples of cycloalkyl groups include, but are not limited to, the following moieties:

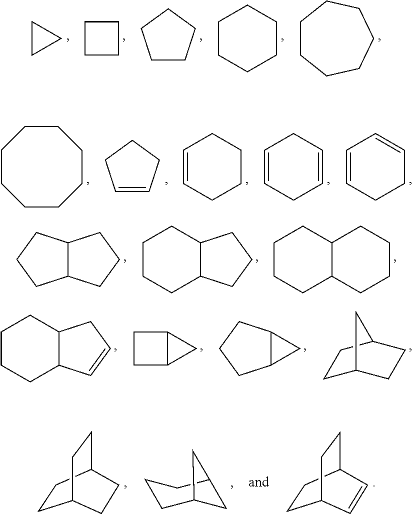

The term “heterocycloalkyl” as used herein refers to a monocyclic, or fused, bridged, or spiro polycyclic ring structure that is saturated or partially saturated and has from three to 12 ring atoms selected from carbon atoms and up to three heteroatoms selected from nitrogen, oxygen, and sulfur. The ring structure may optionally contain up to two oxo groups on carbon or sulfur ring members, or an N-oxide. Illustrative heterocycloalkyl entities include, but are not limited to:

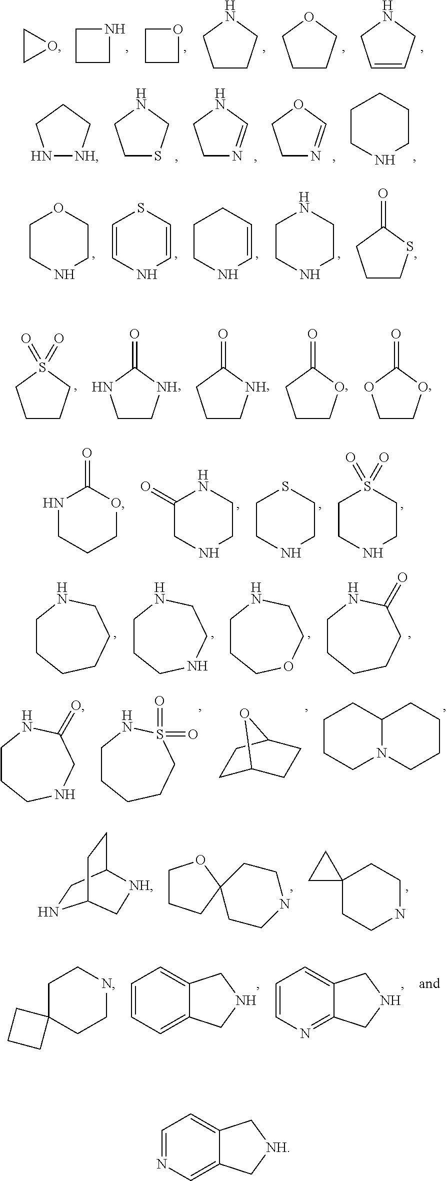

The term “heteroaryl” refers to a monocyclic, or fused polycyclic, aromatic heterocycle having from three to 15 ring atoms that are selected from carbon, oxygen, nitrogen, and sulfur. Suitable heteroaryl groups do not include ring systems that must be charged to be aromatic, such as pyrylium. Suitable 5-membered heteroaryl rings (as a monocyclic heteroaryl or as part of a polycyclic heteroaryl) have one oxygen, sulfur, or nitrogen ring atom, or one nitrogen plus one oxygen or sulfur, or 2, 3, or 4 nitrogen ring atoms. Suitable 6-membered heteroaryl rings (as a monocyclic heteroaryl or as part of a polycyclic heteroaryl) have 1, 2, or 3 nitrogen ring atoms. Examples of heteroaryl groups include, but are not limited to, pyridinyl, imidazolyl, imidazopyridinyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, triazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl.

The term “fused heteroaryl” refers to a heteroaryl as defined above, having two constituent aromatic rings, wherein the two rings are fused to one another and at least one of the rings is a heteroaryl as defined above. Fused heteroaryls include fused heteroaryl groups comprising 1, 2, 3, or 4 heteroatom ring atoms selected from O, N or S. In certain embodiments, wherein the heteroatom is N it can be an N-oxide. Fused heteroaryls also include 8-, 9-, or 10-membered fused heteroaryl groups. Fused heteroaryls also include 8-, 9-, or 10-membered fused heteroaryl groups that have 1, 2, 3, or 4 heteroatom ring atoms selected from O, N or S. Illustrative examples of fused heteroaryls include, but are not limited to:

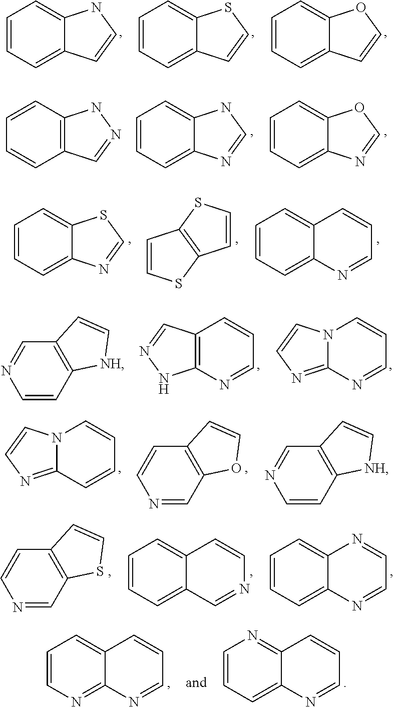

Those skilled in the art will recognize that the species of heteroaryl, cycloalkyl, and heterocycloalkyl groups listed or illustrated above are not exhaustive, and that additional species within the scope of these defined terms may also be selected.

As used herein, the term “substituted” means that the specified group or moiety bears one or more suitable substituents. As used herein, the term “unsubstituted” means that the specified group bears no substituents. As used herein, the term “optionally substituted” means that the specified group is unsubstituted or substituted by the specified number of substituents. Where the term “substituted” is used to describe a structural system, the substitution is meant to occur at any valency-allowed position on the system.

Any atom that is represented herein with an unsatisfied valence is assumed to have the sufficient number of hydrogen atoms to satisfy the atom's valence.

When any variable (e.g., alkyl, R^{a}, R¹, etc.) appears in more than one place in any formula or description provided herein, the definition of that variable on each occurrence is independent of its definition at every other occurrence.

Numerical ranges, as used herein, are intended to include sequential whole numbers. For example, a range expressed as “from 0 to 4” or “0-4” includes 0, 1, 2, 3 and 4.

When a multifunctional moiety is shown, the point of attachment to the remainder of the formula can be at any point on the multifunctional moiety. In some embodiments, the point of attachment is indicated by a line or hyphen. For example, aryloxy- refers to a moiety in which an oxygen atom is the point of attachment to the core molecule while aryl is attached to the oxygen atom.

As used herein, the term “subject” encompasses mammals and non-mammals. Examples of mammals include, but are not limited to, any member of the Mammalian class: humans; non-human primates such as chimpanzees, and other apes and monkey species; farm animals such as cattle, horses, sheep, goats, swine; domestic animals such as rabbits, dogs, and cats; and laboratory animals including rodents, such as rats, mice and guinea pigs, and the like. Examples of non-mammals include, but are not limited to, birds, fish and the like. In one embodiment of the present invention, the mammal is a human.

The term “patient” includes both human and animals.

The terms “effective amount” or “therapeutically effective amount” refer to a sufficient amount of the agent to provide the desired biological result. That result can be reduction and/or alleviation of the signs, symptoms, or causes of a disease or medical condition, or any other desired alteration of a biological system. For example, an “effective amount” for therapeutic use is the amount of a compound, or of a composition comprising the compound, that is required to provide a clinically relevant change in a disease state, symptom, or medical condition. An appropriate “effective” amount in any individual case may be determined by one of ordinary skill in the art using routine experimentation. Thus, the expression “effective amount” generally refers to the quantity for which the active substance has a therapeutically desired effect.

As used herein, the terms “treat” or “treatment” encompass both “preventative” and “curative” treatment. “Preventative” treatment is meant to indicate a postponement of development of a disease, a symptom of a disease, or medical condition, suppressing symptoms that may appear, or reducing the risk of developing or recurrence of a disease or symptom. “Curative” treatment includes reducing the severity of or suppressing the worsening of an existing disease, symptom, or condition. Thus, treatment includes ameliorating or preventing the worsening of existing disease symptoms, preventing additional symptoms from occurring, ameliorating or preventing the underlying metabolic causes of symptoms, inhibiting the disorder or disease, e.g., arresting the development of the disorder or disease, relieving the disorder or disease, causing regression of the disorder or disease, relieving a condition caused by the disease or disorder, or stopping the symptoms of the disease or disorder.

### Specific Embodiments

The invention is a method for treating a viral disease in a subject comprising administering to said subject a therapeutically effective dose of one or more of the compounds described in Formula (I):

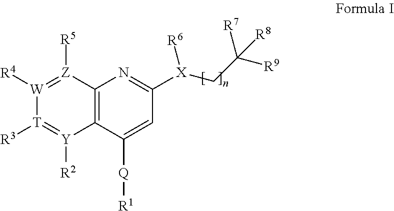

wherein each of R¹ and R⁶-R⁹ are independently —H, —CN, —COOH, —CONH₂, B(ORₐ)₂, an acid isostere, a halo, Cₙ alkyl, Cₙ alkenyl, Cₙ alkynyl, Cₙ aryl, Cₙ aminoalkyl, Cₙ haloalkyl, Cₙ heteroaryl, Cₙ cycloalkyl, or Cn heterocycloalkyl,

wherein Rₐ of the B(ORₐ)₂ is H or an alkyl,

wherein B of the B(ORₐ)₂ is boron,

wherein n of the Cₙ is 1-10

wherein each of R²-R⁵ are independently —H, —CN, —COOH, —COOMe, —CONH₂, B(ORₐ)₂, the acid isostere, the halo, —CONHOH, —NH—SO₂—C_{ι}-C₆-alkyl, —NHSO₂Ar, the Cₙ alkyl, the Cₙ alkyl, the Cₙ alkenyl, the Cₙ alkynyl, the Cₙ aryl, the Cₙ aminoalkyl, the Cₙ haloalkyl, the Cₙ heteroaryl, the Cₙ cycloalkyl, or the Cₙ heterocycloalkyl,

wherein the Ar of —NHSO₂Ar is selected from the group consisting of: phenyl, naphthyl, pyrrole, imidazole, thiophene, furan, thiazole, isothiazole, thiadiazole, oxazole, isoxazole, oxadiazole, pyridine, pyrazine, pyrimidine, pyridazine, pyrazole, triazole, tetrazole, chroman, isochroman, quinoline, quinoxaline, isoquinoline, phthalazine, cinnoline, quinazoline, indole, isoindole, indoline, isoindoline, benzothiophene, benzofuran, isobenzofuran, benzoxazole, 2,1,3-benzoxadiazole, benzothiazole, 2,1,3-benzothiazole, 2,1,3-benzoselenadiazole, benzimidazole, indazole, benzodioxane, indane, 1,2,3,4-tetrahydroquinoline, 3,4-dihydro-2H-1,4-benzoxazine, 1,5- naphthyridine, 1,8-naphthyridine, acridine, phenazine, and xanthene,

wherein each of the Cₙ alkyl, the Cₙ alkenyl, the Cₙ alkynyl, the Cₙ aryl, the Cₙ aminoalkyl, the Cₙ haloalkyl, the Cₙ heteroaryl, the Cₙ cycloalkyl, and the Cₙ heterocycloalkyl is unsubstituted or substituted with a quantity of substituents being between 1-5,

wherein each of the substituents is the same or different,

wherein each of the substituents is selected from the group consisting of H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, —C(O)alkyl, and —C_{q}—U—C_{q},

wherein each q of —C_{q}—U—C_{q} is independently 0 to 10,

wherein the U of —C_{q}—U—C_{q} is any one of aryl, heteroaryl, cycloalkyl, heterocycloalkyl, O, S, SO₂, or N(R₁)(R₁),

wherein each R₁ of N(R₁)(R₁) is independently hydrogen, the Cₙ alkyl, the Cₙ alkenyl, the Cₙ alkynyl, the Cₙ aryl, the Cₙ aminoalkyl, the Cₙ haloalkyl, the Cₙ heteroaryl, the Cₙ cycloalkyl, or the Cₙ heterocycloalkyl,

wherein each R₁ of N(R₁)(R₁) is unsubstituted or substituted with 1, 2, 3, 4 or 5 substituents which can be the same or different and are independently selected from the group consisting of: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, and —C(O)alkyl,

wherein Q is a bond or O,

wherein X is C, N, O, or S such that R⁶ is not present if X is O or S,

wherein “A” is a saturated or unsaturated ring depicted by

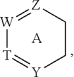

wherein Y, T, W, and Z are independently a bond, C, N, O, an alkyl having 1 to 4 carbon atoms, or an alkenyl having 1 to 4 carbon atoms, and

wherein n is 0, 1, 2, or 3.

In one embodiment, the invention comprises the compound of Formula I where at least one of R¹ and R², R² and R³, R³ and R⁴, R⁴ and R⁵, R⁶ and R⁷, R⁷ and R⁸, R⁸ and R⁹ are bonded forming a fused heteroaryl or fused heterocycloalkyl.

In a further embodiment, the invention may be a compound of Formula II or pharmaceutically acceptable salts or esters thereof:

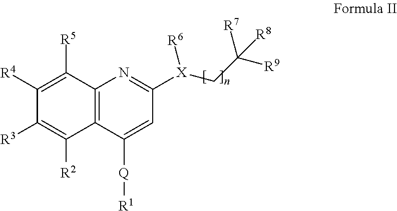

wherein R¹ and R⁶-R⁹ is independently —H, —CN, —COOH, —CONH₂, B(ORₐ)₂, an acid isostere, a halo, Cₙ alkyl, Cₙ alkenyl, Cₙ alkynyl, Cₙ aryl, Cₙ aminoalkyl, Cₙ haloalkyl, Cₙ heteroaryl, Cₙ cycloalkyl, or Cₙ heterocycloalkyl,

wherein Rₐ of the B(ORₐ)₂ is H or an alkyl,

wherein B of the B(ORₐ)₂ is boron,

wherein n of the Cₙ is 1 — 10,

wherein each of R² — Ware independently —H, —CN, —COOH, —COOMe, —CONH₂, B(ORₐ)₂, the acid isostere, the halo, —CONHOH, —NH—SO₂—C_{ι}-C₆-alkyl, —NHSO₂Ar, the Cₙ alkyl, the Cₙ alkyl, the Cₙ alkenyl, the Cₙ alkynyl, the Cₙ aryl, the Cₙ aminoalkyl, the Cₙ haloalkyl, the Cₙ heteroaryl, the Cₙ cycloalkyl, or the Cₙ heterocycloalkyl,

wherein the Ar of —NHSO₂Ar is selected from the group consisting of: phenyl, naphthyl, pyrrole, imidazole, thiophene, furan, thiazole, isothiazole, thiadiazole, oxazole, isoxazole, oxadiazole, pyridine, pyrazine, pyrimidine, pyridazine, pyrazole, triazole, tetrazole, chroman, isochroman, quinoline, quinoxaline, isoquinoline, phthalazine, cinnoline, quinazoline, indole, isoindole, indoline, isoindoline, benzothiophene, benzofuran, isobenzofuran, benzoxazole, 2,1,3-benzoxadiazole, benzothiazole, 2,1,3-benzothiazole, 2,1,3-benzoselenadiazole, benzimidazole, indazole, benzodioxane, indane, 1,2,3,4-tetrahydroquinoline, 3,4-dihydro-2H-1,4-benzoxazine, 1,5- naphthyridine, 1,8-naphthyridine, acridine, phenazine, and xanthene,

wherein each of the Cₙ alkyl, the Cₙ alkenyl, the Cₙ alkynyl, the Cₙ aryl, the Cₙ aminoalkyl, the Cₙ haloalkyl, the Cₙ heteroaryl, the Cₙ cycloalkyl, or the Cₙ heterocycloalkyl is unsubstituted or substituted with a quantity of substituents being between 1-5,

wherein each of the substituents is the same or different,

wherein each of the substituents is selected from the group consisting of: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, —C(O)alkyl, and —C_{q}—U—C_{q},

wherein each q of —C_{q}—U—C_{q} is independently 0 to 10,

wherein the U of —C_{q}—U—C_{q} is any one of aryl, heteroaryl, cycloalkyl, heterocycloalkyl, O, S, SO₂, or N(R₁)(R₁),

wherein each R₁ of N(R₁)(R₁) is independently hydrogen, the Cₙ alkyl, the Cₙ alkenyl, the Cₙ alkynyl, the Cₙ aryl, the Cₙ aminoalkyl, the Cₙ haloalkyl, the Cₙ heteroaryl, the Cₙ cycloalkyl, or the Cₙ heterocycloalkyl,

wherein each R₁ of N(R₁)(R₁) is unsubstituted or substituted with 1, 2, 3, 4 or 5 substituents which can be the same or different and are independently selected from the group consisting of: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, and —C(O)alkyl,

wherein Q is a bond or O,

wherein X is C, N, O, or S such that R⁶ is not present if X is O or S,

wherein X is C, N, O, or S, and

wherein n is 0, 1, 2, or 3.

In some embodiments, the invention includes compounds of Formula II, wherein a heterocycloalkyl group is formed by bonding two of R⁷, R⁸, or R⁹ to form:

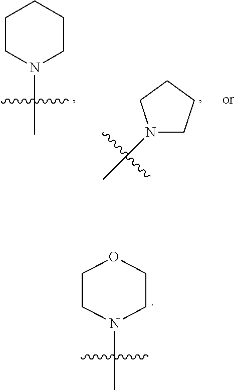 [SMILES: *N1CCCCC1 ; *N1CCCC1 ; *N1CCOCC1]

Additional embodiments of the invention include compounds of Formula III or pharmaceutically acceptable salts or esters thereof:

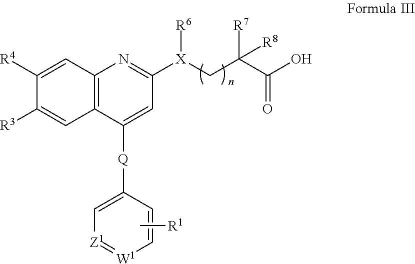

wherein R¹ is selected from the group consisting of: —CN, alkyl, —H, halo, ²H, amino, alkoxy, aminoalkyl, (amino)alkoxy, alkenyl, alkynyl, alkoxy, hydroxy, alkylhydroxy, aryloxy, alkyl(aryl), (alkoxyalkyl)amino, aryl, aryl(halo), heteroaryl, hydroxyl-alkyl, hydroxyl-aryl, (aryl)alkyl, C(O)OH, —S(O)₂-alkyl, —S(O)₂-aryl, —C(O)alkyl, and C(O)NH₂,

wherein each of R³ and R⁴ are independently —H, the halo, Cₙ alkyl, Cₙ alkyl, Cₙ alkenyl, Cₙ alkynyl, Cₙ aryl, Cₙ aminoalkyl, Cₙ haloalkyl, Cₙ heteroaryl, Cₙ cycloalkyl, Cₙ heterocycloalkyl, and —C_{q}—U—C_{q},

wherein n of Cn is 1 — 10,

wherein each q of —C_{q}—U—C_{q} is independently 0 to 10,

wherein the U of —C_{q}—U—C_{q} is any one of O, S, SO₂, or N(R₁)(R₁),

wherein each R₁ of N(R₁)(R₁) are independently hydrogen,

wherein each of the Cₙ alkyl, the Cₙ alkenyl, the Cₙ alkynyl, the Cₙ aryl, the Cn aminoalkyl, the Cn haloalkyl, the Cn heteroaryl, the Cₙ cycloalkyl, and the Cn heterocycloalkyl are unsubstituted or substituted with a quantity of substituents being between 1-5,

wherein each of the substituents are the same or different,

wherein each of the substituents is selected from the group consisting of: H, ²H, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, and —C(O)alkyl,

wherein R⁶ is independently H or alkyl,

wherein each of R⁷ and R⁸ are independently hydrogen, ²H, fluoro or alkyl,

wherein R⁶ and R⁷ are bonded with an adjoining R group to form a fused group,

wherein the fused group is selected from the group consisting of: a fused cycloalkyl, a fused heterocycloalkyl, a fused aryl, and a fused heteroaryl ring,

wherein the fused group comprises between 4 to 10 carbon atoms,

wherein n of Formula III is 0 or 1,

wherein Q is a bond or O,

wherein X is C, N, O, or S such that R⁶ is not present if X is O or S, and

wherein Z¹ or W¹ are independently C, N, O, or S.

Another embodiment of the invention includes compounds of Formula III wherein X is N.

An embodiment the invention includes compounds of Formula III wherein R⁶ is hydrogen and X is N.

A further embodiment the invention includes compounds of Formula III wherein R³ is alkyl and R¹ is cyano.

An additional embodiment the invention includes compounds of Formula III wherein X is N and n=0.

An embodiment the invention includes compounds of Formula III wherein X is N and n=1.

In one embodiment of the present invention there is a method of inhibiting the fatty acid binding protein FABP4 in a mammal, which comprises administering to a mammal an effective amount of a compound of Formula (I).

In one embodiment of the present invention the subject is a human.

In one embodiment of the present invention there is a compound according to Formula (I) for use in the prophylaxis or treatment of disorders acting on the fatty acid binding protein FABP4.

In one embodiment of the present invention the disorders are selected from common cold, SARS, and COVID-19.

In one embodiment of the present invention there is a pharmaceutical composition comprising a compound according to Formula (I) as the active ingredient.

In one embodiment of the present invention there is a pharmaceutical composition further comprising at least one additional active ingredient or a pharmaceutically acceptable carrier.

In one embodiment of the present invention there is a method for the prophylaxis or treatment of disorders acting on the fatty acid binding protein FABP4, which comprises administering to a subject in need of such treatment an effective amount of a compound according to Formula (I).

Any formula given herein is intended to represent compounds having structures depicted by the structural formula as well as certain variations or forms. For example, compounds of any formula given herein may have asymmetric or chiral centers and therefore exist in different stereoisomeric forms. All stereoisomers, including optical isomers, enantiomers, (+)-isomers, and diastereomers, of the compounds of the general formula, and mixtures thereof, are considered to fall within the scope of the formula. Furthermore, certain structures may exist as geometric isomers (i.e., cis and trans isomers), as tautomers, or as atropisomers. All such isomeric forms, and mixtures thereof, are contemplated herein as part of the present invention. Thus, any formula given herein is intended to represent a racemate, one or more enantiomeric forms, one or more diastereomeric forms, one or more tautomeric or atropisomeric forms, and mixtures thereof.

Diastereomeric mixtures may be separated into their individual diastereomers on the basis of their physical chemical differences by methods well known to those skilled in the art, such as, for example, by chromatography and/or fractional crystallization. Enantiomers may be separated by converting the enantiomeric mixture into a diastereomeric mixture by reaction with an appropriate optically active compound (e.g., chiral auxiliary such as a chiral alcohol or Mosher's acid chloride, or formation of a mixture of diastereomeric salts), separating the diastereomers and converting (e.g., hydrolyzing or de-salting) the individual diastereomers to the corresponding pure enantiomers. Enantiomers may also be separated by use of chiral HPLC column. The chiral centers of compounds of the present invention may be designated as “R” or “S” as defined by the IUPAC 1974 Recommendations.

The compounds of the invention can form pharmaceutically acceptable salts, which are also within the scope of this invention. A “pharmaceutically acceptable salt” refers to a salt of a free acid or base of a compound of Formula I, II, or III that is non-toxic, is physiologically tolerable, is compatible with the pharmaceutical composition in which it is formulated, and is otherwise suitable for formulation and/or administration to a subject. Reference to a compound herein is understood to include reference to a pharmaceutically acceptable salt of said compound unless otherwise indicated.

Compound salts include acidic salts formed with inorganic and/or organic acids, as well as basic salts formed with inorganic and/or organic bases. In addition, where a given compound contains both a basic moiety, such as, but not limited to, a pyridine or imidazole, and an acidic moiety, such as, but not limited to, a carboxylic acid, one of skill in the art will recognize that the compound may exist as a zwitterion (“inner salt”); such salts are included within the term “salt” as used herein. Salts of the compounds of the invention may be prepared, for example, by reacting a compound with an amount of a suitable acid or base, such as an equivalent amount, in a medium such as one in which the salt precipitates or in an aqueous medium followed by lyophilization.

Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate (“mesylate”), ethanesulfonate, benzenesulfonate, p-toluenesulfonate, and pamoate (i.e., 1,1′-methylene-bis(2-hydroxy-3-naphthoate)) salts. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counterions. Hence, a pharmaceutically acceptable salt can have one or more charged atoms and/or one or more counter ion.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates) and the like.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g., methyl, ethyl, and butyl chlorides, bromides and iodides), dialkyl sulfates (e.g., dimethyl, diethyl, and dibutyl sulfates), long chain halides (e.g., decyl, lauryl, and stearyl chlorides, bromides and iodides), aralkyl halides (e.g., benzyl and phenethyl bromides), and others.

Additionally, acids and bases which are generally considered suitable for the formation of pharmaceutically useful salts from pharmaceutical compounds are discussed, for example, by P. Stahl et al., Camille G. (eds.) Handbook of Pharmaceutical Salts: Properties, Selection and Use. (2002) Zurich: Wiley-VCH; S. Berge et al., J. Pharm. Sci. (1977) 66(1) 1-19; P. Gould, Int. J. Pharm. (1986) 33 201-217; Anderson et al., The Practice of Medicinal Chemistry (1996), Academic Press, New York; and in The Orange Book (Food & Drug Administration, MD, available from FDA). These disclosures are incorporated herein by reference thereto.

Additionally, any compound described herein is intended to refer also to any unsolvated form, or a hydrate, solvate, or polymorph of such a compound, and mixtures thereof, even if such forms are not listed explicitly. “Solvate” means a physical association of a compound of the invention with one or more solvent molecules. This physical association involves varying degrees of ionic and covalent bonding, including hydrogen bonding. In certain instances, the solvate will be capable of isolation, for example when one or more solvent molecules are incorporated in the crystal lattice of a crystalline solid. “Solvate” encompasses both solution-phase and isolatable solvates. Suitable solvates include those formed with pharmaceutically acceptable solvents such as water, ethanol, and the like. In some embodiments, the solvent is water and the solvates are hydrates.

One or more compounds of the invention may optionally be converted to a solvate. Methods for the preparation of solvates are generally known. Thus, for example, M. Caira et al., J. Pharm. Sci., 93(3), 601-611 (2004), describes the preparation of the solvates of the antifungal fluconazole in ethyl acetate as well as from water. Similar preparations of solvates, hemisolvate, hydrates, and the like are described by E. C. van Tonder et al., AAPS PharmSciTech., 5(1), article 12 (2004); and A. L. Bingham et al., Chem. Commun., 603-604 (2001). A typical, non-limiting process involves dissolving the inventive compound in a suitable amounts of the solvent (organic solvent or water or a mixture thereof) at a higher than ambient temperature, and cooling the solution at a rate sufficient to form crystals which are then isolated by standard methods. Analytical techniques such as, for example, infrared spectroscopy, show the presence of the solvent (or water) in the crystals as a solvate (or hydrate).

The invention also relates to pharmaceutically acceptable prodrugs of the compounds of Formula I, II, or III, and treatment methods employing such pharmaceutically acceptable prodrugs. The term “prodrug” means a precursor of a designated compound that, following administration to a subject, yields the compound in vivo via a chemical or physiological process such as solvolysis or enzymatic cleavage, or under physiological conditions (e.g., a prodrug on being brought to physiological pH is converted to the compound of Formula I). A “pharmaceutically acceptable prodrug” is a prodrug that is non-toxic, biologically tolerable, and otherwise suitable for formulation and/or administration to the subject. Illustrative procedures for the selection and preparation of suitable prodrug derivatives are described, for example, in “Design of Prodrugs”, ed. H. Bundgaard, Elsevier, 1985.

Examples of prodrugs include pharmaceutically acceptable esters of the compounds of the invention, which are also considered to be part of the invention. Pharmaceutically acceptable esters of the present compounds include the following groups: (1) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted with, for example, halogen, C₁₋₄alkyl, C₁₋₄alkoxy, or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di(C₆₋₂₄)acyl glycerol. Additional discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press.

For example, if a compound of Formula I, II, or III contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholine (C₂-C₃)alkyl, and the like.

Similarly, if a compound of Formula I, II, or III contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C4)alkanyl, arylacyl and α-aminoacyl, or α-aminoacyl- α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate), and the like.

If a compound of Formula I, II, or III incorporates an amine functional group, a prodrug can be formed by the replacement of a hydrogen atom in the amine group with a group such as, for example, R″-carbonyl, R″O-carbonyl, NR″R′-carbonyl where R″ and R′ are each independently (C₁-C₂₀)alkyl, (C₃-C7) cycloalkyl, benzyl, or R″-carbonyl is a natural α-aminoacyl or natural α-aminoacyl, —C(OH)C(O)OY¹ wherein Y¹ is H, (C₁-C₆)alkyl or benzyl, —C(OY²)Y³ wherein Y² is (C₁-C4) alkyl and Y³ is (C₁-C₆)alkyl, carboxy(C₁C₆)alkyl, amino(C₁C4)alkyl or mono-N— or di-N,N—(C₁-C₆)alkylaminoalkyl, —C(Y⁴)Y⁵ wherein Y⁴ is H or methyl and Y⁵ is mono-N— or di-N,N—(C₁-C₆)alkylamino morpholino, piperidin-1-yl or pyrrolidin-1-yl, and the like.

The present invention also relates to pharmaceutically active metabolites of compounds of Formula I, II, or III, and uses of such metabolites in the methods of the invention. A “pharmaceutically active metabolite” means a pharmacologically active product of metabolism in the body of a compound of Formula I, II, or III, or salt thereof. Prodrugs and active metabolites of a compound may be determined using routine techniques known or available in the art. See, e.g., Bertolini et al., J. Med. Chem. 1997, 40, 2011-2016; Shan et al., J. Pharm. Sci. 1997, 86 (7), 765-767; Bagshawe, Drug Dev. Res. 1995, 34, 220-230; Bodor, Adv. Drug Res. 1984, 13, 255-331; Bundgaard, Design of Prodrugs (Elsevier Press, 1985); and Larsen, Design and Application of Prodrugs, Drug Design and Development (Krogsgaard-Larsen et al., eds., Harwood Academic Publishers, 1991).

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, chlorine, and iodine, such as ²H, ³H, ¹¹C, ¹³C, ¹⁴C, ¹⁵N_{,} ¹⁸O, ¹⁷O, ³¹P, ³²P, ³⁵S, ¹⁸F, ³⁶Cl, and ¹²⁵I, respectively. Such isotopically labelled compounds are useful in metabolic studies (for example with ¹⁴C), reaction kinetic studies (with, for example ²H or ³H), detection or imaging techniques [such as positron emission tomography (PET) or single-photon emission computed tomography (SPECT)] including drug or substrate tissue distribution assays, or in radioactive treatment of patients. In particular, an ¹⁸F or ¹¹C labeled compound may be particularly suitable for PET or SPECT studies. Further, substitution with heavier isotopes such as deuterium (i.e., ²H) may afford certain therapeutic advantages resulting from greater metabolic stability, for example increased in vivo half-life or reduced dosage requirements. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

The use of the terms “salt,” “solvate,” “polymorph,” “prodrug,” and the like, with respect to the compounds described herein is intended to apply equally to the salt, solvate, polymorph, and prodrug forms of enantiomers, stereoisomers, rotamers, tautomers, atropisomers, and racemates of the inventive compounds.

The embodiments of the present invention can also include a compound from PCT/US21/14250 filed on Jan. 20, 2021, the entire contents of which are hereby incorporated by reference in their entirety. Such compounds may include: 3-{[6-butyl-4-(4-fluorophenyl)quinolin-2-yl](methyl)amino}-2-methylpropanoic acid, 3-{[4-(4-fluorophenyl)-6-hexylquinolin-2-yl](methyl)amino}-2-methylpropanoic acid, 2-{[4-(4-fluorophenyl)-6-pentylquinolin-2-yl](methyl)amino}acetic acid, 2-{[4-(4-fluorophenyl)-6-hexylquinolin-2-yl](methyl)amino}acetic acid, 2-{[6-hexyl-3-methyl-4-(morpholin-4-yl)quinolin-2-yl](methyl)amino}acetic acid, 2-{[4-(4-fluorophenyl)-6-hexyl-3-methylquinolin-2-yl](methyl)amino}acetic acid, 2{[4,6-bis(4-fluorophenyl)quinolin-2-yl](methyl)amino}acetic acid, 2-{[4-(4-fluorophenyl)-6-hexylquinolin-2-yl](2-methylpropyl)amino}acetic acid, 2-{[4-(4-fluorophenyl)-6-hexylquinolin-2-yl](propyl)amino}acetic acid, 2-{[4-(4-fluorophenyl)-6-hexylquinolin-2-yl]amino}acetic acid, 2-{ethyl[4-(4-fluorophenyl)-6-hexylquinolin-2-yl]amino}acetic acid, 2-{[6-hexyl-4-(pyridin-3-yloxy)quinolin-2-yl](methyl)amino}acetic acid, 2-{[6-hexyl-4-(pyridin-4-yloxy)quinolin-2-yl](methyl)amino}acetic acid, 2-{[4-(3-fluorophenoxy)-6-hexylquinolin-2-yl](methyl)amino}acetic acid, 2-{[4-(4-fluorophenoxy)-6-hexylquinolin-2-yl](methyl)amino}acetic acid, 2-{[4-(4-fluorophenyl)-6-octylquinolin-2-yl](2-methylpropyl)amino}acetic acid, 2-{[4-(4-fluorophenyl)-6-octylquinolin-2-yl](propyl)amino}acetic acid, 2-{ethyl[4-(4-fluorophenyl)-6-octylquinolin-2-yl]amino}acetic acid, 2-{methyl[6-octyl-4-(pyridin-3-yloxy)quinolin-2-yl]amino}acetic acid, 2-{methyl[6-octyl-4-(pyridin-4-yloxy)quinolin-2-yl]amino}acetic acid, 2-{[4-(3-fluorophenoxy)-6-octylquinolin-2-yl](methyl)amino}acetic acid, 2-{[4-(4-fluorophenoxy)-6-octylquinolin-2-yl](methyl)amino}acetic acid, 2-{[6-decyl-4-(4-fluorophenyl)quinolin-2-yl](methyl)amino}acetic acid, 2-{[4-(4-fluorophenyl)-6-heptylquinolin-2-yl](methyl)amino}acetic acid, 2-{[4-(4-fluorophenyl)-6-octylquinolin-2-yl](methyl)amino}acetic acid, 2-[(6-hexylquinolin-2-yl)(methyl)amino]acetic acid, 2-{2-[(carboxymethyl)(methyl)amino]-6-hexylquinolin-4-yl}benzoic acid, 2-{[4-(4,4-difluoropiperidin-1-yl)-6-hexylquinolin-2-yl](methyl)amino}acetic acid, 2-{[4-(3,-difluoropyrrolidin-1-yl)-6-hexylquinolin-2-yl](methyl)amino}acetic acid, 2-{[6-hexyl-4-(morpholin-4-yl)quinolin-2-yl](methyl)amino}acetic acid, 2-{[6-butyl-4-(2-methyl-pyridin-4-yl)quinolin-2-yl](methyl)amino}acetic acid, 2-{[4-(3,5-dimethyl-1,2-oxazol-4-yl)-6-hexylquinolin-2-yl](methyl)amino}acetic acid, 2-{[4-(3-cyanophenyl)-6-hexylquinolin-2-yl](methyl)amino}acetic acid, 3-{[4-(3-cyanophenyl)-6-hexylquinolin-2-yl](methyl)amino}butanoic acid, 3-[(6-hexyl-4-phenylquinolin-2-yl)(methyl)amino]-2-methylpropanoic acid, 2-[methyl(6-pentanamido-4-phenylquinolin-2-yl)amino]acetic acid, 2-{methyl[6-(pentyloxy)-4-phenylquinolin-2-yl]amino}acetic acid, 2-[(7-bromo-4-phenylquinolin-2-yl)(methyl)amino] acetic acid, 2-[(7-hexyl-4-phenylquinolin-2-yl)(methyl)amino]acetic acid, 2-[methyl(6-octyl-4-phenylquinolin-2-yl)amino]acetic acid, 3-{[6-hexyl-4-(pyridin-3-yl)quinolin-2-yl](methyl)amino}-2-methylpropanoic acid, 2-{[6-hexyl-4-(pyridin-3-yl)quinolin-2-yl](methyl)amino}acetic acid, 2-{[4-(3-cyanophenyl)-6-hexylquinolin-2-yl]oxy}acetic acid, 3-{[4-(3-cyanophenyl)-6-hexylquinolin-2-yl](methyl)amino}-2-methylpropanoic acid, 2-{[443-cyanophenyl)-6-hexylquinolin-2-yl](methyl)amino}acetic acid, 1-[6-hexyl-4-(pyridin-3-yl)quinolin-2-yl]piperidine-3-carboxylic acid, 1-(6-hexyl-4-phenylquinolin-2-yl)piperidine-3-carboxylic acid, 2-{[6-butyl-4-(4-hydroxyphenyl)quinolin-2-yl](methyl)amino}acetic acid, 2-{[6-butyl-4-(3-hydroxyphenyl)quinolin-2-yl](methyl)amino} acetic acid, 2-{[6-butyl-4-(2-hydroxyphenyl)quinolin-2-yl](methyl)amino}acetic acid, 2-{[6-butyl-4-(4-fluorophenyl)quinolin-2-yl](methyl)amino}acetic acid, 2-{[6-butyl-4-(3-fluorophenyl)quinolin-2-yl](methyl)amino}acetic acid, 2-{[6-butyl-4-(2-fluorophenyl)quinolin-2-yl](methyl)amino}acetic acid, 2-{[6-butyl-4-(4-methylphenyl)quinolin-2-yl](methyl)amino}acetic acid, 2-{[6-butyl-4-(3-methylphenyl)quinolin-2-yl](methyl)amino}acetic acid, 2-{[6-butyl-4-(2-methylphenyl)quinolin-2-yl](methyl)amino}acetic acid, 2{[6-butyl-4-(4-cyanophenyl)quinolin-2-yl](methyl)amino}acetic acid, 2-{[6-butyl-4-(4-carbamoylphenyl)quinolin-2-yl](methyl)amino}acetic acid, 2-{[6-butyl-4-(pyridin-4-yl)quinolin-2-yl](methyl)amino}acetic acid, 6-butyl-2-(carboxymethoxy)-4-phenylquinoline-3-carboxylic acid, 2-{[6-butyl-4-(pyridin-3-yl)quinolin-2-yl](methyl)amino}acetic acid, 1-[6-butyl-4-(3-cyanophenyl)quinolin-2-yl]piperidine-3-carboxylic acid, 4-(6-butyl-4-phenylquinolin-2-yl)morpholine-2-carboxylic acid, 1-(6-butyl-4-phenylquinolin-2-yl)piperidine-3-carboxylic acid, 3-{[6-butyl-4-(3-cyanophenyl)quinolin-2-yl](methyl)amino}-2-methylpropanoic acid, 3-{[6-butyl-4-(pyridin-3-yl)quinolin-2-yl](methyl)amino}-2-methylpropanoic acid, 3-[(6-butyl-4-phenylquinolin-2-yl)(methyl)amino]-2-methylpropanoic acid, 3-[(6-butyl-4-phenylquinolin-2-yl)(methyl)amino]butanoic acid, 3-[(6-butyl-4-phenylquinolin-2-yl)(methyl)amino]propanoic acid, N-(6-butyl-4-(3-cyanophenyl)quinolin-2-yl)-N-methylvaline, 2-{[4-(3-cyanophenyl)-6-pentylquinolin-2-yl]oxy}acetic acid, 2-{[4-(3-carbamoylphenyl)-6-pentylquinolin-2-yl]oxy}acetic acid, 2-{[4-(3-cyanophenyl)-6-propylquinolin-2-yl]oxy}acetic acid, 2-{[4-(3-carbamoylphenyl)-6-propylquinolin-2-yl]oxy}acetic acid, 2-{[4-(3-carbamoylphenyl)-6-ethylquinolin-2-yl]oxy}acetic acid, 2-{[6-bromo-4-(3-cyanophenyl)quinolin-2-yl]oxy}acetic acid, 2-{[6-bromo-4-(3-carbamoylphenyl)quinolin-2-yl]oxy}acetic acid, 2-{[6-butyl-4-(3-cyanophenyl)quinolin-2-yl]oxy}acetic acid, 2-{[6-butyl-4-(3-carbamoylphenyl)quinolin-2-yl]oxy}acetic acid, 2-{[4-(3-cyanophenyl)-6-pentylquinolin-2-yl](methyl)amino}acetic acid, 2-{[4-(3-carbamoylphenyl)-6-pentylquinolin-2-yl](methyl)amino}acetic acid, 2-{[4-(3-cyanophenyl)-6-propylquinolin-2-yl](methyl)amino}acetic acid, 2-{[4-(3-carbamoylphenyl)-6-propylquinolin-2-yl](methyl)amino}acetic acid, 2-{[4-(3-cyanophenyl)-6-ethylquinolin-2-yl](methyl)amino}acetic acid, 2-{[4-(3-carbamoylphenyl)-6-ethylquinolin-2-yl](methyl)amino}acetic acid, 2-{[6-bromo-4-(3-cyanophenyl)quinolin-2-yl](methyl)amino}acetic acid, 2-{[6-bromo-4-(3-carbamoylphenyl)quinolin-2-yl](methyl)amino}acetic acid, 2-{[6-butyl-4-(3-cyanophenyl)quinolin-2-yl](methyl)amino}acetic acid, 2-{[6-butyl-4-(3-carbamoylphenyl)quinolin-2-yl](methyl)amino}acetic acid, 2-{2-[(6-chloro-4-phenylquinolin-2-yl)(methyl)amino]acetamido}acetic acid, 2-[methyl(6-pentyl-4-phenylquinolin-2-yl)amino]acetic acid, 2-[methyl(4-phenyl-6-propylquinolin-2-yl)amino]acetic acid, 2-[(6-ethyl-4-phenylquinolin-2-yl)(methyl)amino]acetic acid, 1-(6-hexyl-4-phenylquinolin-2-yl)pyrrolidine-2-carboxylic acid, 6-hexyl-4-phenyl-2-(piperidin-1-yl)quinoline, 2-[(6-hexyl-4-phenylquinolin-2-yl)(methyl)amino]acetic acid, 1-(6-butyl-4-phenylquinolin-2-yl)pyrrolidine-2-carboxylic acid, 6-butyl-4-phenyl-2-(piperidin-1-yl)quinoline, 2-[(6-bromo-4-phenylquinolin-2-yl)oxy]acetic acid, 2-[(6-bromo-4-phenylquinolin-2-yl)(methyl)amino]acetic acid, 2-[(6-pentyl-4-phenylquinolin-2-yl)oxy]acetic acid, 2-[(4-phenyl-6-propylquinolin-2-yl)oxy]acetic acid, 2-[(6-ethyl-4-phenylquinolin-2-yl)oxy]acetic acid, 2-[(6-chloro-4-phenylquinolin-2-yl)oxy]acetic acid, 2-[(6-butyl-4-phenylquinolin-2-yl)oxy]acetic acid, 2-[(6-butyl-4-phenylquinolin-2-yl)(methyl)amino]acetic acid, 1-(6-chloro-4-phenylquinolin-2-yl)pyrrolidine-2-carboxylic acid, 2-[(6-chloro-4-phenylquinolin-2-yl)(methyl)amino]propanoic acid, 2-[(6-chloro-4-phenylquinolin-2-yl)(methyl)amino]acetic acid, 6-chloro-4-phenyl-2-(piperidin-1-yl)quinoline, 3-{[6-butyl-4-(4-fluorophenyl)quinolin-2-yl](methyl)amino}-2-methylpropanoic acid, 2-[methyl(6-octyl-4-phenylquinolin-2-yl)amino]acetic acid, 2-{[6-butyl-4-(4-fluorophenyl)quinolin-2-yl](methyl)amino}acetic acid, 2-{methyl[6-octyl-4-(pyridin-3-yloxy)quinolin-2-yl]amino}acetic acid, 2-{[6-butyl-4-(3-fluorophenyl)quinolin-2-yl](methyl)amino}acetic acid, 2-[(6-hexyl-4-phenylquinolin-2-yl)(methyl)amino]acetic acid, 2-{[4-(4-fluorophenoxy)-6-octylquinolin-2-yl](methyl)amino} acetic acid, 2-{[6-butyl-4-(3-methylphenyl)quinolin-2-yl](methyl)amino}acetic acid, 2-[(6-butyl-4-phenylquinolin-2-yl)(methyl)amino]acetic acid, 2-{[6-decyl-4-(4-fluorophenyl)quinolin-2-yl](methyl)amino}acetic acid, 2-[methyl(4-phenyl-6-propylquinolin-2-yl)amino]acetic acid, 2-{[6-butyl-4-(4-methylphenyl)quinolin-2-yl](methyl)amino}acetic acid, 3-[(6-butyl-4-phenylquinolin-2-yl)(methyl)amino]-2-methylpropanoic acid, 2-{[4-(4-fluorophenyl)-6-pentylquinolin-2-yl](methyl)amino}acetic acid, 2{[6-butyl-4-(3-cyanophenyl)quinolin-2-yl](methyl)amino}acetic acid, 2-{[6-butyl-4-(2-fluorophenyl)quinolin-2-yl](methyl)amino}acetic acid, 2-{[6-hexyl-4-(pyridin-3-yloxy)quinolin-2-yl](methyl)amino}acetic acid, 2-{[4-(3-fluorophenoxy)-6-octylquinolin-2-yl](methyl)amino}acetic acid, 2-[methyl(6-pentyl-4-phenylquinolin-2-yl)amino]acetic acid, 3-[(6-hexyl-4-phenylquinolin-2-yl)(methyl)amino]-2-methylpropanoic acid, 2-{[4-(3-cyanophenyl)-6-propylquinolin-2-yl](methyl)amino}acetic acid, 2-{[6-hexyl-4-(morpholin-4-yl)quinolin-2-yl](methyl)amino}acetic acid, 3-{[4-(4-fluorophenyl)-6-hexylquinolin-2-yl](methyl)amino}-2-methylpropanoic acid, 2{[4-(4-fluorophenyl)-6-hexylquinolin-2-yl](methyl)amino}acetic acid, 3-{[4-(3-cyanophenyl)-6-hexylquinolin-2-yl](methyl)amino}-2-methylpropanoic acid, 2-[methyl({6-[2-(4-methylphenyl)ethyl]-4-phenylquinolin-2-yl})amino]acetic acid, 2-[methyl({6-[2-(3-methylphenyl)ethyl]-4-phenylquinolin-2-yl})amino]acetic acid, 6-hexyl-N-methyl-4-phenyl-N-[(2H-1,2,3,4-tetrazol-5-yl)methyl]quinolin-2-amine, 2-{methyl[4-phenyl-6-(2-phenylethyl)quinolin-2-yl]amino}acetic acid, 2-({6-[2-(3-chlorophenyl)ethyl]-4-phenylquinolin-2-yl}(methyl)amino)acetic acid, 5-{[4-phenyl-6-(2-phenylethyl)quinolin-2-yl]methyl }-1,3-thiazolidine-2,4-dione, 2-({6-[2-(4-chlorophenyl)ethyl]-4-phenylquinolin-2-yl}(methyl)amino)acetic acid, 2-[methyl({4-phenyl-6-[2-(pyridin-3-yl)ethyl]quinolin-2-yl})amino]acetic acid, 2-[methyl({4-phenyl-6-[2-(quinolin-6-y1)ethyl]quinolin-2-yl})amino]acetic acid, 2-[(6-heptyl-4-phenylquinolin-2-yl)(methyl)amino]acetic acid, 2-[methyl({6-[2-(2-methylphenyl)ethyl]-4-phenylquinolin-2-yl})amino]acetic acid, 2-[methyl({4-phenyl-6-[2-(pyridin-2-yl)ethyl]quinolin-2-yl})amino]acetic acid, cis-2-(6-hexyl-4-phenylquinolin-2-yl)cyclopropane-1-carboxylic acid, 1-(6-hexyl-4-phenylquinolin-2-yl)-3-methylpyrrolidine-3-carboxylic acid, 2-[methyl({4-phenyl-6-[2-(pyrimidin-2-yl)ethyl]quinolin-2-yl})amino]acetic acid, 2-[(6-hexyl-4-phenyl-5,6,7,8-tetrahydroquinolin-2-yl)(methyl)amino]acetic acid, 2-[methyl({4-phenyl-6-[2-(quinoxalin-6-y1)ethyl]quinolin-2-yl})amino]acetic acid, 5-[(6-hexyl-4-phenylquinolin-2-yl)methyl]-1,3-thiazolidine-2,4-dione, 2-({6-[(1E)-hex-1-en-1-yl]-4-phenylquinolin-2-yl}(methyl)amino)acetic acid, 2-{methyl[4-phenyl-6-(3-phenylpropyl)quinolin-2-yl]amino}acetic acid, 2-[methyl({4-phenyl-6-[2-(1,2,3,4-tetrahydroquinolin-6-yl)ethyl]quinolin-2-yl})amino]acetic acid, 2-({6-[2-(3-methoxyphenyl)ethyl]-4-phenylquinolin-2-yl}(methyl)amino)acetic acid, 2-({6-[2-(1,3-benzothiazol-2-yl)ethyl]-4-phenylquinolin-2-yl}(methyl)amino)acetic acid, 2-{[6-hexyl-4-(pyridin-4-yl)quinolin-2-yl]oxy}propanoic acid, or 3-(6-hexyl-4-phenylquinolin-2-yl)butanoic acid.

It is an object of the present invention to utilize the compounds described herein in the treatment of viral disorders by acting on the fatty acid binding protein (FABP4).

Yet another object of the present invention is a pharmaceutical composition comprising a compounds herein as active ingredient, in combination with a pharmaceutically acceptable diluent or carrier for use in the treatment of viral disorders by acting on FABP4. Here, the pharmaceutical composition can further comprise an additional therapeutically active agent.

Yet another object of the present invention is a method for the treatment of viral disorders by acting on the FABP4, which comprises administering to a subject in need of such treatment (preferably, a human) an effective amount of the compounds herein, including, optionally, the co-administration with other therapeutic agents, either as a single (or multiple) dosing, and either simultaneously or sequentially.

Yet another embodiment is a method for administering a compound of the instant invention to a subject (e.g., a human) in need thereof by administering to the subject the pharmaceutical formulation of the present invention.

Yet another embodiment is a method of preparing a pharmaceutical formulation of the present invention by mixing at least one pharmaceutically acceptable compound of the present invention, and, optionally, one or more pharmaceutically acceptable additives or excipients.

For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g., magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18th Edition, (1990), Mack Publishing Co., Easton, Pa.

The compositions and formulations of the invention can be administered as sterile compositions and sterile formulations. Sterile pharmaceutical formulations are compounded or manufactured according to pharmaceutical-grade sterilization standards (e.g., United States Pharmacopeia Chapters 797, 1072, and 1211; California Business & Professions Code 4127.7; 16 California Code of Regulations 1751, 21 Code of Federal Regulations 21, or ex-U.S. counterparts to such regulations) known to those of skill in the art.

Liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g. nitrogen.

Methods of delivering drugs by pulmonary administration have been described. For example, each of U.S. Pat. Nos. 6,550,472, 6,546,927, 6,543,443, 6,540,154, 6,540,153, 6,467,476, 6,427,682, 6,503,480, 6,447,753, 6,387,390, 5,985,320, 5,985,309, 5,855,913, 6,431,167, 6,408,854, 6,349,719, 6,167,880, 6,098,620, 5,971,951, 5,957,124, 5,906,202, 5,819,726, 5,755,218, 5,522,385, 6,546,929, 6,543,448, 6,509,006, 6,423,344, 6,303,582, and 6,138,668 teaches methods and devices useful in the pulmonary administration of drugs and/or nasal instillation. Bioadhesives have been described for facilitating transport of medicaments across endothelial mucosa. For example, U.S. Pat. No. 6,228,383 teaches use of bioadhesive fatty acid esters for facilitating transport of drug substances across mucosa in the lung, nose and other tissues. Penetration enhancers have been described in, for example, U.S. patent application Ser. No. 09/315,298, filed on May 20, 1999. Penetration enhancers facilitate the penetration of mucosa, including pulmonary and nasal mucosa. The present invention provides, inter alia, compositions formulated for pulmonary or nasal administration of antiviral compounds, especially compounds capable of attenuating, mitigating or preventing viral infections, and especially coronavirus. In some embodiments of the invention, viral infections are treated by administering an antiviral compound of the present invention to a patient in need thereof. In some embodiments of the invention, viral infection is treated by administering an antiviral small molecule to a patient in need thereof. In some embodiments according to the present invention, an antiviral agent is administered by pulmonary or intranasal means to a patient in need thereof. In certain embodiments, the antiviral agent is a compound of the invention. In other embodiments of the invention, the antiviral agent is a mixture of antiviral compounds. In certain embodiments, the invention comprises a molecule of the invention in combination therapy, for example with one or more additional antiviral agents. In some embodiments of the present invention, the inventive composition comprises, in addition to one or more antiviral agents, a therapeutically acceptable agent for intrapulmonary or intranasal administration.

Also included are solid form preparations that are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

The compounds of this invention may also be delivered subcutaneously.

The compound can be administered orally or intravenously.

The pharmaceutical preparation can be in a unit dosage form. In such form, the preparation is subdivided into suitably sized unit doses containing appropriate quantities of the active component, e.g., an effective amount to achieve the desired purpose.

The quantity of active compound in a unit dose of preparation may be varied or adjusted from about 1 mg to about 1000 mg, for example from about 1 mg to about 500 mg, in particular from about 1 mg to about 250 mg, or from about 1 mg to about 25 mg, according to the particular application.

The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

The amount and frequency of administration of the compounds of the invention and/or the pharmaceutically acceptable salts thereof will be regulated according to the judgment of the attending clinician considering such factors as age, condition and size of the patient as well as severity of the symptoms being treated. A typical recommended daily dosage regimen for oral administration can range from about 1 mg/day to about 500 mg/day, preferably 1 mg/day to 200 mg/day, in two to four divided doses.

Treatment or Prevention of Viral Infection

The compounds of the invention are useful in human and veterinary medicine for treating or preventing a viral infection in a patient. In one embodiment, the compounds of the invention are inhibitors of viral replication. In another embodiment, the compounds of the invention can be inhibitors of influenza, rhinovirus or coronavirus replication. Accordingly, the compounds of the invention are useful for treating viral infections, such as coronavirus. In accordance with the invention, the compounds of the invention can be administered to a patient in need of treatment or prevention of a viral infection.

Accordingly, in one embodiment, the invention provides methods for treating a viral infection in a patient comprising administering to the patient an effective amount of at least one compounds of the invention or a pharmaceutically acceptable salt thereof.

The compounds of the invention are useful in the inhibition of viruses, the treatment of viral infection and/or reduction of the likelihood or severity of symptoms of viral infection and the inhibition of viral replication and/or viral production in a cell-based system. For example, the compounds of the invention are useful in treating infection by viruses after suspected past exposure to viruses by such means as airborne transmission, blood transfusion, exchange of body fluids, etc.

In one embodiment, the viral infection is acute respiratory viral infection. In another embodiment, the viral infection is chronic viral infection.

Accordingly, in one embodiment, the invention provides methods for treating viral infection in a patient, the methods comprising administering to the patient an effective amount of at least one the invention or a pharmaceutically acceptable salt thereof. In a specific embodiment, the amount administered is effective to treat or prevent a viral infection in a patient. In another specific embodiment, the amount administered is effective to inhibit viral replication and/or viral production in the patient.

In particular embodiments, the viral infection is caused by influenza, coronavirus, rhinovirus, respiratory syncytial viruses (RSVs), parainfluenza, adenoviruses, Human metapneumovirus (HMPV) or Human Bocavirus (HBoV).

Combination Therapy

In one embodiment, one or more compounds of the present invention are administered with one or more additional therapeutic agents selected from: an interferon, an immunomodulator, a viral replication inhibitor, an antisense agent, a therapeutic vaccine, a viral polymerase inhibitor, a nucleoside inhibitor, a viral protease inhibitor, a viral helicase inhibitor, a viral polymerase inhibitor a virion production inhibitor, a viral entry inhibitor, a viral assembly inhibitor, an antibody therapy (monoclonal or polyclonal), and any agent useful for treating an RNA-dependent polymerase-related disorder.

In another embodiment, the present methods for treating or preventing viral infection can further comprise the administration of one or more additional therapeutic agents.

In one embodiment, the additional therapeutic agent is an antiviral agent.

In another embodiment, the additional therapeutic agent is an immunomodulatory agent, such as an immunosuppressive agent.

Accordingly, in one embodiment, the present invention provides methods for treating a viral infection in a patient, the method comprising administering to the patient: a compound of the invention or a pharmaceutically acceptable salt thereof, and (ii) at least one additional therapeutic agent that is other than a compound of the invention, wherein the amounts administered are together effective to treat or prevent a viral infection.

When administering a combination therapy of the invention to a patient, therapeutic agents in the combination, or a pharmaceutical composition or compositions comprising therapeutic agents, may be administered in any order such as, for example, sequentially, concurrently, together, simultaneously and the like. The amounts of the various actives in such combination therapy may be different amounts (different dosage amounts) or same amounts (same dosage amounts). Thus, for non-limiting illustration purposes, a compound of the invention and an additional therapeutic agent may be present in fixed amounts (dosage amounts) in a single dosage unit (e.g., a capsule, a tablet and the like).

In one embodiment, the at least one a compound of the invention is administered during a time when the additional therapeutic agent(s) exert their prophylactic or therapeutic effect, or vice versa.

In another embodiment, the at least one a compound of the invention and the additional therapeutic agent(s) are administered in doses commonly employed when such agents are used as monotherapy for treating a viral infection.

In another embodiment, the at least one a compound of the invention and the additional therapeutic agent(s) are administered in doses lower than the doses commonly employed when such agents are used as monotherapy for treating a viral infection.

In still another embodiment, the at least one a compound of the invention and the additional therapeutic agent(s) act synergistically and are administered in doses lower than the doses commonly employed when such agents are used as monotherapy for treating a viral infection.

In one embodiment, the at least one a compound of the invention and the additional therapeutic agent(s) are present in the same composition. In one embodiment, this composition is suitable for oral administration. In another embodiment, this composition is suitable for intravenous administration. In another embodiment, this composition is suitable for subcutaneous administration. In still another embodiment, this composition is suitable for parenteral administration.

Viral infections and virus-related disorders that can be treated or prevented using the combination therapy methods of the present invention include, but are not limited to, those listed above.

The at least one a compound of the invention and the additional therapeutic agent(s) can act additively or synergistically. A synergistic combination may allow the use of lower dosages of one or more agents and/or less frequent administration of one or more agents of a combination therapy. A lower dosage or less frequent administration of one or more agents may lower toxicity of therapy without reducing the efficacy of therapy.

In one embodiment, the administration of at least one 5 a compound of the invention and the additional therapeutic agent(s) may inhibit the resistance of a viral infection to these agents.

Non-limiting examples of additional therapeutic agents useful in the present compositions and methods include an interferon, an immunomodulator, a viral replication inhibitor, an antisense agent, a therapeutic vaccine, a viral polymerase inhibitor, a nucleoside inhibitor, a viral protease inhibitor, a viral helicase inhibitor, a virion production inhibitor, a viral entry inhibitor, a viral assembly inhibitor, an antibody therapy (monoclonal or polyclonal), and any agent useful for treating an RNA-dependent polymerase-related disorder.

In one embodiment, the additional therapeutic agent is a viral protease inhibitor.

In another embodiment, the additional therapeutic agent is a viral replication inhibitor.

In another embodiment, the compounds of the invention are in substantially purified form.

Other embodiments of the present invention include the following:

(a) A pharmaceutical composition comprising an effective amount of a compound of the invention or a pharmaceutically acceptable salt thereof, and a pharmaceutically acceptable carrier.

(b) The pharmaceutical composition of (a), further comprising a second therapeutic agent selected from the group consisting of antiviral agents, immunomodulators, and anti-infective agents.

(c) The pharmaceutical composition of (b), wherein the antiviral agent is an antiviral selected from the group consisting of protease inhibitors, polymerase inhibitors and other viral inhibitors.

(d) A pharmaceutical combination that is (i) a compound of the invention and (ii) a second therapeutic agent selected from the group consisting of antiviral agents, immunomodulators, and anti-infective agents; wherein the compound of the invention and the second therapeutic agent are each employed in an amount that renders the combination effective for inhibiting viral replication or disease, or for treating viral infection and/or reducing the likelihood or severity of symptoms of viral infection.

(e) The combination of (d), wherein the antiviral agent is an antiviral selected from the group consisting of protease inhibitors, polymerase inhibitors and other viral inhibitors.

(f) A method of inhibiting viral replication in a subject in need thereof which comprises administering to the subject an effective amount of a compound of the invention.

(g) A method of treating viral infection and/or reducing the likelihood or severity of symptoms of viral infection in a subject in need thereof which comprises administering to the subject an effective amount of a compound of the invention).

(h) The method of (g), wherein the compound of the invention is administered in combination with an effective amount of at least one second therapeutic agent selected from the group consisting of antiviral agents, immunomodulators, and anti-infective agents.

(i) The method of (h), wherein the antiviral agent is an antiviral selected from the group consisting of protease inhibitors, polymerase inhibitors and other viral inhibitors.

(j) A method of inhibiting viral replication in a subject in need thereof which comprises administering to the subject the pharmaceutical composition of (a), (b) or (c) or the combination of (d) or (e).

(k) A method of treating viral infection and/or reducing the likelihood or severity of symptoms of viral infection in a subject in need thereof which comprises administering to the subject the pharmaceutical composition of (a), (b) or (c) or the combination of (d) or (e).

The present invention also includes a compound of the present invention for use (i) in, (ii) as a medicament for, or (iii) in the preparation of a medicament for: (a) medicine, (b) inhibiting viral replication or (c) treating viral infection and/or reducing the likelihood or severity of symptoms of viral infection. In these uses, the compounds of the present invention can optionally be employed in combination with one or more second therapeutic agents selected from antiviral agents, anti-infective agents, and immunomodulators.

Additional embodiments of the invention include the pharmaceutical compositions, combinations and methods set forth in (a)-(k) above and the uses set forth in the preceding paragraph, wherein the compound of the present invention employed therein is a compound of one of the embodiments, aspects, classes, sub-classes, or features of the compounds described above. In all of these embodiments, the compound may optionally be used in the form of a pharmaceutically acceptable salt or hydrate as appropriate. It is understood that references to compounds would include the compound in its present form as well as in different forms, such as polymorphs, solvates and hydrates, as applicable.

It is further to be understood that the embodiments of compositions and methods provided as (a) through (k) above are understood to include all embodiments of the compounds, including such embodiments as result from combinations of embodiments.

### Detailed Description of Experiments

Synthetic method A:

Representative example: 2-{[4-(3-cyanophenyl)-6-hexylquinolin-2-yl](methyl)amino}acetic acid

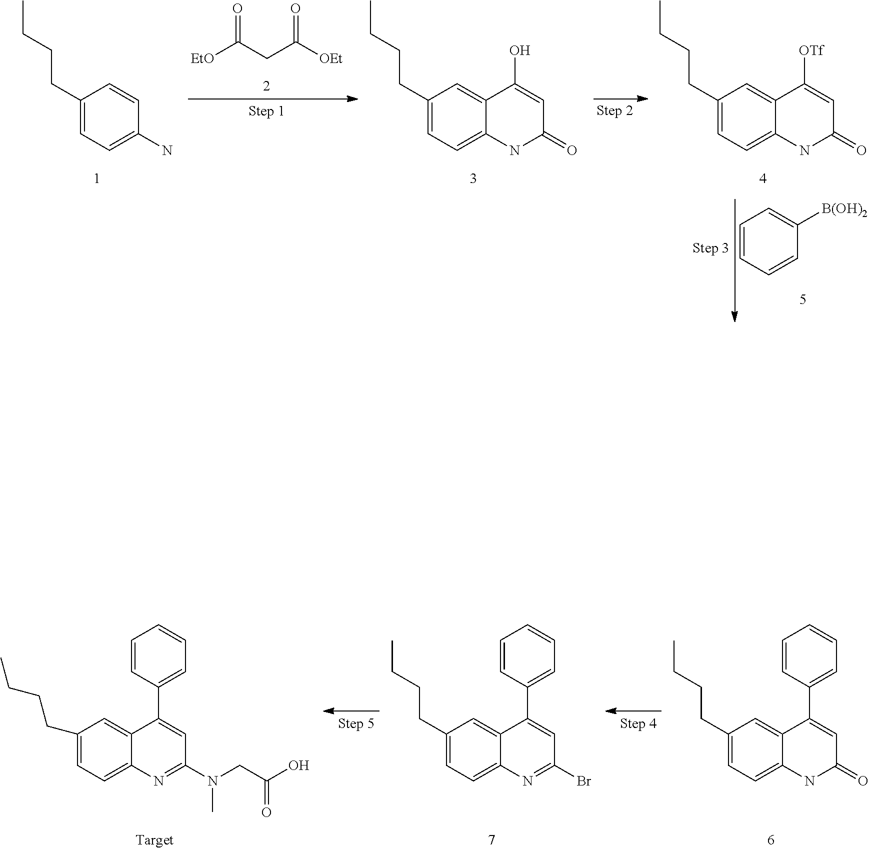

4-hexyl aniline (5 g, 28.24 mmol) in CH₂Cl₂ (50 mL) was cooled to 0° C. and then diethyl malonate 2 was added at stirred for 2 h at room temperature. Reaction mixture was quenched with ice and neutralized with saturated NaHCO₃ solution. Resulting solution was then extracted with CH₂Cl₂ (×2) then dried over Na₂SO₄ and concentrated. 5 g of desired ester amide product was obtained by purification by flash column chromatography which was taken to next step.

5 g of ester amide in PolyPhosphoricAcid (50 mL) was heated to 150° C. and stirred. After 4 h, the reaction was poured into ice and stirred. Pale brown solid was formed after 30 minutes which was filtered and dried to yield 3.5 g of the desired bicyclic product, as determined by LCMS and HNMR.

Bicyclic beta-keto amide (3.5 g, 14.285 mmol) in 15 mL of DMF was cooled to 0° C. and triethylamine (2.48 mL, 17.142 mmol) was added and stirred for 5 min. Comin's reagent (3.78 g, 17.142 mmol) was added and the reaction mixture was stirred for 2 h at room temperature. Reaction mixture was then poured into ice and pale yellow solid was formed which was filtered and dried to yield 3.0 g of the desired product, as determined by LCMS and HNMR.

Bicyclic triflate (0.3 g, 0.797 mmol), 3-cyano-phenyl boronic acid (0.177 g, 1.196 mmol) and Na₂CO₃ (0.168 g, 1.594 mmol) was added in DMF+H₂O (10 mL:2 mL) solution. Argon gas was bubbled and Pd(dppf)Cl₂ (0.058 g, 0.0797 mmol) was added at which point the reaction was moved to pre-heated 100° C. oil bath and stirred for 1h. Reaction mixture was diluted with water, resulting in formation of brown precipitation which was then filtered and dried to yield the desired product, as determined by LCMS and HNMR.

Tricyclic amide (0.2 g, 0.606 mmol) in toluene was added POBr₃ (0.52 g, 1.818 mmol) and stirred for 3 h at 140° C. The reaction mixture was poured into ice and the aqueous layer was extract with EtOAc (×2). The organic layer was then dried over Na₂SO₄ and concentrated. Purification by flash column chromatography gave the desired bromide product as determined by LCMS and HNMR.

Cyano bromide (0.18 g, 0.459 mmol) in DMSO (5 mL) was added N-Methyl glycine (0.32 g, 2.295 mmol) and K₂CO₃ (0.19 g, 1.377 mmol). Reaction mixture was then stirred at 100oC for 16 h before quenched with H₂O. The mixture was acidified with 1N HCl to adjust the pH˜6 before being extracted with EtOAc (×2). Organic layer was dried over Na₂SO₄ and concentrated before purification by reverse phase prep-HPLC to yield 2-{[4-(3-cyanophenyl)-6-hexylquinolin-2-yl](methyl)amino}acetic acid.

Synthetic method B:

Representative example: 2{[4-(3-cyanophenyl)-6-hexylquinolin-2-yl]oxy}acetic acid

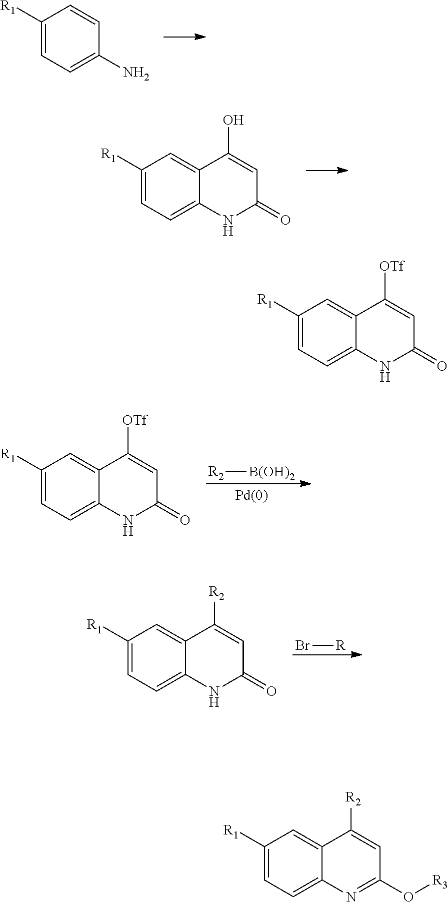

4-hexyl aniline (5 g, 28.24 mmol) in CH₂Cl₂ (50 mL) was cooled to 0° C. and then diethyl malonate 2 was added at stirred for 2 h at room temperature. Reaction mixture was quenched with ice and neutralized with saturated NaHCO₃ solution. Resulting solution was then extracted with CH₂Cl₂ (×2) then dried over Na₂SO₄ and concentrated. 5 g of desired ester amide product was obtained by purification by flash column chromatography which was taken to next step.

5 g of ester amide in PolyPhosphoricAcid (50 mL) was heated to 150° Ch and stirred. After 4 h, the reaction was poured into ice and stirred. Pale brown solid was formed after 30 minutes which was filtered and dried to yield 3.5 g of the desired bicyclic product, as determined by LCMS and HNMR.

Bicyclic beta-keto amide (3.5 g, 14.285 mmol) in 15 mL of DMF was cooled to 0° C. and triethylamine (2.48 mL, 17.142 mmol) was added and stirred for 5 min. Comin's reagent (3.78 g, 17.142 mmol) was added and the reaction mixture was stirred for 2 h at room temperature. Reaction mixture was then poured into ice and pale yellow solid was formed which was filtered and dried to yield 3.0 g of the desired product, as determined by LCMS and HNMR.

Bicyclic triflate (1.0 g, 2.652 mmol), 3-cyano-phenyl boronic acid (0.580 g, 3.978 mmol) and Na₂CO₃ (0.562 g, 5.304 mmol) was added in DMF+H₂O (10 mL: 2 mL) solution. Argon gas was bubbled and Pd(dppf)C12(0.193 g, 0.265 mmol) was added at which point the reaction was moved to pre-heated 100° C. oil bath and stirred for 1h. Reaction mixture was diluted with water, resulting in formation of brown precipitation which was then filtered and dried to yield the desired product, as determined by LCMS and HNMR.

Tricyclic amide (0.4 g, 1.212 mmol) in DMF (5 mL) was cooled to 0° C. and NaH (60% in Mineral Oil, 0.145 g, 3.636 mmol) was added portion wise and stirred for 30 minutes. Bromo-methyl acetate (0.1303 g, 1.818 mmol) was added at 0° C. and the reaction mixture, after removal of ice bath, was stirred for 4 h. Reaction was quenched with ice and organic layer was extracted with EtOAc (×2), dried over Na₂SO₄ and concentrated by flash column chromatography gave the desired bromide product as determined by LCMS and HNMR.

Cyano ester bromide (0.17 g, 0.4096 mmol) in EtOH:H₂O (5 mL:1 mL) was added KOH (0.07 g, 1.2289 mmol) and the reaction mixture was then stirred for 2 h at room temperature. After evaporating EtOH, crude reaction was diluted with 1 mL of H₂O and pH adjusted to 4 with 1N HCl, pale yellow precipitation was formed which was dried and washed with Et2O and n-pentane to yield 2-{[4-(3-cyanophenyl)-6-hexylquinolin-2-yl]oxy}acetic acid, as determined by LCMS and HNMR.

Synthetic method C:

Representative example: 2-{[6-hexyl-4-(morpholin-4-yl)quinolin-2-yl](methyl)amino}acetic acid

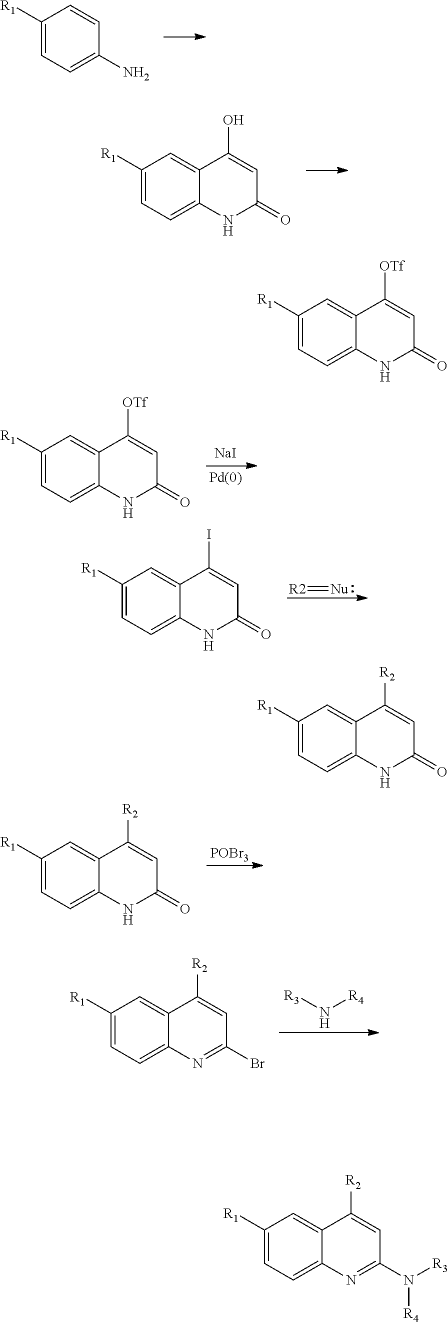

4-hexyl aniline (5 g, 28.24 mmol) in CH₂Cl₂(50 mL) was cooled to 0° C. and then 2 was added at stirred for 2 h at room temperature. Reaction mixture was quenched with ice and neutralized with saturated NaHCO₃ solution. Resulting solution was then extracted with CH₂Cl₂ (×2) then dried over Na₂SO₄ and concentrated. 5 g of desired ester amide product was obtained by purification by flash column chromatography which was taken to next step. 5 g of ester amide in PolyPhosphoricAcid (50 mL) was heated to 150° C. and stirred. After 4 h, the reaction was poured into ice and stirred. Pale brown solid was formed after 30 minutes which was filtered and dried to yield 3.5 g of the desired bicyclic product, as determined by LCMS and HNMR.

Bicyclic beta-keto amide(3.5 g, 14.285 mmol) in 15 mL of DMF was cooled to 0° C. and triethylamine (2.48 mL, 17.142 mmol) was added and stirred for 5 min. Comin's reagent (3.78 g, 17.142 mmol) was added and the reaction mixture was stirred for 2 h at room temperature. Reaction mixture was then poured into ice and pale yellow solid was formed which was filtered and dried to yield 3.0 g of the desired product , as determined by LCMS and HNMR.

To bicyclic triflate (0.5 g, 1.3262 mmol), in CH₃CN(10 mL) was added conc. HCl (0.1 mL, 1.452 mmol) followed by Sodium Iodide (1.989 g, 13.262 mmol) at 0° C. and stirred at room temperature for 16 h. Reaction mixture was diluted with water and extracted with EtOAc (×2), dried over Na₂SO₄ and concentrated. Purification by flash column chromatography yielded the desired product, as determined by LCMS and HNMR.

Iodo amide (0.2 g, 0.5633 mmol) in NMP (1 mL) was added morpholine (490mg, 5.633 mmol) and triethylamine (0.4 mL, 02.816 mmol). The reaction mixture was stirred for 1h at 160° C. Reaction was diluted with water and organic layer was extracted with EtOAc(×2), dried over Na₂SO₄ and concentrated by flash column chromatography gave the desired bromide product as determined by LCMS and HNMR.

Morpholino amide (0.1 g, 0.3184 mmol) in toluene (3 mL) was added POBr₃ (0.273 g, 0.955 mmol) and stirred for 3 h at 140° C. The reaction mixture was poured into ice and the aqueous layer was extract with EtOAc (×2). The organic layer was then dried over Na₂SO₄ and concentrated. Purification by flash column chromatography gave the desired bromide product as determined by LCMS and HNMR.

Morpholino bromide (0.08 g, 0.212 mmol) in DMSO (3 mL) was added N-Methyl glycine methyl ester (0.145 g, 1.063 mmol) and K₂CO₃ (0.090 g, 0.636 mmol). Reaction mixture was then stirred at 100° C. for 16 h before quenched with H₂O. The mixture was acidified with 1N HCl to adjust the pH-6 before being extracted with EtOAc (×2). Organic layer was dried over Na₂SO₄ and concentrated before purification by reverse phase prep-HPLC to yield 2-{[6-hexyl-4-(morpholin-4-yl)quinolin-2-yl](methyl)amino}acetic acid as confirmed by H-NMR and LCMS.

Additional information regarding the synthetic methods finds support in PCT/US21/14250 filed on Jan. 20, 2021, the entire contents of which are hereby incorporated by reference in their entirety.

Technical outcomes of the above are reflected below in Table 1.

TABLE 1

| | | LCMS method/rt | |
|---|---|---|---|
| | | Observed mass | Synthetic |
| IUPAC Name | NMR | Exact mass | Method |
| 3-{[6-butyl-4-(4- | 400 MHz-DMSO-d6: 12.4 (s, | LCMS5.5/2.09 | method A: R1 = |
| fluorophenyl)quinol | 1H), 7.58-7.54 (m, 3H), 7.42- | Observed mass: | n-Butyl; |
| in-2- | 7.37(m, 3H), 7.27(d, J = | Exact mass: 394.21 | R2 = 4-Fluoro- |
| yl](methyl)amino}- | 1.6 Hz, 1H), 6.90(s, 1H), | | Phenyl; R3 = 2- |
| 2-methylpropanoic | 3.79(d, J = 7.2 Hz, 2H), 3.15 | | methyl- |
| acid | (s, 3H), 2.91 (q, J = 10.4, 7.2 | | propionic acid; |
| | Hz, 1H), 2.58(t, J = 7.2 Hz, | | R4 = methyl |
| | 2H), 1.57-1.49(m, 2H), 1.30- | | |
| | 1.23(m, 4m), 1.09(d, J = | | |
| | 7.6 Hz, 3H), 0.83(t, J = 6.8 Hz, | | |
| | 3 Hz) | | |
| 3-{[4-(4- | 400 MHz-DMSO-d6: 12.4 (s, | LCMS5.5/2.16 | method A: R1 = |
| fluorophenyl)-6- | 1H), 7.57-7.54 (m, 3H), 7.41- | Observed mass [M − H]: | n-hexyl; |
| hexylquinolin-2- | 7.37(m, 3H), 7.27(s, 1H), | 421.24 | R2 = 4-Fluoro- |
| yl](methyl)amino}- | 6.90(s, 1H), 3.79 (d, J = | Exact mass: 422.24 | Phenyl; R3 = 2- |
| 2-methylpropanoic | 7.6 Hz, 2H), 3.15 (s, 3H), 2.90 | | methyl- |
| acid | (q, J = 14.4, 6.8 Hz, 1H), | | propionic acid; |
| | 2.57(t, J = 7.6 Hz, 2H), 1.57- | | R4 = methyl |
| | 1.49(m, 2H), 1.30-1.23(m, | | |
| | 6m), 1.08 (d, J = 7.6 Hz, 3H), | | |
| | 0.82(t, J = 6.8 Hz, 3 Hz) | | |
| 2-{[4-(4- | 400 MHz-DMSO-d6: 12.4 (s, | LCMS5.5/2.11 | method A: R1 = |
| fluorophenyl)-6- | 1H), 7.57-7.54 (m, 3H), 7.45- | Observed mass [M − H]: | n-pentyl; |
| pentylquinolin-2- | 7.37(m, 3H), 7.27(s, 1H), | 379.29 | R2 = 4-Fluoro- |
| yl](methyl)amino}acetic | 6.90(br s, 1H), 4.49(s, 2H), | Exact mass: 380.19 | Phenyl; |
| acid | 3.25-3.15 (m, 3H), 2.80- | | R3 = ethyl |
| | 2.57(m, 2H), 1.57-1.49(m, | | carboxylic acid; |
| | 2H), 1.30-1.22(m, 4H), 0.82(t, | | R4 = methyl |
| | J = 6.8 Hz, 3 Hz) | | |
| 2-{[4-(4- | 400 MHz-DMSO-d6: 12.4 (s, | LCMS5.5/2.19 | method A: R1 = |
| fluorophenyl)-6- | 1H), 7.57-7.54 (m, 3H), 7.45- | Observed mass [M − H]: | n-hexyl; |
| hexylquinolin-2- | 7.38(m, 3H), 7.30(s, 1H), | 393.38 | R2 = 4-Fluoro- |
| yl](methyl)amino}acetic | 6.97(br s, 1H), 4.47(s, 2H), | Exact mass: 394.21 | Phenyl; |
| acid | 3.25-3.16 (m, 3H), 2.67- | | R3 = ethyl |
| | 2.57(m, 2H), 1.57-1.49(m, | | carboxylic acid; |
| | 2H), 1.30-1.22(m, 6H), 0.82(t, | | R4 = methyl |
| | J = 6.0 Hz, 3 Hz) | | |
| 2-{[6-hexyl-3- | 500 MHz-DMSO-d6: 12.4 (s, | LCMS_5.5 min/2.13 | |
| methyl-4- | 1H), 7.72 (s, 1H), 7.49 (d, J = | Observed mass [M + H]: | |
| (morpholin-4- | 8.4 Hz, 1H), 7.34 (dd, J = 8.4, | 400.50 | |
| yl)quinolin-2- | 2 Hz, 1H), 3.96 (s, 2H), | Exact mass: 399.25 | |
| yl](methyl)amino}acetic | 3.83(m, 4H), 3.20 (m, 4H), | | |
| acid | 2.96 (s, 3H), 2.72 (t, J = | | |
| | 7.2 Hz, 2H), 2.27 (s, 3H), | | |
| | 1.63-1.55(m, 2H), 1.35- | | |
| | 1.26(m, 8H), 0.85 (t, J = | | |
| | 7.2 Hz, 3H) | | |
| 2-{[4-(4- | 500 MHz-DMSO-d6: 12.4 (s, | RND-FA-4.51/2.491 | method A″′: |
| fluorophenyl)-6- | 1H), 7.60 (d, J = 7.5 Hz, 1H), | Observed mass [M + H]: | R1 = n-hexyl; |
| hexyl-3- | 7.42-7.38(m, 3H), 7.36- | 409.10 | R1′ = H, |
| methylquinolin-2- | 7.33(m, 2H), 6.87(br s, 1H), | Exact mass: 408.22 | R1″ = |
| yl](methyl)amino}acetic | 4.01(s, 2H), 3.04(s, 3H), 2.67- | | Methyl |
| acid | 2.57(m, 2H), 2.07 (s, 3H), | | R2 = 4-Fluoro- |
| | 1.49-1.47(m, 2H), 1.24- | | Phenyl; |
| | 1.20(m, 8H), 0.81(t, J = | | R3 = ethyl |
| | 6.0 Hz, 3 Hz) | | carboxylic acid; |
| | | | R4 = methyl |
| 2-{[4,6-bis(4- | 400 MHz-DMSO-d6: 12.4 (s, | LCMS_5 MIN/2.72 | method A: R1 = |
| fluorophenyl)quinolin-2- | 1H), 7.83 (dd, J = 8.0, 2 Hz, | Observed mass [M + H]: | 4-Fluoro- |
| yl](methyl)amino}acetic | 1H), 7.69-7.59(m, 6H), 7.04 | 405.30 | Phenyl; |
| acid | (t, J − 9.2 Hz, 2H), 7.25 (t, | Exact mass: 404.13 | R2 = 4-Fluoro- |
| | J − 9.2 Hz, 2H), 6.97(br s, 1H), | | Phenyl; |
| | 4.44(s, 2H), 3.226 (s, 3H) | | R3 = ethyl |
| | | | carboxylic acid; |
| | | | R4 = methyl |
| 2-{[4-(4- | 400 MHz-DMSO-d6: 12.4 (s, | LCMS_5 MIN/3.13 | method A: R1 = |
| fluorophenyl)-6- | 1H), 7.55-7.49 (m, 3H), 7.41- | Observed mass [M + H]: | n-hexyl; |
| hexylquinolin-2- | 7.37(m, 3H), 7.26(s, 1H), | 437.46 | R2 = 4-Fluoro- |
| yl](2- | 6.88(s, 1H), 4.32 (s, 2H), 3.42 | Exact mass: 436.25 | Phenyl; |
| methylpropyl)amino}acetic | (d, J = 7.6 Hz, 2H), 3.15 (s, | | R3 = ethyl |
| acid | 3H), 2.58 (t, J = 7.2 Hz, 2H), | | carboxylic |
| | 2.03 (m, 1H), 1.52(m, 2H), | | acid; R4 = 2- |
| | 1.30-1.23(m, 6H), 0.92 (d, J = | | methyl-propyl |
| | 6.4 Hz, 6H), 0.82(t, J = 6.8 Hz, | | |
| | 3 Hz) | | |
| 2-{[4-(4- | 400 MHz-DMSO-d6: 12.4 (s, | LCMS_5 MIN/3.00 | method A: R1 = |
| fluorophenyl)-6- | 1H), 7.55-7.49 (m, 3H), 7.41- | Observed mass [M − H]: | n-hexyl; |
| hexylquinolin-2- | 7.37(m, 3H), 7.26(s, 1H), | 421.35 | R2 = 4-Fluoro- |
| yl](propyl)amino}acetic | 6.86(s, 1H), 4.33 (s, 2H), 3.56 | Exact mass: 422.24 | Phenyl; |
| acid | (t, J = 7.2 Hz, 2H), 2.58 | | R3 = ethyl |
| | (m, 2H), 1.66-1.62(m, 2H), | | carboxylic |
| | 1.52(m, 2H), 1.30-1.23(m, | | acid; R4 = |
| | 6H), 0.90 (t, J = 7.6 Hz, 3H), | | n-propyl |
| | 0.82(t, J = 6.8 Hz, 3 Hz) | | |
| 2-{[4-(4- | | Observed mass: | method A: R1 = |
| fluorophenyl)-6- | | Exact mass: 380.19 | n-hexyl; |
| hexylquinolin-2- | | | R2 = 4-Fluoro- |
| yl]amino} acetic | | | Phenyl; |
| acid | | | R3 = ethyl |
| | | | carboxylic acid; |
| | | | R4 = hydrogen |
| 2-{ethyl[4-(4- | 400 MHz-DMSO-d6: 12.4 (s, | LCMS_5.5/2.25 | method A: R1 = |
| fluorophenyl)-6- | 1H), 7.55-7.50 (m, 3H), 7.41- | Observed mass [M + H]: | n-hexyl; |
| hexylquinolin-2- | 7.37(m, 3H), 7.27(s, 1H), | 409.48 | R2 = 4-Fluoro- |
| yl]amino}acetic | 6.85(s, 1H), 4.33 (s, 2H), 3.66 | Exact mass: 408.22 | Phenyl; |
| acid | (m, 2H), 2.59-2.54 (m, 2H), | | R3 = ethyl |
| | 1.52-1.50 (m, 2H), 1.30- | | carboxylic |
| | 1.23(m, 8H), 1.15 (t, J = | | acid; R4 = |
| | 6.8 Hz, 3H), 0.82(t, J = 6.8 Hz, | | n-ethyl |
| | 3H) | | |
| 2-{[6-hexyl-4- | 400 MHz-DMSO-d6: 12.4 (s, | LCMS_5 min/2.71 | method C: R1 = |
| (pyridin-3- | 1H), 8.55(d, J = 2.4 Hz, 1H), | Observed mass [M + H]: | n-hexyl; |
| yloxy)quinolin-2- | 8.50(d, J = 4.0 Hz, 1H), | 394.38 | R2 = 3- |
| yl](methyl)amino}acetic | 7.70(s, 1H), 7.67-7.64(m, | Exact mass: 393.21 | hydroxy- |
| acid | 1H), 7.53-7.43 (m, 3H), | | pyridyl; |
| | 6.22(s, 1H), 4.21 (s, 2H), | | R3 = ethyl |
| | 3.03(s, 3H), 2.67(t, J = 7.6 Hz, | | carboxylic acid; |
| | 2H), 1.62-1.58 (m, 2H), 1.33- | | R4 = methyl |
| | 1.20(m, 6H), 0.84(t, J = | | |
| | 6.8 Hz, 3H) | | |
| 2-{[6-hexyl-4- | 400 MHz-DMSO-d6: 12.4 (s, | RND X-bridge 5.0min/ | method C: R1 = |
| (pyridin-4- | 1H), 7.83(d, J = 8 Hz, 2H), | 1.949 | n-hexyl; |
| yloxy)quinolin-2- | 7.54 (d, J = 8.8 Hz, 1H), | Observed mass: | R2 = 4- |
| yl](methyl)amino}acetic | 7.43(d, J = 8.8 Hz, 1H), 7.06 | Exact mass: 393.21 | hydroxy- |
| acid | (s, 1H), 7.02 (s, 1H), 6.26 d, | | pyridyl; |
| | J = 8 Hz, 2H), 4.05 (s, 2H), | | R3 = ethyl |
| | 3.16(s, 3H), 2.62(t, J = 7.6 Hz, | | carboxylic acid; |
| | 2H), 1.57-1.53 (m, 2H), 1.33- | | R4 = methyl |
| | 1.20(m, 6H), 0.84 (t, J = | | |
| | 6.8 Hz, 3H) | | |
| 2-{[4-(3- | 400 MHz-DMSO-d6: 12.4 (s, | LCMS_5.5 min/2.21 | method C: R1 = |
| fluorophenoxy)-6- | 1H), 7.61(s, 1H), 7.52- | Observed mass [M − H]: | n-hexyl; |
| hexylquinolin-2- | 7.46(m, 2H), 7.43-7.40 (m, | 409.22 | R2 = 3-fluoro- |
| yl](methyl)amino}acetic | 1H), 7.11-7.07(m, 2H), 7.01- | Exact mass: 410.20 | 1-hydroxy- |
| acid | 6.99 (m, 1H), 6.3(s, 1H), 4.15 | | phenyl; |
| | (s, 2H), 3.02(s, 3H), 2.65(t, J = | | R3 = ethyl |
| | 7.6 Hz, 2H), 1.62-1.56 (m, | | carboxylic acid; |
| | 2H), 1.33-1.18(m, 6H), 0.83(t, | | R4 = methyl |
| | J = 6.8 Hz, 3H) | | |
| 2-{[4-(4- | 400 MHz-DMSO-d6: 12.4 (s, | LCMS_5 min/2.87 | method C: R1 = |
| fluorophenoxy)-6- | 1H), 7.71(s, 1H), 7.48- | Observed mass [M + H]: | n-hexyl; |
| hexylquinolin-2- | 7.40(m, 2H), 7.34-7.24 (m, | 411.38 | R2 = 4-fluoro- |
| yl](methyl)amino}acetic | 4H), 6.08(s, 1H), 4.16 (s, 2H), | Exact mass: 410.20 | 1-hydroxy- |
| acid | 2.97(s, 3H), 2.67(t, J = 7.6 Hz, | | phenyl; |
| | 2H), 1.62-1.56 (m, 2H), 1.33- | | R3 = ethyl |
| | 1.18(m, 6H), 0.84(t, J = | | carboxylic acid; |
| | 6.8 Hz, 3H) | | R4 = methyl |
| 2-{[4-(4- | 400 MHz-DMSO-d6-D2O: | LCMS_5 min/3.34 | method A: R1 = |
| fluorophenyl)-6- | 12.4 (s, 1H), 7.74 (m, 1H), | Observed mass [M + H]: | n-octyl; |
| octylquinolin-2-yl](2- | 7.58-7.56 (m, 3H), 7.43(t, J = | 465.53 | R2 = 4-Fluoro- |
| methylpropyl)amino}acetic | 7.2 Hz, 2H), 7.31(s, 1H), | Exact mass: 464.28 | Phenyl; |
| acid | 7.06(s, 1H), 4.56 (s, 2H), 3.66 | | R3 = ethyl |
| | (m, 2H), 2.66-2.59 (m, 2H), | | carboxylic |
| | 2.05-2.01 (m, 2H), 1.62- | | acid; R4 = |
| | 1.50(m, 2H), 1.23-1.20(m, | | 2-methyl-propyl |
| | 12H), 0.94 (d, J = 7.5 Hz, 6H), | | |
| | 0.83 (t, J = 7.0 Hz, 3H) | | |
| 2-{[4-(4- | 400 MHz-DMSO-d6: 12.4 (s, | LCMS_5 min/3.16 | method A: R1 = |
| fluorophenyl)-6- | 1H), 7.54-7.49 (m, 3H), 7.47- | Observed mass [M + H]: | n-octyl; |
| octylquinolin-2- | 7.35(m, 3H), 7.24(s, 1H), | 451.53 | R2 = 4-Fluoro- |
| yl](propyl)amino}acetic | 6.84(s, 1H), 4.32 (s, 2H), 3.54 | Exact mass: 450.27 | Phenyl; |
| acid | (m, 2H), 2.57-2.54 (m, 2H), | | R3 = ethyl |
| | 1.62-1.50(m, 2H), 1.53- | | carboxylic |
| | 1.49(m, 2H), 1.30-1.20(m, | | acid; R4 = |
| | 10H), 0.88 (t, J = 7.6 Hz, 6H), | | n-propyl |
| | 0.82 (t, J = 7.2 Hz, 3H) | | |
| 2-{ethyl[4-(4- | 400 MHz-DMSO-d6: 12.4 (s, | RND_FA_4.01/2.37 | method A: R1 = |
| fluorophenyl)-6- | 1H), 7.56-7.50 (m, 3H), 7.41- | Observed mass [M + H]: | n-octyl; |
| octylquinolin-2- | 7.37(m, 3H), 7.26(s, 1H), | 437.10 | R2 = 4-Fluoro- |
| yl]amino}acetic | 6.85(s, 1H), 4.32 (s, 2H), | Exact mass: 436.25 | Phenyl; |
| acid | 3.68-3.64 (m, 2H), 2.57-2.54 | | R3 = ethyl |
| | (m, 2H), 1.55-1.50(m, 2H), | | carboxylic |
| | 1.53-1.49(m, 2H), 1.30- | | acid; R4 = ethyl |
| | 1.20(m, 10H), 1.17 (t, J = | | |
| | 7.2 Hz, 3H), 0.83 (t, J = 7.2 Hz, | | |
| | 3H) | | |
| 2-{methyl[6-octyl- | 400 MHz-DMSO-d6: 12.4 (s, | LCMS_5 min/2.84 | method C: R1 = |
| 4-(pyridin-3- | 1H), 8.56(d, J = 2.4 Hz, 1H), | Observed mass [M + H]: | n-octyl; |
| yloxy)quinolin-2- | 8.50(d, J = 4.0 Hz, 1H), | 422.44 | R2 = 3- |
| yl]amino}acetic | 7.71(s, 1H), 7.66-7.65(m, | Exact mass: 421.24 | hydroxy- |
| acid | 1H), 7.53-7.46 (m, 3H), | | pyridyl; |
| | 6.22(s, 1H), 4.31 (s, 2H), | | R3 = ethyl |
| | 3.00(s, 3H), 2.67(t, J = 7.6 Hz, | | carboxylic acid; |
| | 2H), 1.62-1.58 (m, 2H), 1.33- | | R4 = methyl |
| | 1.15(m, 10H), 0.84 (t, J = | | |
| | 6.8 Hz, 3H) | | |
| 2-{methyl[6-octyl- | 400 MHz-DMSO-d6: 12.4 (s, | LCMS_5 min/2.86 | method C: R1 = |
| 4-(pyridin-4- | 1H), 7.81(d, J = 8 Hz, 2H), | Observed mass [M + H]: | n-octyl; |
| yloxy)quinolin-2- | 7.54 (d, J = 8.8 Hz, 1H), | 422.39 | R2 = |
| yl]amino}acetic | 7.43(d, J = 8 Hz, 1H), 7.06 (s, | Exact mass: 421.24 | 4-hydroxy- |
| acid | 1H), 7.00 (s, 1H), 6.26 (d, J = | | pyridyl; |
| | 8 Hz, 2H), 3.99 (br s, 2H), | | R3 = ethyl |
| | 3.16(s, 3H), 2.63(t, J = 7.6 Hz, | | carboxylic acid; |
| | 2H), 1.57-1.53 (m, 2H), 1.33- | | R4 = methyl |
| | 1.15(m, 10H), 0.84 (t, J = | | |
| | 6.8 Hz, 3H) | | |
| 2-{[4-(3- | 400 MHz-DMSO-d6: 12.4 (s, | LCMS_5.5nin/2.39 | method C: R1 = |
| fluorophenoxy)-6- | 1H), 7.64(s, 1H), 7.53- | Observed mass [M − H]: | n-octyl; |
| octylquinolin-2- | 7.47(m, 2H), 7.45-7.42 (m, | 437.28 | R2 = 3-fluoro- |
| yl](methyl)amino}acetic | 1H), 7.13-7.09(m, 2H), 7.03- | Exact mass: 438.23 | 1-hydroxy- |
| acid | 7.01 (m, 1H), 6.32(s, 1H), | | phenyl; |
| | 4.30 (s, 2H), 3.02(s, 3H), | | R3 = ethyl |
| | 2.66(t, J = 7.6 Hz, 2H), 1.62- | | carboxylic |
| | 1.56 (m, 2H), 1.33-1.18(m, | | acid; |
| | 10H), 0.84 (t, J = 6.8 Hz, 3H) | | R4 = methyl |
| 2-{[4-(4- | 400 MHz-DMSO-d6: 12.4 (s, | LCMS_5 min/2.99 | method C: R1 = |
| fluorophenoxy)-6- | 1H), 7.74(s, 1H), 7.46(d, J = | Observed mass [M + H]: | n-octyl; |
| octylquinolin-2- | 8.8 Hz, 1H), 7.44(d, J = 8.8 Hz, | 439.45 | R2 = 4-fluoro- |
| yl](methyl)amino}acetic | 1H), 7.35-7.26(m, 3H), 6.09 | Exact mass: 438.23 | 1-hydroxy-phenyl; |
| acid | (s, 1H), 4.29 (s, 2H), 2.97(s, | | R3 = ethyl |
| | 3H), 2.67(t, J = 7.6 Hz, 2H), | | carboxylic acid; |
| | 1.65-1.53 (m, 2H), 1.33- | | R4 = methyl |
| | 1.20(m, 10H), 0.84 (t, J = | | |
| | 6.8 Hz, 3H) | | |
| 2-{[6-decyl-4-(4- | 400 MHz-DMSO-d6: 12.4 (s, | AA_9.0/4.1 | method A: R1 = |
| fluorophenyl)quinolin-2- | 1H), 7.56-7.53 (m, 3H), 7.41- | Observed mass [M + H]: | n-decyl; |
| yl](methyl)amino}acetic | 7.36(m, 3H), 7.28 (s, 1H), | 451.51 | R2 = 4-Fluoro- |
| acid | 6.89(br s, 1H), 4.39(s, | Exact mass: 450.27 | Phenyl; |
| | 2H), 3.16(s, 3H), 2.67-2.57(m, | | R3 = ethyl |
| | 2H), 1.57-1.49(m, 2H), 1.30- | | carboxylic |
| | 1.22(m, 14H), 0.82(t, J = | | acid; |
| | 6.0 Hz, 3 Hz) | | R4 = methyl |
| 2-{[4-(4- | 400 MHz-DMSO-d6: 12.4 (s, | LCMS_5.5/2.28 | method A: R1 = |
| fluorophenyl)-6- | 1H), 7.56-7.52 (m, 3H), 7.41- | Observed mass [M + H]: | n-septyl; |
| heptylquinolin-2- | 7.36 (m, 3H), 7.28 (s, 1H), | 409.47 | R2 = 4-Fluoro- |
| yl](methyl)amino}acetic | 6.89 (s, 1H), 4.35 (s, 2H), | Exact mass: 408.22 | Phenyl; |
| acid | 3.04 (s, 3H), 2.57-2.54 | | R3 = ethyl |
| | (m, 2H), 1.55-1.50(m, 2H), | | carboxylic |
| | 1.30-1.20(m, 8H), 0.83 (t, J = | | acid; |
| | 7.2 Hz, 3H) | | R4 = methyl |
| 2-{[4-(4- | 400 MHz-DMSO-d6: 12.4 (s, | LCMS 5.0/3.02 | method A: R1 = |
| fluorophenyl)-6- | 1H), 7.57-7.53 (m, 3H), 7.41- | Observed mass [M + H]: | n-octyl; |
| octylquinolin-2- | 7.37 (m, 3H), 7.28 (s, 1H), | 423.44 | R2 = 4-Fluoro- |
| yl](methyl)amino}acetic | 6.91 (s, 1H), 4.41 (s, 2H), | Exact mass: 422.24 | Phenyl; |
| acid | 3.19 (s, 3H), 2.60-2.54 | | R3 = ethyl |
| | (m, 2H), 1.55-1.50(m, 2H), | | carboxylic |
| | 1.30-1.20(m, 10H), 0.83 (t, | | acid; |
| | J = 9.6 Hz, 3H) | | R4 = methyl |
| 2-[(6- | | Observed mass: | |
| hexylquinolin-2- | | Exact mass: 300.18 | |
| yl)(methyl)amino]acetic | | | |
| acid | | | |
| 2-{2- | 400 MHz-DMSO-d6: 12.5 (s, | RND_FA3.5/1.815 | method A: R1 = |
| [(carboxymethyl)(methyl)amino]- | 2H), 7.97 (dd, J = 8.0, 1.2 Hz, | Observed mass [M + H]: | n-hexyl; |
| 6-hexylquinolin-4- | 1H), 7.69 (dt, J = 7.6, 1.2 Hz, | 421.00 | R2 = 2- |
| yl}benzoic acid | 1H), 7.61 (dt, J = 7.6, 1.2 Hz, | Exact mass: 420.20 | carboxylic |
| | 1H), 7.49 (d, J = 8.4 Hz, 1H), | | acid-Phenyl; |
| | 7.37-7.32 (m, 2H), 6.91 (s, | | R3 = ethyl |
| | 1H), 6.85 (s, 1H), 4.45 (d, J = | | carboxylic |
| | 18 Hz, 1H), 4.35 (d, J = | | acid; |
| | 18 Hz, 1H), 3.16 (s, 3H), | | R4 = methyl |
| | 1.52-1.40(m, 2H), 1.30- | | |
| | 1.20(m, 6H), 0.81 (t, J = | | |
| | 6.4 Hz, 3H) | | |
| 2-{[4-(4,4- | 400 MHz-DMSO-d6: 12.4 (s, | LCMS_5 min/2.79 | method C: R1 = |
| difluoropiperidin-1- | 1H), 7.52 (s, 1H), 7.41 (d, J = | Observed mass [M + H]: | n-hexyl; |
| yl)-6-hexylquinolin- | 8.4 Hz, 1H), 7.31 (dd, J = 8.4, | 420.44 | R2 = 4,4′- |
| 2-yl](methyl)amino}acetic | 2 Hz, 1H), 6.46 (s, 1H), 4.29 | Exact mass: 419.24 | di-fluoro- |
| acid | (s, 2H), 3.30-3.17(m, 4H), | | piperidine; |
| | 3.14 (s, 3H), 2.68 (t, J = | | R3 = ethyl |
| | 7.2 Hz, 2H), 2.33-2.21(m, | | carboxylic acid; |
| | 4H), 1.65-1.58(m, 2H), 1.35- | | R4 = methyl |
| | 1.26(m, 6H), 0.86 (t, J = | | |
| | 7.2 Hz, 3H) | | |
| 2-{[4-(3,3- | 400 MHz-DMSO-d6: 12.4 (s, | LCMS_5 min/2.75 | method C: R1 = |
| difluoropyrrolidin- | 1H), 7.62 (s, 1H), 7.38 (d, J = | Observed mass [M + H]: | n-hexyl; |
| 1-yl)-6- | 8.4 Hz, 1H), 7.29 (dd, J = 8.4, | 406.40 | R2 = 3,3′-di- |
| hexylquinolin-2- | 2 Hz, 1H), 6.20 (s, 1H), 4.33 | Exact mass: 405.22 | fluoro- |
| yl](methyl)amino}acetic | (s, 2H), 3.88(t, J = 13.2 Hz, | | pyrrolidine; |
| acid | 2H), 3.66 (t, J = 6.8 Hz, 2H), | | R3 = ethyl |
| | 3.17 (s, 3H), 2.67 (t, J = | | carboxylic acid; |
| | 7.2 Hz, 2H), 2.59-2.50(m, | | R4 = methyl |
| | 2H), 1.63-1.55(m, 2H), 1.32- | | |
| | 1.26(m, 6H), 0.85 (t, J = | | |
| | 7.2 Hz, 3H) | | |
| 2-{[6-hexyl-4- | 400 MHz-DMSO-d6: 12.4 (s, | LCMS_5 min/2.67 | method C: R1 = |
| (morpholin-4- | 1H), 7.50 (s, 1H), 7.40 (d, J = | Observed mass [M + H]: | n-hexyl; |
| yl)quinolin-2- | 8.4 Hz, 1H), 7.40 (dd, J = 8.4, | 386.10 | R2 = N- |
| yl](methyl)amino}acetic | 2 Hz, 1H), 6.39 (s, 1H), 4.24 | Exact mass: 385.24 | morpholine; |
| acid | (s, 2H), 3.86(m, 4H), 3.14 (s, | | R3 = ethyl |
| | 3H), 3.09(m, 4H), 2.66 (t, J = | | carboxylic acid; |
| | 7.2 Hz, 2H), 1.63-1.55(m, | | R4 = methyl |
| | 2H), 1.35-1.26(m, 6H), 0.85 | | |
| | (t, J = 7.2 Hz, 3H) | | |
| 2-{[6-butyl-4-(2- | 400 MHz-DMSO-d6: 12.5(s, | LCMS_5 MIN/1.92 | method A: R1 = |
| methyl-pyridin-4- | 1H), 8.62(s, 1H), 8.55(d, J = | Observed mass [M + H]: | n-hexyl; |
| yl)quinolin-2- | 4 Hz, 1HH), 7.55(d, J = | 392.25 | R2 = 4-(2- |
| yl](methyl)amino}acetic | 8.8 Hz, 1H), 7.40 (d, J = | Exact mass: 391.23 | methyl)pyridyl; |
| acid | 8.0 Hz, 2H), 7.26(d, J = 4 Hz, | | R3 = ethyl |
| | 1H), 6.90 (s, 1H), 6.81(s, | | acid; |
| | 1H), 4.42-4.35 (m, 2H), 3.17 | | R4 = methyl |
| | (s, 3H), 2.62 (t, J = 8 Hz, 2H), | | |
| | 2.01(s, 3H), 1.58-1.51 (m, | | |
| | 2H), 1.35-1.26(m, 6H), 0.90 | | |
| | (t, J = 6.5 Hz, 3H) | | |
| 2-{[4-(3,5- | 400 MHz-DMSO-d6: 12.4 (s, | LCMS_5 min/2.76 | method A: R1 = |
| dimethyl-1,2- | 1H), 7.50 (d, J = 8.4 Hz, 1H), | Observed mass [M + H]: | n-hexyl; R2 = 3,5- |
| oxazol-4-yl)-6- | 7.36 (dd, J = 8.8, 1.6 Hz, 1H), | 396.42 | methyl-4-oxazole; |
| hexylquinolin-2- | 7.03 (s, 1H), 6.81 (s, 1H), | Exact mass: 395.22 | R3 = ethyl |
| yl](methyl)amino}acetic | 4.04 (s, 2H), 3.15 (s, 3H), | | carboxylic acid; |
| acid | 2.60 (t, J = 7.2 Hz, 2H), | | R4 = methyl |
| | 2.28(s, 3H), 2.06(s, 3H), 1.55- | | |
| | 1.50(m, 2H), 1.30-1.20(m, | | |
| | 6H), 0.83 (t, J = 7.2 Hz, 3H) | | |
| 2-{[4-(3- | 500 MHz-DMSO-d6: 12.5 (s, | RND X-bridge 5.0 / | method A: R1 = |
| cyanophenyl)-6- | 1H), 7.99-7.96 (m, 2H), 7.81 | 2.369 | n-hexyl; |
| hexylquinolin-2- | (d, J = 6.0 Hz, 1H), 7.75(t, J = | Observed mass [M + H]: | R2 = 3-cyano- |
| yl](methyl)amino}acetic | 8 Hz, 1H), 7.52 (d, J = 8.5 Hz, | 401.90 | Phenyl; |
| acid | 1H), 7.38 (d, J = 8.5 Hz, 1H), | Exact mass: 401.21 | R3 = ethyl |
| | 7.18 (s, 1H), 6.87 (s, 1H), | | carboxylic acid; |
| | 4.17 (s, 1H), 3.18 (s, 3H), | | R4 = methyl |
| | 2.57 (m, 2H), 1.52-1.40(m, | | |
| | 2H), 1.52-1.50(m, 2H), 1.23 | | |
| | (m, 6H), 0.81 (t, J = 6.4 Hz, | | |
| | 3H) | | |
| 3-{[4-(3- | 400 MHz-DMSO-d6: 12.5 (s, | LCMS_5 min/2.79 | method A: R1 = |
| cyanophenyl)-6- | 1H), 7.99-7.96 (m, 2H), 7.83 | Observed mass [M + H]: | n-hexyl; |
| hexylquinolin-2- | (d, J = 7.5 Hz, 1H), 7.74(t, J = | 430.43 | R2 = 3-cyano- |
| yl](methyl)amino}butanoic | 8 Hz, 1H), 7.54 (d, J = 8.5 Hz, | Exact mass: 429.24 | Phenyl; R3 = |
| acid | 1H), 7.37 (d, J = 8.5 Hz, 1H), | | 2-methyl- |
| | 7.69 (dt, J = 7.6, 1.2 Hz, 1H), | | propionic acid; |
| | 7.17 (s, 1H), 6.98 (s, 1H), | | R4 = methyl |
| | 5.13 (s, 1H), 2.96 (s, 3H), | | |
| | 2.59-2.55(m, 2H), 2.4-23.3(m, | | |
| | 2H), 1.52-1.40(m, 2H), 1.30- | | |
| | 1.20(m, 6H), 1.18 (d, J = 6 Hz, | | |
| | 3H), 0.81 (t, J = 6.4 Hz, 3H) | | |
| 3-[(6-hexyl-4- | 400 MHz-DMSO-d6: 12.5 (s, | LCMS_5 min/2.90 | method A: R1 = |
| phenylquinolin-2- | 1H), 7.56-7.48 (m, 6H), 7.38 | Observed mass [M + H]: | n-hexyl; |
| yl)(methyl)amino]- | (d, J = 8.5 Hz, 1H), 7.30 (s, | 405.40 | R2 = Phenyl; |
| 2-methylpropanoic | 1H), 6.89 (s, 1H), 3.77(d, J = | Exact mass: 404.25 | R3 = 2-methyl- |
| acid | 8 Hz, 2H), 3.15 (s, 3H), 2.90- | | propionic acid; |
| | 2.80 (m, 1H), 2.57 (t, J = | | R4 = methyl |
| | 7.2 Hz, 2H), 1.52-1.40(m, | | |
| | 2H), 1.30-1.20(m, 6H), 1.07 | | |
| | (d, J = 6.8 Hz, 3H), 0.82 (t, | | |
| | J = 5.6 Hz, 3H) | | |
| 2-[methyl(6- | 400 MHz-DMSO-d6: 12.5 (s, | LCMS_LCMS_41/1.80 | method A: R1 = |
| pentanamido-4- | 1H), 9.90(s, 1H), 7.85-7.80 | Observed mass [M + H]: | n-pentyl |
| phenylquinolin-2- | (m, 2H), 7.65-7.45 (m, 4H), | 392.42 | amide; R2 = |
| yl)amino]acetic | 6.89 (s, 1H), 4.50 (m, 2H), | Exact mass: 391.19 | 3-cyano-Phenyl; |
| acid | 3.15 (s, 3H), 2.25 (m, 2H), | | R3 = ethyl acid; |
| | 1.52-1.40(m, 2H), 1.30- | | R4 = methyl |
| | 1.20(m,2H), 0.82 (t, J = | | |
| | 5.6 Hz, 3H) | | |
| 2-[methyl[6- | 400 MHz-DMSO-d6: 12.5 (s, | RND-FA-4.10 MIN/1.96 | method A: R1 = |
| (pentyloxy)-4- | 1H), 9.90(s, 1H), 7.58-7.52 | Observed mass [M + H]: | n-pentyl |
| phenylquinolin-2- | (m, 6H), 7.22(dd, J = 9.2, | 379.10 | ether; R2 = 3- |
| yl]amino}acetic | 2.8 Hz, 1H), 6.93-6.91 (m, | Exact mass: 378.19 | cyano-Phenyl; |
| acid | 2H), 4.38 (2, 2H), 3.849(t, J = | | R3 = ethyl acid; |
| | 6.8 Hz, 2H), 3.18 (s, 3H), | | R4 = methyl |
| | 2.25 (m, 2H), 1.69-1.65(m, | | |
| | 2H), 1.33-1.25(m, 4H), 0.86 | | |
| | (t, J = 5.6 Hz, 3H) | | |
| 2-[(7-bromo-4- | 400 MHz-DMSO-d6: 12.5 (s, | LCMS_5 min/2.73 | method A″: |
| phenylquinolin-2- | 1H), 7.73(s, 1H), 7.57-7.49 | Observed mass [M + H]: | R1 = bromo; ; |
| yl)(methyl)amino]acetic | (m, 6H), 7.27(dd, J = 8.8, | 371.45 | R2 = Phenyl; |
| acid | 2 Hz, 1H), 6.92(s, 1H), 4.28 | Exact mass: 370.03 | R3 = ethyl acid; |
| | (s, 2H), 3.19(s, 3H), | | R4 = methyl |
| 2-[(7-hexyl-4- | 400 MHz-DMSO-d6: 12.5 (s, | LCMS_5 min/2.86 | method A″: |
| phenylquinolin-2- | 1H), 7.57-7.49 (m, 5H), 7.45 | Observed mass [M + H]: | R1 = n-hexyl; ; |
| yl)(methyl)amino]acetic | (d, J = 8.8 Hz, 1H), 7.40(s, | 377.40 | R2 = Phenyl; |
| acid | 1H), 7.03(dd, J = 8.8, 1.6 Hz, | Exact mass: 376.22 | R3 = ethyl acid; |
| | 1H), 6.87(s, 1H), 4.42 (s, 2H), | | R4 = methyl |
| | 3.18 (s, 3H), 2.68(t, J = | | |
| | 7.2 Hz, 2H), 1.65-1.59(m, | | |
| | 2H), 1.35-1.20(m, 6H), 0.86 | | |
| | (t, J = 7.2 Hz, 3H) | | |
| 2-[methyl(6-octyl- | 400 MHz-DMSO-d6: 12.5 (s, | RND-FA-3.5/2.515 | method A: R1 = |
| 4-phenylquinolin-2- | 1H), 7.58-7.49 (m, 6H), 7.41- | Observed mass [M + H]: | n-octyl; ; |
| yl)amino]acetic | 7.36 (m, 3H), 7.39(dd, J = 8.8, | 405.10 | R2 = Phenyl; |
| acid | 1.6 Hz, 1H), 6.91(s, 1H), 4.42 | Exact mass: 404.25 | R3 = ethyl acid; |
| | (s, 2H), 3.19 (s, 3H), 2.57- | | R4 = methyl |
| | 2.54 (m, 2H), 1.55-1.50(m, | | |
| | 2H), 1.30-1.20(m, 10H), | | |
| | 0.83 (t, J = 7.2 Hz, 3H) | | |
| 3-{[6-hexyl-4- | 400 MHz-DMSO-d6: 12.5 (s, | RND-FA-3.5 min/1.772 | method A: R1 = |
| (pyridin-3- | 1H), 8.71(m, 2H), 7.95(d, J = | Observed mass [M + H]: | n-hexyl; |
| yl)quinolin-2- | 8 Hz, 1H), 7.60-7.54(m, 2H), | 406.10 | R2 = 3-pyridyl; |
| yl](methyl)amino}- | 7.39(d, J = 8.4 Hz, 1H), 7.20 | Exact mass: 405.24 | R3 = 2-methyl- |
| 2-methylpropanoic | (s, 1H), 6.97(s, 1H), 3.75(s, | | propionic acid; |
| acid | 2H), 3.17 (s, 3H), 2.58 (t, J = | | R4 = methyl |
| | 7.6 Hz, 2H), 1.55-1.50(m, | | |
| | 2H), 1.30-1.20(m, 6H), | | |
| | 1.04(d, J = 7.2 Hz, 3H), 0.82 | | |
| | (t, J = 6.8 Hz, 3H) | | |
| 2-{[6-hexyl-4- | 400 MHz-DMSO-d6: 12.5 (s, | LCMS_5 min/2.64 | method A: R1 = |
| (pyridin-3- | 1H), 8.70-8.67(m, 2H), | Observed mass [M + H]: | n-hexyl; |
| yl)quinolin-2- | 7.91(d, J = 8 Hz, 1H), 7.57(dd, | 378.42 | R2 = 3-pyridyl; |
| yl](methyl)amino}acetic | J = 7.6, 4.8 Hz, 1H), 7.51(d, J = | Exact mass: 377.21 | R3 = ethyl acid; |
| acid | 8.4 Hz, 1H), 7.36(m, 1H), | | R4 = methyl |
| | 7.20 (s, 1H), 6.79(s, 1H), 4.00 | | |
| | (s, 2H), 3.17 (s, 3H), 2.57 (t, | | |
| | J = 7.6 Hz, 2H), 1.55-1.50(m, | | |
| | 2H), 1.30-1.20(m, 6H), 0.82 | | |
| | (t, J = 6.8 Hz, 3H) | | |
| 2-{[4-(3- | 400 MHz-DMSO-d6: 13.2 (s, | LCMS_5 min/3.04 | method B: R1 = |
| cyanophenyl)-6- | 1H), 8.02-7.99 (m, 2H), 7.83 | Observed mass [M + H]: | n-hexyl; |
| hexylquinolin-2- | (d, J = 8.0 Hz, 1H), 7.76(t, J = | 389.31 | R2 = 3-cyano- |
| yl]oxy]acetic acid | 8 Hz, 1H), 7.52 (dd, J = 8.5, | Exact mass: 388.18 | phenyl; R3 = |
| | 1.6 Hz, 1H), 7.42 (d, J = | | ethyl acid |
| | 8.5 Hz, 1H), 7.12 (s, 1H), 6.60 | | |
| | (s, 1H), 5.05(s, 2H), 2.56 (t, J = | | |
| | 7.2 Hz, 2H), 1.52-1.40(m, | | |
| | 2H), 1.23 (m, 6H), 0.82 (t, J = | | |
| | 6.4 Hz, 3H) | | |
| 3-{[4-(3- | 400 MHz-DMSO-d6: 12.2 (s, | LCMS_5 min/2.80 | method A: R1 = |
| cyanophenyl)-6- | 1H), 8.01-7.97 (m, 2H), 7.84 | Observed mass [M + H]: | n-hexyl; |
| hexylquinolin-2- | (d, J = 8.0 Hz, 1H), 7.76 (t, J = | 430.43 | R2 = 3-cyano- |
| yl](methyl)amino}- | 8 Hz, 1H), 7.57 (d, J = 8.4 Hz, | Exact mass: 429.24 | phenyl; R3 = 2-methyl |
| 2-methylpropanoic | 1H), 7.41 (d, J = 8.5 Hz, 1H), | | propionic acid; |
| acid | 7.19 (s, 1H), 6.98 (s, 1H), | | R4 = methyl |
| | 3.81(d, J = 7.2 Hz, 2H), | | |
| | 3.16(s, 3H), 2.94-2.89(m, | | |
| | 1H), 2.56 (t, J = 7.2 Hz, 2H), | | |
| | 1.52-1.40(m, 2H), 1.23 (m, | | |
| | 6H), 1.10(d, J = 7.2 Hz, 3H), | | |
| | 0.82 (t, J = 6.4 Hz, 3H) | | |
| 2-{[4-(3- | 400 MHz-DMSO-d6: 12.5(s, | RND X-bridge 5.0 min/ | method A: R1 = |
| cyanophenyl)-6- | 1H), 7.98-7.96(m, 2H), | 2.385 | n-hexyl; |
| hexylquinolin-2- | 7.80(d, J = 7.6 Hz, 1H), | Observed mass: | R2 = 3-cyano- |
| yl](methyl)amino}acetic | 7.74(t, J = 8.4 Hz, 1H), 7.50(d, | Exact mass: 401.21 | phenyl; |
| acid | J = 8.8 Hz, 1H), 7.36(dd, J = | | R3 = ethyl acid; |
| | 8.8, 1.6 Hz, 1H), 7.16 (s, 1H), | | R4 = methyl |
| | 6.80 (s, 1H), 4.01 (s, 2H), | | |
| | 3.16 (s, 3H), 2.57 (t, J = 8 Hz, | | |
| | 2H), 1.55-1.47 (m, 2H), 1.36- | | |
| | 1.22(m, 6H), 0.82 (t, J = | | |
| | 6.5 Hz, 3H) | | |
| 1-[6-hexyl-4- | 400 MHz-DMSO-d6: 12.5(s, | LCMS_5 MIN/2.66 | method A: R1 = |
| (pyridin-3- | 1H), 8.72-8.70(m, 2H), 7.95 | Observed mass [M + H]: | n-hexyl; |
| yl)quinolin-2- | (d, J = 7.6 Hz, 1H), 7.62- | 418.43 | R2 = 3-pyridyl; |
| yl]piperidine-3- | 7.58(m, 2H), 7.42(d, J = | Exact mass: 417.24 | R3-R4 = 2- |
| carboxylic acid | 8.4 Hz, 1H), 7.23(s, 1H), | | carboxylic |
| | 7.18(s, 1H), 4.55(d, J = | | acid piperidyl |
| | 12.8 Hz, 1H), 4.27(d, J = | | |
| | 12.8 Hz, 1H), 3.21-3.10 (m, | | |
| | 2H), 2.59 (t, J = 8 Hz, 2H), | | |
| | 2.07-1.97(m, 1H), 1.75-1.64 | | |
| | (m, 2H), 1.54-1.48 (m, 2H), | | |
| | 1.32-1.26(m, 6H), 0.82 (t, J = | | |
| | 6.5 Hz, 3H) | | |
| 1-(6-hexyl-4- | 400 MHz-DMSO-d6: 12.5(s, | RND-FA-3.5 min/2.049 | method A: R1 = |
| phenylquinolin-2- | 1H), 7.59-7.49(m, 6H), 7.40 | Observed mass [M + H]: | n-hexyl; R2 = |
| yl)piperidine-3- | (dd, J = 8.4, 1.6 Hz, 1H), | 417.10 | phenyl; R3-R4 = |
| carboxylic acid | 7.30(s, 1H), 7.06(s, 1H), | Exact mass: 416.25 | 2-carboxylic |
| | 4.52(d, J = 12.8 Hz, 1H), | | acid piperidyl |
| | 4.23(d, J = 12.8 Hz, 1H), | | |
| | 3.31-3.11 (m, 2H), 2.59 (t, J = | | |
| | 8 Hz, 2H), 2.07-1.97(m, 1H), | | |
| | 1.75-1.64 (m, 2H), 1.54-1.48 | | |
| | (m, 2H), 1.32-1.26(m, 6H), | | |
| | 0.82 (t, J = 6.5 Hz, 3H) | | |
| 2-{[6-butyl-4-(4- | 400 MHz-DMSO-d6: 12.5(s, | RND-FA-3.5 min/1.823 | method A: R1 = |
| hydroxyphenyl)quinolin-2- | 1H), 9.72 (s, 1H), 7.52 (d, J = | Observed mass [M + H]: | n-butyl; R2 = 4- |
| yl](methyl)amino}acetic | 8.4 Hz, 1H), 7.40-7.32(m, | 365.00 | hydroxyl-phenyl; |
| acid | 4H), 6.94 (d, J = 8.4 Hz, 1H), | Exact mass: 364.18 | R3 = ethyl acid; |
| | 6.84(s, 1H) 4.41 (s, 2H), 3.18 | | R4 = methyl |
| | (s, 3H), 2.60 (t, J = 8 Hz, 2H), | | |
| | 1.54-1.48 (m, 2H), 1.32- | | |
| | 1.26(m, 2H), 0.87 (t, J = | | |
| | 6.5 Hz, 3H) | | |
| 2-{[6-butyl-4-(3- | 400 MHz-DMSO-d6: 12.5(s, | LCMS_5 min/2.59 | method A: R1 = |
| hydroxyphenyl)quinolin-2- | 1H), 9.65 (s, 1H), 7.52 (d, J = | Observed mass [M + H]: | n-butyl; R2 = |
| yl](methyl)amino}acetic | 8.0 Hz, 1H), 7.40-7.32(m, | 365.31 | 3-hydroxyl-phenyl; |
| acid | 3H), 7.30 (d, J = 8.0 Hz, 1H), | Exact mass: 364.18 | R3 = ethyl acid; |
| | 6.90-6.87(m, 4H) 4.41 (s, | | R4 = methyl |
| | 2H), 3.18 (s, 3H), 2.59 (t, J = | | |
| | 8 Hz, 2H), 1.54-1.48 (m, 2H), | | |
| | 1.32-1.26(m, 2H), 0.86 (t, J = | | |
| | 6.5 Hz, 3H) | | |
| 2-{[6-butyl-4-(2- | 400 MHz-DMSO-d6: 12.5(s, | LCMS_5 min/2.59 | method A: R1 = |
| hydroxyphenyl)quinolin-2- | 1H), 9.46 (s, 1H), 7.50 (d, J = | Observed mass [M + H]: | n-butyl; |
| yl](methyl)amino}acetic | 8.4 Hz, 1H), 7.36-7.28(m, | 365.37 | R2 = 2-hydroxyl- |
| acid | 2H), 7.30 (d, J = 8.0 Hz, 1H), | Exact mass: 364.18 | phenyl; |
| | 7.16(d, J = 6.4 Hz, 1H), | | R3 = ethyl acid; |
| | 7.09(s, 1H), 7.00(d, J = 6.8 | | R4 = methyl |
| | Hz, 1H), 6.94(t, J = 7.2 Hz, 1H), | | |
| | 6.84(s, 1H), 4.39(d, | | |
| | J = 4.4 Hz, 2H), 3.16 (s,3H), | | |
| | 2.54 (t, J = 8 Hz, 2H), 1.54- | | |
| | 1.48 (m, 2H), 1.31-1.26(m, | | |
| | 2H), 0.85 (t, J = 6.5 Hz, 3H) | | |
| 2-{[6-butyl-4-(4- | 400 MHz-DMSO-d6: 12.5(s, | RND-FA-10/2.218 | method A: R1 = |
| fluorophenyl)quinolin-2- | 1H), 7.55-7.52(m, 3H), 7.41- | Observed mass [M + H]: | n-butyl; |
| yl](methyl)amino}acetic | 7.37(m, 3H), 7.27 (s, 1H), | 367.00 | R2 = 4-fluoro- |
| acid | 6.84(s, 1H), 4.25 (s, 2H), | Exact mass: 366.17 | phenyl; |
| | 3.18 (s, 3H), 2.58 (t, J = 8 Hz, | | R3 = ethyl acid; |
| | 2H), 1.55-1.49 (m, 2H), 1.32- | | R4 = methyl |
| | 1.26(m, 2H), 0.86 (t, J = | | |
| | 6.5 Hz, 3H) | | |
| 2-{[6-butyl-4-(3- | | Observed mass: | |
| fluorophenyl)quinolin-2- | | Exact mass: 366.17 | |
| yl](methyl)amino}acetic | | | |
| acid | | | |
| 2-{[6-butyl-4-(2- | | Observed mass: | |
| fluorophenyl)quinolin-2- | | Exact mass: 366.17 | |
| yl](methyl)amino}acetic | | | |
| acid | | | |
| 2-{[6-butyl-4-(4- | 400 MHz-DMSO-d6: 12.5(s, | RND-FA-10min/ 2.279 | method A: R1 = |
| methylphenyl)quinolin-2- | 1H), 7.49(d, J = 8.4 Hz, 1H), | Observed mass [M + H]: | n-butyl; |
| yl](methyl)amino}acetic | 7.37-7.30(m, 6H), 6.69(s, | 363.00 | R2 = 4-methyl- |
| acid | 1H), 3.93 (s, 2H), 3.16 (s, | Exact mass: 362.20 | phenyl; |
| | 3H), 2.56 (t, J = 8 Hz, 2H), | | R3 = ethyl acid; |
| | 2.40(s, 3H), 1.53-1.47 (m, | | R4 = methyl |
| | 2H), 1.32-1.26(m, 2H), 0.86 | | |
| | (t, J = 7.2 Hz, 3H) | | |
| 2-{[6-butyl-4-(3- | | Observed mass: | |
| methylphenyl)quinolin- | | Exact mass: 362.20 | |
| 2-yl](methyl)amino}acetic | | | |
| acid | | | |
| 2-{[6-butyl-4-(2- | 500 MHz-DMSO-d6: 12.5(s, | LCMS_5 MIN/2.95 | method A: R1 = |
| methylphenyl)quinolin-2- | 1H), 7.54(d, J = 8.5 Hz, 1H), | Observed mass [M + H]: | n-butyl; |
| yl](methyl)amino}acetic | 7.40-7.34(m, 4H), 7.21(d, J = | 363.42 | R2 = 2-methyl- |
| acid | 6.5 Hz, 1H), 6.86 (s, 1H), | Exact mass: 362.20 | phenyl; |
| | 6.85 (s, 1H), 4.44(d, 1H), 4.37 | | R3 = ethyl acid; |
| | (d, 1H), 3.16 (s, 3H), 2.60 (m, | | R4 = methyl |
| | 2H), 2.00 (s, 3H), 1.53-1.47 | | |
| | (m, 2H), 1.26-1.16(m, 2H), | | |
| | 0.83 (t, J = 7.2 Hz, 3H) | | |
| 2-{[6-butyl-4-(4- | 500 MHz-DMSO-d6: 12.5(s, | LCMS_5 MIN/2.74 | method A: R1 = |
| cyanophenyl)quinolin-2- | 1H), 8.04(d, J = 6.5 Hz, 2H), | Observed mass [M + H]: | n-butyl; |
| yl](methyl)amino}acetic | 7.72(d, J = 6.5 Hz, 2H), 7.56 | 374.30 | R2 = 4-cyano- |
| acid | (d, J = 8.5 Hz, 1H), 7.43 (d, | Exact mass: 373.18 | phenyl; |
| | J = 8.5 Hz, 1H), 7.22(s, 1H), | | R3 = ethyl acid; |
| | 6.97 (s, 1H), 4.42(s, 2H), | | R4 = methyl |
| | 3.19 (s, 3H), 2.60 (m, 2H), | | |
| | 1.53-1.47 (m, 2H), 1.26- | | |
| | 1.16(m, 2H), 0.86 (t, J = | | |
| | 7.2 Hz, 3H) | | |
| 2-{[6-butyl-4-(4- | 500 MHz-DMSO-d6: 12.5(s, | RND-FA-3.5/1.657 | method A: R1 = |
| carbamoylphenyl)quinolin-2- | 1H), 8.10(s, 1H), 8.04(d, J = | Observed mass [M + H]: | n-butyl; |
| yl](methyl)amino}acetic | 8.4 Hz, 2H), 8.04(d, J = 6.4 | 392.00 | R2 = 4-amide- |
| acid | Hz, 2H), 7.58-7.71(m, 3H), | Exact mass: 391.19 | phenyl; |
| | 7.48(s, 1H), 7.38 (d, J = 8.4 | | R3 = ethyl acid; |
| | Hz, 1H), 7.27(s, 1H), 6.84 (s, | | R4 = methyl |
| | 1H), 4.19 (s,2H), 3.18 (s, | | |
| | 3H), 2.57 (t, J = 7.6 Hz, 2H), | | |
| | 1.53-1.47 (m, 2H), 1.26- | | |
| | 1.16(m, 2H), 0.86 (t, J = | | |
| | 7.2 Hz, 3H) | | |
| 2-{[6-butyl-4- | 500 MHz-DMSO-d6: 12.5(s, | ANL_MCL5/6.317 | method A: R1 = |
| (pyridin-4- | 1H), 8.70(d, J = 4.5 Hz, 2H), | Observed mass [M + H]: | n-butyl; |
| yl)quinolin-2- | 7.65(d, J = 9 Hz, 1H), 7.57 (d, | 350.31 | R2 = 4-pyridyl; |
| yl](methyl)amino}acetic | J = 4.5 Hz, 2H), 7.41(d, J = | Exact mass: 349.18 | R3 = ethyl acid; |
| acid | 9 Hz, 1H), 7.25 (s, 1H), | | R4 = methyl |
| | 6.88(s, 1H), 4.27 (s, 2H), | | |
| | 3.30 (s, 3H), 2.62 (t, J = 8 Hz, | | |
| | 2H), 1.58-1.51 (m, 2H), 1.35- | | |
| | 1.26(m, 2H), 0.90 (t, J = | | |
| | 6.5 Hz, 3H) | | |
| 6-butyl-2- | 400 MHz-DMSO-d6: 13.2(s, | LCMS_5_MIN/2.85 | |
| (carboxymethoxy)- | 1H), 7.58-7.51 (m, 4H), | Observed mass [M + H]: | |
| 4-phenylquinoline- | 7.44(d, J = 8.8 Hz, 1H), 7.37- | 380.22 | |
| 3-carboxylic acid | 7.35(m, 2H), 6.94 (s, 1H), 5.02 | Exact mass: 379.14 | |
| | (s, 2H), 2.52 (t, J = 7.6 Hz, | | |
| | 2H), 1.55-1.51(m, 2H), 1.30- | | |
| | 1.26(m, 2H), 0.82 (t, J = 8 Hz, | | |
| | 3H) | | |
| 2-{[6-butyl-4- | 500 MHz-DMSO-d6: 12.5(s, | LCMS_5 MIN/2.49 | method A: R1 = |
| (pyridin-3- | 1H), 8.67(m, 2H), 8.00 (d, J = | Observed mass [M + H]: | n-butyl; |
| yl)quinolin-2- | 7.6 Hz, 1H), 7.69 (d, J = | 350.40 | R2 = 3-pyridyl; |
| yl](methyl)amino}acetic | 8.5 Hz, 1H), 7.64-7.61 (m, | Exact mass: 349.18 | R3 = ethyl acid; |
| acid | 1H), 7.43(d, J = 8.5 Hz, 1H), | | R4 = methyl |
| | 7.24(s, 1H), 6.90(s, 1H), | | |
| | 4.28(s, 2H), 3.30 (s, 3H), 2.59 | | |
| | (t, J = 8 Hz, 2H), 2.07-1.97(s, | | |
| | 1H), 1.75-1.64 (m, 2H), 1.54- | | |
| | 1.48 (m, 2H), 0.82 (t, J = | | |
| | 6.5 Hz, 3H) | | |
| 1-[6-butyl-4-(3- | 400 MHz-DMSO-d6: 12.5(s, | RND-FA-3.5/1.986 | method A: R1 = |
| cyanophenyl)quinolin- | 1H), 8.05-7.95(m, 2H), 7.85 | Observed mass [M + H]: | n-hexyl; |
| 2-yl]piperidine- | (d, J = 7.6 Hz, 1H), 7.76(t, | 414.00 | R2 = 3-cyano- |
| 3-carboxylic acid | J = 7,6 Hz, 1), 7.69 (d, J = | Exact mass: 413.21 | phenyl; R3- |
| | 8.8 Hz, 1H), 7.69 (dd, J = 8.4, | | R4 = 3- |
| | 1.6 Hz, 1H), 7.21(s, 1H), | | carboxylic |
| | 7.17(s, 1H), 4.55(d, J = | | acid piperidyl |
| | 12.8 Hz, 1H), 4.27(d, J = | | |
| | 12.8 Hz, 1H), 3.21-3.10 (m, | | |
| | 2H), 2.59 (t, J = 8 Hz, 2H), | | |
| | 2.07-1.97(m, 1H), 1.75-1.64 | | |
| | (m, 2H), 1.54-1.48 (m, 2H), | | |
| | 1.32-1.26(m, 2H), 0.86 (t, J = | | |
| | 6.5 Hz, 3H) | | |
| 4-(6-butyl-4- | 400 MHz-DMSO-d6: 12.4(br | LCMS_5 min/2.84 | method A: R1 = |
| phenylquinolin-2- | s, 1H), 7.61(d, J = 8.0 Hz, | Observed mass [M + H]: | n-hexyl; |
| yl)morpholine-2- | 1H), 7.58-7.45(m, 5H), | 391.37 | R2 = phenyl; |
| carboxylic acid | 7.41(d, J = 7.6 Hz, 1H), | Exact mass: 390.19 | R3-R4 = 3- |
| | 7.32(s, 1H), 7.01(s, 1H), | | carboxylic acid |
| | 4.53(m, 1H), 4.23(m, 2H), | | morpholino |
| | 3.78(m, 1H), 3.52(m, 1H), | | |
| | 3.06(m, 1H), 2.94(m, 1H), | | |
| | 2.60-2.56 (m, 2H), 1.53-1.47 | | |
| | (m, 2H), 1.32-1.26(m, 2H), | | |
| | 0.86 (t, J = 7.2 Hz, 3H) | | |
| 1-(6-butyl-4- | 400 MHz-DMSO-d6: 12.4(br s, | RND-FA-3.5 min/2.051 | method A: R1 = |
| phenylquinolin-2- | 1H), 7.85(d, J = 8.0 Hz, | Observed mass [M + H]: | n-hexyl; |
| yl)piperidine-3- | 1H), 7.70-7.45(m, 5H), | 389.10 | R2 = phenyl; |
| carboxylic acid | 7.41(d, J = 7.6 Hz, 1H), | Exact mass: 388.22 | R3-R4 = 3- |
| | 7.31(s, 1H), 7.07(s, 1H), | | carboxylic |
| | 4.53(d, J = 13.2 Hz, 1H), | | acid piperidyl |
| | 4.23(d, J = 13.2 Hz, 1H), 3.22- | | |
| | 3.10 (m,2H), 2.60-2.56 (m, | | |
| | 2H), 2.05-1.96(m, 1H), 1.80- | | |
| | 1.60(m, 2H), 1.53-1.47 (m, | | |
| | 2H), 1.32-1.26(m, 3H), 0.86 | | |
| | (t, J = 7.2 Hz, 3H) | | |
| 3-{[6-butyl-4-(3- | 400 MHz-DMSO-d6: 12.4(br | LCMS_5 min/2.73 | method A: R1 = |
| cyanophenyl)quinolin-2- | s, 1H), 8.00-7.97(m, 2H), | Observed mass [M + H]: | n-butyl; |
| yl](methyl)amino}- | 7.85(d, J = 8.0 Hz, 1H), 7.76(t, | 402.30 | R2 = 3-cyano- |
| 2-methylpropanoic | J = 8 Hz, 1H), 7.56(d, J = | Exact mass: 401.21 | phenyl; R3 = 2- |
| acid | 8.8 Hz, 2H), 7.41(dd, J = 8.4, | | methyl propionic |
| | 2 Hz, 1H), 7.19(d, J = 1.6 Hz, | | acid; |
| | 1H), 6.98(s, 1H), 3.78 (d, | | R4 = methyl |
| | J = 6.8 Hz, 2H), 3.16 (s, 3H), | | |
| | 2.95-2.82 (m, 1H), 2.60-2.54 | | |
| | (m, 2H), 1.53-1.47 (m, 2H), | | |
| | 1.32-1.26(m, 2H), 1.08 (d, J = | | |
| | 7.2 Hz, 3H), 0.86 (t, J = 7.2 Hz, | | |
| | 3H) | | |
| 3-{[6-butyl-4- | 400 MHz-DMSO-d6: 8.70(s, | LCMS_5 min/2.54 | method A: R1 = |
| (pyridin-3- | 2H), 7.95(d, J = 7.6 Hz, 1H), | Observed mass [M + H]: | n-butyl; |
| yl)quinolin-2- | 7.60-7.53(m, 2H), 7.38(d, J = | 378.31 | R2 = 3-pyridyl; |
| yl](methyl)amino}- | 8.4 Hz, 1H), 7.20(s, 1H), | Exact mass: 377.21 | R3 = 2-methyl |
| 2-methylpropanoic | 6.97(s, 1H), 3.73 (s, 2H), | | propionic acid; |
| acid | 3.16 (s, 3H), 2.59-2.54 (m, | | R4 = methyl |
| | 3H), 1.53-1.47 (m, 2H), 1.32- | | |
| | 1.26(m, 2H), 1.00 (d, J = | | |
| | 6.4 Hz, 3H), 0.86 (t, J = 7.2 Hz, | | |
| | 3H) | | |
| 3-[(6-butyl-4- | 400 MHz-DMSO-d6-D2O | RND-FA-3.5 min/2.085 | method A: R1 = |
| phenylquinolin-2- | exchange: 7.80(br s, 1H), | Observed mass [M + H]: | n-butyl; |
| yl)(methyl)amino]- | 7.64-7.43(m, 6H), 7.37(s, | 377.10 | R2 = phenyl; |
| 2-methylpropanoic | 1H), 7.15 (br s, 1H), 4.00(s, | Exact mass: 376.22 | R3 = 2-m ethyl |
| acid | 1H), 3.83-3.78 (m, 1H), 3.28 | | propionic acid; |
| | (s, 3H), 2.94(dd, J = 14.8, | | R4 = methyl |
| | 7.8 Hz, 1H), 2.62(t, J = 8 Hz, | | |
| | 2H), 1.53-1.47 (m, 2H), 1.36- | | |
| | 1.19(m, 3H), 1.13 (d, J = | | |
| | 6.4 Hz, 3H), 0.86 (t, J = 7.2 Hz, | | |
| | 3H) | | |
| 3-[(6-butyl-4- | 400 MHz-DMSO-d6: 7.64- | ANL-MCL3/2.03 | method A: R1 = |
| phenylquinolin-2- | 7.43(m, 6H), 7.38(d, J = | Observed mass [M + H]: | n-butyl; |
| yl)(methyl)amino]butanoic | 8.4 Hz, 1H), 7.37(s, 1H), | 377.24 | R2 = phenyl; |
| acid | 7.29(s, 1H), 6.91 (br s, 1H), | Exact mass: 376.22 | R3 = l-methyl |
| | 5.20 (s, 1H), 2.96 (s, 3H), | | propionic acid; |
| | 2.94(dd, J = 14.8, 7.8 Hz, 1H), | | R4 = methyl |
| | 2.57(t, J = 8 Hz, 2H), 1.53- | | |
| | 1.47 (m, 2H), 1.32-1.22(m, | | |
| | 3H), 1.20 (d, J = 6.8 Hz, 3H), | | |
| | 0.86 (t, J = 7.2 Hz, 3H) | | |
| 3-[(6-butyl-4- | 400 MHz-DMSO-d6: 7.64- | RND-FA-3.5 min/1.992 | method A: R1 = |
| phenylquinolin-2- | 7.43(m, 6H), 7.37(d, J = | Observed mass [M + H]: | n-butyl; |
| yl)(methyl)amino]propanoic | 8.8 Hz, 1H), 7.28(s, 1H), 6.86 | 363.10 | R2 = phenyl; |
| acid | (br s, 1H), 5.20 (s, 1H), 3.8(s, | Exact mass: 362.20 | R3 = n- |
| | 2H), 3.14 (s, 3H), 2.57(t, J = | | propionic |
| | 8 Hz, 2H), 2.328(s, 2H), 1.53- | | acid; |
| | 1.47 (m, 2H), 1.32-1.22(m, | | R4 = methyl |
| | 3H), 0.85 (t, J = 7.2 Hz, 3H) | | |
| N-(6-butyl-4-(3- | 400 MHz-DMSO-d6: 11.8(s, | LCMS_5 MIN/3.07 | method A: R1 = |
| cyanophenyl)quinolin- | 1H), 8.01-7.97(m, 2H), 7.84- | Observed mass [M + H]: | n-butyl; |
| 2-yl)-N- | 7.81(m, 1H), 7.76(t, J = | 416.28 | R2 = 3-cyano- |
| methylvaline? | 8.4 Hz, 1H), 7.51(d, J = 7.2 Hz, | Exact mass: 415.23 | phenyl; R3 = 1- |
| | 1H), 7.43-7.35 (m1H), 7.15 | | isopropyl- |
| | (s, 1H), 7.05 (s, 1H), 4.42 (s, | | ethyl acid; |
| | 2H), 3.11 (s, 3H), 2.54 (t, J = | | R4 = methyl |
| | 8 Hz, 2H), 2.33-2.19(m, 1H), | | |
| | 1.55-1.47 (m, 2H), 1.32- | | |
| | 1.22(m, 2H), 1.01 (d, J = | | |
| | 6.4 Hz, 3H), 0.87(t, J = 6.4 Hz, | | |
| | 3Hh), 0.78 (d, J = 6.4 Hz, 3H) | | |
| 2-{[4-(3- | 400 MHz-DMSO-d6: 8.00(dd, | ANL_MCL5/6.615 | method B: R1 = |
| cyanophenyl)-6- | J = 7.6, 1.6 Hz, 1H), 7.96(s, | Observed mass [M + H]: | n-pentyl; |
| pentylquinolin-2- | 1H), 7.82-7.74 (m, 2H), | 375.23 | R2 = 3-cyano- |
| yl]oxy}acetic acid | 7.42(d, J = 8.0 Hz, 1H), | Exact mass: 374.16 | phenyl; R3 = |
| | 7.28(d, J = 8.0 Hz, 1H), | | ethyl acid |
| | 7.07(d, J = 1.2 Hz, 1H), 6.53 | | |
| | (s, 1H), 4.65 (s, 2H), 2.54(t, J = | | |
| | 8 Hz, 2H), 1.50-1.45 (m, | | |
| | 2H), 1.27-1.19(m, 4H), 0.82 | | |
| | (t, J = 7.2 Hz, 3H) | | |
| 2-{[4-(3- | 400 MHz-DMSO-d6: 13.2 (s, | RND-FA-3.5/1.996 | method B: R1 = |
| carbamoylphenyl)- | 1H), 8.08(s, 1H), 8.04- | Observed mass [M + H]: | n-pentyl; |
| 6-pentylquinolin-2- | 8.01(m, 1H), 7.99(s, 1H), | 393.00 | R2 = 3-amido- |
| yl]oxy]acetic acid | 7.67-7.64 (m, 2H), 7.51- | Exact mass: 392.17 | phenyl; R3 = |
| | 7.41(m, 3H), 7.20(d, J = | | ethyl acid |
| | 1.2 Hz, 1H), 6.59 (s, 1H), 5.07 | | |
| | (s, 2H), 2.55(t, J = 8 Hz, 2H), | | |
| | 1.53-1.45 (m, 2H), 1.28- | | |
| | 1.19(m, 4H), 0.82 (t, J = | | |
| | 7.2 Hz, 3H) | | |
| 2-{[4-(3- | 400 MHz-DMSO-d6: 8.01- | RND-FA-3.5/2.142 | method B: R1 = |
| cyanophenyl)-6- | 7.97(m, 2H), 7.83-7.74 (m, | Observed mass [M + H]: | n-propyl; |
| propylquinolin-2- | 2H), 7.44(d, J = 8.0 Hz, 1H), | 347.00 | R2 = 3-cyano- |
| yl]oxy]acetic acid | 7.31(d, J = 8.0 Hz, 1H), | Exact mass: 346.13 | phenyl; R3 = |
| | 7.08(d, J = 1.2 Hz, 1H), 6.55 | | ethyl acid |
| | (s, 1H), 4.74 (s, 2H), 2.52(t, J = | | |
| | 8 Hz, 2H), 1.50-1.45 (m, | | |
| | 2H), 0.84 (t, J = 7.2 Hz, 3H) | | |
| 2-{[4-(3- | 400 MHz-DMSO-d6: 13.2 (s, | RND-FA-3.5/1.826 | method B: R1 = |
| carbamoylphenyl)- | 1H), 8.08(s, 1H), 8.04- | Observed mass [M + H]: | n-propyl; |
| 6-propylquinolin-2- | 8.01(m, 1H), 7.99(s, 1H), | 365.00 | R2 = 3-amido- |
| yl]oxy]acetic acid | 7.67-7.64 (m, 2H), 7.51- | Exact mass: 364.14 | phenyl; R3 = |
| | 7.40(m, 3H), 7.20(d, J = | | ethyl acid |
| | 1.6 Hz, 1H), 6.59 (s, 1H), 5.03 | | |
| | (s, 2H), 2.55(t, J = 8 Hz, 2H), | | |
| | 1.53-1.45 (m, 2H), 0.84 (t, J = | | |
| | 7.2 Hz, 3H) | | |
| 2-{[4-(3- | 400 MHz-DMSO-d6: 13.2 (s, | RND-FA-6.0/1.128 | method B: R1 = |
| carbamoylphenyl)- | 1H), 8.09(s, 1H), 8.04- | Observed mass [M + H]: | ethyl; R2 = |
| 6-ethylquinolin-2- | 8.02(m, 1H), 7.99(s, 1H), | 351.00 | 3-amido-phenyl; |
| yl]oxy]acetic acid | 7.66-7.64 (m, 2H), 7.52 (d, J = | Exact mass: 350.13 | R3 = ethyl acid |
| | 7.6 Hz, 1H), 7.46-7.42(m, | | |
| | 2H), 7.22(s 1H), 6.59 (s, 1H), | | |
| | 5.06 (s, 2H), 2.55(q, J = | | |
| | 7.6 Hz, 2H), 1.11(t, J = 7.2 Hz, | | |
| | 3H) | | |
| 2-{[6-bromo-4-(3- | 400 MHz-DMSO-d6: 13.2(s, | RND-FA-3.5/1.975 | method B: R1 = |
| cyanophenyl)quinolin- | 1H), 8.05-8.02(m, 2H), 7.87- | Observed mass [M + H]: | bromo; |
| 2-yl]oxy}acetic | 7.76 (m, 3H), 7.51(d, J = | 382.90 | R2 = 3-cyano- |
| acid | 8.8 Hz, 1H), 7.38 (d, J = | Exact mass: 382.00 | phenyl; R3 = |
| | 1.2 Hz, 1H), 6.70 (s, 1H), 5.07 | | ethyl acid |
| | (s, 2H) | | |
| 2-{[6-bromo-4-(3- | 400 MHz-DMSO-d6: 13.2 (s, | RND-FA-3.5/1.652 | method B: R1 = |
| carbamoylphenyl)quinolin-2- | 1H), 8.10(s, 1H), 8.06- | Observed mass [M + H]: | bromo; |
| yl]oxy]acetic acid | 8.02(m, 1H), 7.99(s, 1H), | 400.90 | R2 = 3-amido- |
| | 7.80 (dd, J = 8.8, 2 Hz, 1H), | Exact mass: 400.01 | phenyl; R3 = |
| | 7.80 (d, J = 5.2 Hz, 2H), 7.51(d, | | ethyl acid |
| | J = 8.8 Hz, 1H), 7.48(s, 1H), | | |
| | 7.44(d, J = 2 Hz, 1H), 6.69 (s, | | |
| | 1H), 5.07(s, 2H) | | |
| 2-{[6-butyl-4-(3- | 400 MHz-DMSO-d6: 12.5(s, | RND-FA-3.5 min/2.471 | method B: R1 = |
| cyanophenyl)quinol | 1H), 8.03-8.01(m, 2H), 7.84 | Observed mass [M + H]: | n-butyl; |
| in-2-yl]oxy}acetic | (m, 1H), 7.79 (t, J = 7.6 Hz, | 361.00 | R2 = 3-cyano- |
| acid | 1H), 7.73(d, J = 8.4 Hz, 1H), | Exact mass: 360.15 | phenyl; R3 = |
| | 7.58(dd, J = 8.8, 2.0 Hz, 1H), | | ethyl acid |
| | 7.37 (s, 1H), 7.06 (s, 1H), 4.97 | | |
| | (s, 2H), 2.66 (t, J = 7.6 Hz, | | |
| | 2H), 1.55-1.51(m, 2H), 1.30- | | |
| | 1.26(m, 2H), 0.87 (t, J = 8 Hz, | | |
| | 3H) | | |
| 2-{[6-butyl-4-(3- | 400 MHz-DMSO-d6: 13.2 (s, | RND-FA-3.5 min/2.179 | method B: R1 = |
| carbamoylphenyl)quinolin- | 1H), 8.13(s, 1H), 8.04- | Observed mass [M + H]: | n-butyl; |
| 2-yl]oxy]acetic acid | 8.01(m, 2H), 7.69-7.66 (m, | 379.00 | R2 = 3-amido- |
| | 3H), 7.51-7.40(m, 2H), | Exact mass: 378.16 | phenyl; R3 = |
| | 7.39(s, 1H), 6.92 (s, 1H), | | ethyl acid |
| | 4.63 (s, 2H), 2.63(t, J = 8 Hz, | | |
| | 2H), 1.53-1.45 (m, 2H), 1.30- | | |
| | 1.26(m, 2H), 0.85 (t, J = | | |
| | 7.2 Hz, 3H) | | |
| 2-{[4-(3- | 400 MHz-DMSO-d6: 12.5(s, | LCMS_5 min/2.85 | method A: R1 = |
| cyanophenyl)-6- | 1H), 8.01-7.98(m, 2H), 7.84 | Observed mass: | n-pentyl; |
| pentylquinolin-2- | (d, J = 7.6 Hz, 1H), 7.76 (t, J = | Exact mass: 387.19 | R2 = 3-cyano- |
| yl](methyl)amino}acetic | 7.6 Hz, 1H), 7.55(d, J = 8.4 Hz, | | phenyl; R3 = |
| acid | 1H), 7.40(dd, J = 8.8, 2.0 Hz, | | ethyl acid; |
| | 1H), 7.21 (s, 1H), 6.95 (s, | | R4 = methyl |
| | 1H), 4.32 (s, 2H), 3.18 (s, | | |
| | 3H), 2.56 (t, J = 7.6 Hz, 2H), | | |
| | 1.55-1.51(m, 2H), 1.30- | | |
| | 1.26(m, 4H), 0.83 (t, J = 8 Hz, | | |
| | 3H) | | |
| 2-{[4-(3- | 400 MHz-CDCl3: 12.5(s, 1H), | LCMS_5 min/2.59 | method A: R1 = |
| carbamoylphenyl)- | 7.97-7.94(m, 2H), 7.71(d, J = | Observed mass [M + H]: | n-pentyl; |
| 6-pentylquinolin-2- | 8 Hz, 1H), 7.65-7.63(m, 2H), | 406.26 | R2 = 3-amido- |
| yl](methyl)amino}acetic | 7.52-7.48 (m, 1H), 7.44 (s, | Exact mass: 405.21 | phenyl; R3 = |
| acid | 1H), 7.31 (s, 1H), 6.86 (s, | | ethyl acid; |
| | 1H), 4.34 (s, 2H), 3.30 (s, | | R4 = methyl |
| | 3H), 2.63 (t, J = 7.6 Hz, 2H), | | |
| | 1.57-1.51(m, 2H), 1.30- | | |
| | 1.26(m, 4H), 0.86 (t, J = | | |
| | 6.5 Hz, 3H) | | |
| 2-{[4-(3- | 400 MHz-DMSO-d6: 12.5(s, | RND-FA-3.5/1.875 | method A: R1 = |
| cyanophenyl)-6- | 1H), 8.01-7.98(m, 2H), | Observed mass [M + H]: | n-propyl; |
| propylquinolin-2- | 7.82(d, J = 7.6 Hz, 1H), 7.75 | 360.00 | R2 = 3-cyano- |
| yl](methyl)amino}acetic | (t, J = 7.6 Hz, 1H), 7.53(d, J = | Exact mass: 359.16 | phenyl; R3 = |
| acid | 9.2 Hz, 1H), 7.38(dd, J = 8.8, | | ethyl acid; |
| | 2.0 Hz, 1H), 7.19 (s, 1H), 6.88 | | R4 = methyl |
| | (s, 1H), 4.16 (s, 2H), 3.17 (s, | | |
| | 3H), 2.55 (t, J = 7.6 Hz, 2H), | | |
| | 1.57-1.51(m, 2H), 0.85 (t, J = | | |
| | 8 Hz, 3H) | | |
| 2-{[4-(3- | 400 MHz-DMSO-d6: 12.5(s, | LCMS_5 min/2.41 | method A: R1 = |
| carbamoylphenyl)- | 1H), 8.05(m, 1H), 8.01- | Observed mass [M + H]: | n-propyl; |
| 6-propylquinolin-2- | 7.99(m, 2H), 7.65-7.61(m, | 378.23 | R2 = 3-amido- |
| yl](methyl)amino}acetic | 2H), 7.55(d, J = 8 Hz, 1H), | Exact mass: 377.17 | phenyl; R3 = |
| acid | 7.44 (s, 1H), 7.39 (d, J = | | ethyl acid; |
| | 9.6 Hz, 1H), 7.26 (s, 1H), 6.92 | | R4 = methyl |
| | (s, 1H), 4.32 (s, 2H), 3.19 (s, | | |
| | 3H), 2.55 (m, 2H), 1.57- | | |
| | 1.51(m, 2H), 0.85 (t, J = | | |
| | 6.5 Hz, 3H) | | |
| 2-{[4-(3- | 400 MHz-DMSO-d6: 12.5(s, | RND-FA-3.5/1.765 | method A: R1 = |
| cyanophenyl)-6- | 1H), 8.01-7.98(m, 2H), | Observed mass [M + H]: | ethyl; |
| ethylquinolin-2- | 7.84(d, J = 7.6 Hz, 1H), 7.76 | 346.00 | R2 = 3-cyano- |
| yl](methyl)amino}acetic | (t, J = 7.6 Hz, 1H), 7.55(d, J = | Exact mass: 345.15 | phenyl; R3 = |
| acid | 9.2 Hz, 1H), 7.42(dd, J = 8.8, | | ethyl acid; |
| | 2.0 Hz, 1H), 7.22(s, 1H), 6.92 | | R4 = methyl |
| | (s, 1H), 4.24 (s, 2H), 3.19 (s, | | |
| | 3H), 2.61(dd, J = 15.2, 7.6 | | |
| | Hz, 2H), 1.14(t, J = 8 Hz, 3H) | | |
| 2-{[4-(3- | 400 MHz-DMSO-d6: 12.5(s, | Observed mass: | method A: R1 = |
| carbamoylphenyl)- | 1H), 8.08(m, 1H), 8.01- | Exact mass: 363.16 | ethyl; |
| 6-ethylquinolin-2- | 7.97(m, 2H), 7.65-7.61(m, | | R2 = 3-amido- |
| yl](methyl)amino}acetic | 2H), 7.52(d, J = 8 Hz, 1H), | | phenyl; R3 = |
| acid | 7.49 (s, 1H), 7.37 (d, J = | | ethyl acid; |
| | 9.6 Hz, 1H), 7.23 (s, 1H), 6.81 | | R4 = methyl |
| | (s, 1H), 4.20 (s, 2H), 3.16 (s, | | |
| | 3H), 2.57 (m, 2H), 1.53(m, | | |
| | 2H), 1.29-1.22(m, 2H), 0.85 | | |
| | (t, J = 6.5 Hz, 3H) | | |
| 2-{[6-bromo-4-(3- | 400 MHz-DMSO-d6: 12.5(s, | RND-FA-3.5/1.977 | method A: R1 = |
| cyanophenyl)quinolin-2- | 1H), 8.05(m, 1H), 8.03- | Observed mass [M + H]: | bromo; |
| yl](methyl)amino}acetic | 8.01(m, 1H), 7.87(d, J = | 395.90 | R2 = 3-cyano- |
| acid | 7.6 Hz, 1H), 7.78(t, J = 7.6 Hz, | Exact mass: 395.03 | phenyl; R3 = |
| | 1H), 7.66(dd, J = 8.8, 2.4 Hz, | | ethyl acid; |
| | 1H), 7.56(d, J = 8 Hz, 1H), | | R4 = methyl |
| | 7.51(d, J = 2 Hz, 1H), 7.11 (s, | | |
| | 1H), 6.81 (s, 1H), 4.44 (s, | | |
| | 2H), 3.21 (s, 3H) | | |
| 2-{[6-bromo-4-(3- | 400 MHz-DMSO-d6: 12.5(s, | LCMS_5 min/2.36 | method A: R1 = |
| carbamoylphenyl)quinolin-2- | 1H), 8.05(m, 1H), 8.03- | Observed mass [M − H]: | ethyl; |
| yl](methyl)amino}acetic | 7.98(m, 2H), 7.67-7.60(m, | 412.50 | R2 = 3-amido- |
| acid | 3H), 7.54-7.50(m, 3H), 6.92 | Exact mass: 413.04 | phenyl; R3 = |
| | (s, 1H), 4.44 (s, 2H), 3.18 (s, | | ethyl acid; |
| | 3H) | | R4 = methyl |
| 2-{[6-butyl-4-(3- | 400 MHz-DMSO-d6: 12.5(s, | RND-FA-3.5/1.985 | method A: R1 = |
| cyanophenyl)quinolin-2- | 1H), 8.01-7.97(m, 2H), | Observed mass [M + H]: | n-butyl; |
| yl](methyl)amino}acetic | 7.85(d, J = 7.6 Hz, 1H), | 374.00 | R2 = 3-cyano- |
| acid | 7.77(t, J = 8.4 Hz, 1H), 7.55(d, | Exact mass: 373.18 | phenyl; R3 = |
| | J = 8.8 Hz, 1H), 7.55(dd, J = | | ethyl acid; |
| | 8.8, 1.6 Hz, 1H), 7.22 (s, 1H), | | R4 = methyl |
| | 7.01 (s, 1H), 4.42 (s, 2H), | | |
| | 3.16 (s, 3H), 2.57 (t, J = 8 Hz, | | |
| | 2H), 1.55-1.47 (m, 2H), 1.36- | | |
| | 1.22(m, 2H), 0.86 (t, J = | | |
| | 6.5 Hz, 3H) | | |
| 2-{[6-butyl-4-(3- | 400 MHz-DMSO-d6: 12.5(s, | LCMS_5 min/2.51 | method A: R1 = |
| carbamoylphenyl)quinolin-2- | 1H), 8.08(m, 1H), 8.01- | Observed mass [M + H]: | n-butyl; |
| yl](methyl)amino}acetic | 7.97(m, 2H), 7.65-7.61(m, | 392.21 | R2 = 3-amidoo- |
| acid | 2H), 7.52(d, J = 8 Hz, 1H), | Exact mass: 391.19 | phenyl; R3 = |
| | 7.49 (s, 1H), 7.37 (d, J = | | ethyl acid; |
| | 9.6 Hz, 1H), 7.23 (s, 1H), 6.81 | | R4 = methyl |
| | (s, 1H), 4.20 (s, 2H), 3.16 (s, | | |
| | 3H), 2.57 (m, 2H), 1.53(m, | | |
| | 2H), 1.29-1.22(m, 2H), 0.85 | | |
| | (t, J = 6.5 Hz, 3H) | | |
| 2-{2-[(6-chloro-4- | | Observed mass: | |
| phenylquinolin-2- | | Exact mass: 383.10 | |
| yl)(methyl)amino]acetamido]acetic | | | |
| acid | | | |
| 2-[methyl(6-pentyl- | 500 MHz-DMSO-d6: 12.5(s, | RND-FA-3.5/2.088 | method A: R1 = |
| 4-phenylquinolin-2- | 1H), 7.57-7.49 (m, 6H), 7.39 | Observed mass [M + H]: | n-pentyl; |
| yl)amino]acetic | (dd, J = 6.8, 1.6 Hz, 1H), 7.31 | 363.10 | R2 = phenyl; |
| acid | (s, 1H), 6.87 (s, 1H), 4.34 (s, | Exact mass: 362.20 | R3 = ethyl |
| | 2H), 3.17 (s, 3H), 2.57 (t, J = | | acid; |
| | 7.5 Hz, 2H), 1.53(dd, J = | | R4 = methyl |
| | 14.8, 7.2 Hz, 2H), 1.29- | | |
| | 1.22(m, 4H), 0.83 (t, J = | | |
| | 6.5 Hz, 3H) | | |
| 2-[methyl(4-phenyl- | 400 MHz-DMSO-d6: 12.5(s, | RND-FA-3.5/1.880 | method A: R1 = |
| 6-propylquinolin-2- | 1H), 7.58-7.49 (m, 6H), 7.39 | Observed mass [M + H]: | n-propyl; |
| yl)amino] acetic | (d, J = 7.6 Hz, 1H), 7.32 (s, | 335.10 | R2 = phenyl; |
| acid | 1H), 6.88 (s, 1H), 4.35 (s, | Exact mass: 334.17 | R3 = ethyl |
| | 2H), 3.19 (s, 3H), 2.54 (dd, J = | | acid; |
| | 14.4, 7.2 Hz, 2H), 1.54(dd, | | R4 = methyl |
| | J = 14.8, 7.2 Hz, 2H), 0.85 (t, | | |
| | J = 6.8 Hz, 3 Hz) | | |
| 2-[(6-ethyl-4- | 400 MHz-DMSO-d6: 12.5(s, | RND-FA-3.5/1.780 | method A: R1 = |
| phenylquinolin-2- | 1H), 7.58-7.52 (m, 6H), 7.41 | Observed mass [M + H]: | ethyl; |
| yl)(methyl)amino]acetic | (d, J = 8.8 Hz, 1H), 7.34 (s, | 321.00 | R2 = phenyl; |
| acid | 1H), 6.88 (s, 1H), 4.35 (s, | Exact mass: 320.15 | R3 = ethyl |
| | 2H), 3.19 (s, 3H), 2.61 (dd, J = | | acid; |
| | 14.4, 7.2 Hz, 2H), 1.12 (t, | | R4 = methyl |
| | J = 6.8 Hz, 3 Hz) | | |
| 1-(6-hexyl-4- | 500 MHz-DMSO-d6: 12.5(s, | RND-FA-3.5/2.238 | method A: R1 = |
| phenylquinolin-2- | 1H), 8.28(br s, 1H), 7.58-7.48 | Observed mass [M + H]: | n-hexyl; |
| yl)pyrrolidine-2- | (m, 6H), 7.38 (d, J = 10 | 403.10 | R2 = phenyl; |
| carboxylic acid | Hz, 1H), 7.31 (s, 1H), 6.69 (s, | Exact mass: 402.23 | R3-R4 = 2- |
| | 1H), 4.55 (s, 1H), 3.70- | | carboxylic |
| | 3.55(m, 2H), 2.57 (t, J = | | acid proline; |
| | 9.0 Hz, 2H), 2.30-2.23(m, 1H), | | |
| | 2.18-2.00(m, 3H), 1.54- | | |
| | 1.50(m, 2H), 1.24-1.21(m, | | |
| | 6H), 0.82 (t, J = 6.8 Hz, 3 Hz) | | |
| 6-hexyl-4-phenyl-2- | 400 MHz-CDCl3: 7.68(d, J = | RND-FA-3.5/2.238 | method A: R1 = |
| (piperidin-1- | 8.8 Hz, 1H), 7.50-7.45(m, 5H), | Observed mass [M + H]: | n-hexyl; |
| yl)quinoline | 7.38-7.34 (m, 2H), 6.88 (br s, | 373.10 | R2 = phenyl; |
| | 1H), 3.71(s, 4H), 2.60 (t, J = | Exact mass: 372.26 | R3-R4 = |
| | 7.2 Hz, 2H), 1.68(m, 6H), | | Piperidinyl |
| | 1.58-1.54(m, 2H), 1.35- | | |
| | 1.24(m, 6H), 0.85 (t, J = | | |
| | 6.8 Hz, 3 Hz) | | |
| 2-[(6-hexyl-4- | 400 MHz-DMSO-d6: 8.3(s, | RND-FA-3.5/2.197 | method A: R1 = |
| phenylquinolin-2- | 1H), 7.57-7.47 (m, 6H), | Observed mass [M + H]: | n-hexyl; |
| yl)(methyl)amino]acetic | 7.38(dd, J = 8.4, 1.2 Hz, 1H), | 377.10 | R2 = phenyl; |
| acid | 7.30(s, 1H), 6.84 (br s, 1H), | Exact mass: 376.22 | R3 = ethyl |
| | 4.27 (s, 2H), 3.18 (s, 3H), | | acid; |
| | 2.57 (t, J = 7.2 Hz, 2H), 1.55- | | R4 = methyl |
| | 1.49(m, 2H), 1.30-1.22(m, | | |
| | 6H), 0.82 (t, J = 6.8 Hz, 3 Hz) | | |
| 1-(6-butyl-4- | 400 MHz-DMSO-d6: 12.5(s, | RND-FA-3.5/2.001 | method A: R1 = |
| phenylquinolin-2- | 1H), 7.58-7.47 (m, 6H), 7.38 | Observed mass [M + H]: | n-butyl; |
| yl)pyrrolidine-2- | (d, J = 8.8 Hz, 1H), 7.31(s, | 375.10 | R2 = phenyl; |
| carboxylic acid | 1H), 6.69 (br s, 1H), 4.55 (s, | Exact mass: 374.20 | R3-R4 = 2- |
| | 2H), 3.68-3.55(m, 2H), 2.57 | | carboxylic |
| | (t, J = 7.2 Hz, 2H), 2.24- | | acid proline; |
| | 2.20(m, 1H), 2.08-1.97(m, | | |
| | 3H), 1.55-1.49(m, 2H), 1.30- | | |
| | 1.22(m, 2H), 0.86 (t, J = | | |
| | 6.8 Hz, 3 Hz) | | |
| 6-butyl-4-phenyl-2- | 400 MHz-CDCl3: 7.68(d, J = | RND-FA-3.5/2.074 | method A: R1 = |
| (piperidin-1- | 8.8 Hz, 1H), 7.50-7.45(m, 5H), | Observed mass [M + H]: | n-butyl; |
| yl)quinoline | 7.38-7.34 (m, 2H), 6.88 (br s, | 345.10 | R2 = phenyl; |
| | 1H), 3.72(s, 4H), 2.61 (t, J = | Exact mass: 344.23 | R3-R4 = |
| | 7.2 Hz, 2H), 1.60-1.52 (m, | | Piperidinyl |
| | 8H), 1.35-1.24(m, 2H), 0.89 | | |
| | (t, J = 6.8 Hz, 3 Hz) | | |
| 2-[(6-bromo-4- | 400 MHz-DMSO-d6: 13.2 (s, | RND-FA-3.5/2.129 | method B: R1 = |
| phenylquinolin-2- | 1H), 7.79(dd, J = 8.8, 2.4 Hz, | Observed mass [M + H]: | bromo; |
| yl)oxy]acetic acid | 1H), 7.62-7.56 (m, 3H), 7.54- | 357.90 | R2 = phenyl; |
| | 7.48(m, 4H), 6.61 (s, 1H), | Exact mass: 357.00 | R3 = ethyl acid |
| | 5.05 (s, 2H) | | |
| 2-[(6-bromo-4- | 400 MHz-DMSO-d6: 12.6 (s, | RND-FA-3.5/1.96 | method A: R1 = |
| phenylquinolin-2- | 1H), 7.66-7.53 (m, 8H), 7.02 | Observed mass [M + H]: | bromo; |
| yl)(methyl)amino]acetic | (br s, 1H), 4.44 (s, 2H), 3.21 | 370.90 | R2 = phenyl; |
| acid | (s, 3H) | Exact mass: 370.03 | R3 = ethyl |
| | | | acid; |
| | | | R4 = methyl |
| 2-[(6-pentyl-4- | 400 MHz-DMSO-d6: 13.1 (s, | Observed mass [M + H]: | method B: R1 = |
| phenylquinolin-2- | 1H), 7.59-7.54 (m, 3H), 7.57- | 350.00 | n-pentyl; |
| yl)oxy]acetic acid | 7.47 (m, 3H), 7.39 (d, J = 8.8 | Exact mass: 349.17 | R2 = phenyl; |
| | Hz, 1H), 7.23 (d, J = 1.6 Hz, | | R3 = ethyl acid |
| | 1H), 6.51 (br s, 1H), 5.03 (s, | | |
| | 2H), 2.55 (m, 2H), 1.53-1.46 | | |
| | (m, 2H), 1.28-1.19 (m, 4H), | | |
| | 0.82 (t, J = 6.8 Hz, 3 Hz) | | |
| 2-[(4-phenyl-6- | 400 MHz-DMSO-d6: 13.1 (s, | RND-FA-3.5/2.24 | method B: R1 = |
| propylquinolin-2- | 1H), 7.57-7.53 (m, 3H), 7.49- | Observed mass [M + H]: | n-propyl; |
| yl)oxy]acetic acid | 7.42 (m, 3H), 7.31 (d, J = 8.8 | 322.10 | R2 = phenyl; |
| | Hz, 1H), 7.20 (d, J = 1.6 Hz, | Exact mass: 321.14 | R3 = ethyl acid |
| | 1H), 6.47 (br s, 1H), 4.80 (s, | | |
| | 2H), 2.50 (dd, J = 14.8, | | |
| | 7.8 Hz, 2H), 1.50 (m, 2H), | | |
| | 0.84 (t, J = 6.8 Hz, 3 Hz) | | |
| 2-[(6-ethyl-4- | 400 MHz-DMSO-d6: 13.1 (s, | ANL-MCL3/2.17 | method B: R1 = |
| phenylquinolin-2- | 1H), 7.60-7.48 (m, 6H), 7.41 | Observed mass [M + H]: | ethyl; |
| yl)oxy]acetic acid | (d, J = 8.0 Hz, 1H), 7.20 (d, | 308.30 | R2 = phenyl; |
| | J = 1.2 Hz, 1H), 6.52 (br s, 1H), | Exact mass: 307.12 | R3 = ethyl acid |
| | 5.06 (s,2H), 2.59 (dd, J = | | |
| | 14.8, 7.8 Hz, 2H), 1.13 (t, J = | | |
| | 6.8 Hz, 3 Hz) | | |
| 2-[(6-chloro-4- | 400 MHz-DMSO-d6: 13.4 (s, | RND-FA-3.5/2.084 | method B: R1 = |
| phenylquinolin-2- | 1H), 7.68(dd, J = 8.8, 2.4 Hz, | Observed mass [M + H]: | chloro; |
| yl)oxy]acetic acid | 1H), 7.62-7.50 (m, 6H), 7.34 | 314.00 | R2 = phenyl; |
| | (d, J = 2.4 Hz, 1H), 6.63 (s, | Exact mass: 313.05 | R3 = ethyl acid |
| | 1H), 5.06 (s, 2H), 3.19 (s, | | |
| | 3H), 2.53 (t, J = 7.2 Hz, 2H), | | |
| | 1.49-1.43(m, 2H), 1.29-1.24(m, | | |
| | 2H), 0.84 (t, J = 6.8 Hz, 3 Hz) | | |
| 2-[(6-butyl-4- | 400 MHz-DMSO-d6: 12.5 (s, | ANL-MCL3/2.413 | method B: R1 = |
| phenylquinolin-2- | 1H), 7.59-7.51 (m, 3H), 7.48- | Observed mass [M + H]: | n-butyl; |
| yl)oxy] acetic acid | 7.42 (m, 3H), 7.30 (d, J = 8.0 | 336.32 | R2 = phenyl; |
| | Hz, 1H), 7.20 (d, J = 1.2 Hz, | Exact mass: 335.15 | R3 = ethyl acid |
| | 1H), 6.47 (br s, 1H), 4.80 (s, | | |
| | 2H), 3.19 (s, 3H), 2.53 (t, J = | | |
| | 7.2 Hz, 2H), 1.49-1.43(m, 2H), | | |
| | 1.29-1.24(m, 2H), 0.84 (t, J = | | |
| | 6.8 Hz, 3 Hz) | | |
| 2-[(6-butyl-4- | 400 MHz-DMSO-d6: 12.5 (s, | RND-FA-3.5/1.993 | method A: R1 = |
| phenylquinolin-2- | 1H), 7.59-7.51 (m, 6H), 7.41 | Observed mass [M + H]: | n-butyl; |
| yl)(methyl)amino]acetic | (dd, J = 8.8, 2.0 Hz, 1H), 7.30 | 349.10 | R2 = phenyl; |
| acid | (d, J = 1.2 Hz, 1H), 6.91 (br s, | Exact mass: 348.18 | R3 = ethyl |
| | 1H), 4.42 (s, 2H), 3.19 (s, | | acid; |
| | 3H), 2.58 (t, J = 7.2 Hz, 2H), | | R4 = methyl |
| | 1.55-1.47(m, 2H), 1.29- | | |
| | 1.24(m, 2H), 0.86 (t, J = | | |
| | 6.8 Hz, 3 Hz) | | |
| 1-(6-chloro-4- | 500 MHz-CDCl3: 7.71 (d, J = | Observed mass: | method A: R1 = |
| phenylquinolin-2- | 9 Hz, 1H), 7.63(d, J = 2.5 Hz, | Exact mass: 352.10 | chloro; |
| yl)pyrrolidine-2- | 1H), 7.57-7.53(m, 4H), 7.47- | | R2 = phenyl; |
| carboxylic acid | 7.45(m, 2H), 6.93 (br s, 1H), | | R3-R4 = 2- |
| | 5.02(q, J = 7 Hz, 1H), 3.15 (s, | | carboxylic |
| | 3H), 1.59(d, J = 7 Hz, 3H), | | acid-proline |
| 2-[(6-chloro-4- | 500 MHz-CDCl3: 7.71 (d, J = | RND-X-bridge-5.0 MIN/ | method A: R1 = |
| phenylquinolin-2- | 9 Hz, 1H), 7.63(d, J = 2.5 Hz, | 2.306 | n-butyl; |
| yl)(methyl)amino]propanoic | 1H), 7.57-7.53(m, 4H), 7.47- | Observed mass [M + H]: | R2 = phenyl; |
| acid | 7.45(m, 2H), 6.93 (br s, 1H), | 341.10 | R3 = 1-methyl- |
| | 5.02(q, J = 7 Hz, 1H), 3.15 (s, | Exact mass: 340.10 | ethyl acid; |
| | 3H), 1.59(d, J = 7 Hz, 3H), | | R4 = methyl |
| 2-[(6-chloro-4- | 400 MHz-DMSO-d6: 12.5 (s, | RND-FA-3.5 MIN/ | method A: R1 = |
| phenylquinolin-2- | 1H), 7.63-7.51 (m, 7H), 7.45 | 1.865 | chloro; |
| yl)(methyl)amino]acetic | (d, J = 2.4 Hz, 1H), 7.02 (br s, | Observed mass [M + H]: | R2 = phenyl; |
| acid | 1H), 4.42 (s, 2H), 3.21 (s, 3H) | 327.00 | R3 = ethyl |
| | | Exact mass: 326.08 | acid; |
| | | | R4 = methyl |
| 6-chloro-4-phenyl- | | Observed mass: | method A: R1 = |
| 2-(piperidin-1- | | Exact mass: 322.12 | chloro; |
| yl)quinoline | | | R2 = phenyl; |
| | | | R3-R4 = |
| | | | piperidinyl |

### Antiviral Activity

Activity of compounds affecting viral replication and disease was tested against coronavirus replication in differentiated 3T3-L1 mouse adipocyte cells using human coronavirus OC43 as a model virus. 3T3-L1 preadipocytes (ATCC) were routinely cultured in a growth medium composed of DMEM high-glucose (Sigma), 10% FBS (Gibco), 10 U/ml penicillin and 10 μg/ml streptomycin (P/S; Gibco) in a standard cell culture incubator at 37° C. To induce adipogenic differentiation, a confluent layer of 3T3-L1 cells were incubated with the growth medium containing 2 μM rosiglitazone, 1 μM dexamethasone, 500 μM IBMX, and 1 μg/ml insulin (Sigma). Forty-eight (48) hours later (on day 2) and on days 4 and 6, medium of the cells was replaced with fresh medium containing 1 μg/ml insulin. On days 8 and 10, the medium was refreshed with regular growth medium and addition of insulin was omitted. On day 11 or 12, the cells were infected with human coronavirus OC43 (ATCC) at a multiplicity of 0.05 TCID50 (tissue-culture infectious-dose 50) per cell in DMEM containing 2% heat-inactivated FBS and P/S. One hour after infection, medium of the cells were replaced with fresh medium containing either the indicated FABP4 modulating compounds or the vehicle in which the compounds were dissolved (DMSO). The final compound concentration was 10 μM and final concentration of DMSO was 0.1% (v/v). The cells were incubated at 35° C. during the course of infection and compound treatment. Twenty-four hours after infection, medium of the cells was collected, cleared by centrifugation at 2,000 g for 5 minutes, and frozen at −80 degrees C. until further processing. The viral titers in the samples were determined by TCID50 assay in MRCS cells (ATCC) using Reed-Munch method and median fold reduction of virus titers in compound treated cells compared to vehicle control was calculated.

Table 2 shown below depicts the antiviral activity of the FABP4 modulating compounds. The compounds were categorized based on the magnitude of their antiviral activity. The compounds reduced virus titers >100-fold, between 10- to 99-fold, and between 2- to 9-fold compared to vehicle control were represented as “A”, “B” and “C”, respectively.

TABLE 2

| | | Antiviral |
|---|---|---|
| Compound | IUPAC name | activity |
| 1 | 2-{[6-butyl-4-(4-methylphenyl)quinolin- | B |
| | 2-yl](methyl)amino}acetic acid | |
| 2 | 2-{[6-butyl-4-(4-fluorophenyl)quinolin-2- | B |
| | yl](methyl)amino}acetic acid | |
| 3 | 2-[methyl(6-octyl-4-phenylquinolin-2- | A |
| | yl)amino]acetic acid | |
| 4 | 2-{[4-(4-fluorophenoxy)-6-octylquinolin- | B |
| | 2-yl](methyl)amino}acetic acid | |
| 5 | 2-{methyl[6-octyl-4-(pyridin-3- | A |
| | yloxy)quinolin-2-yl]amino}acetic acid | |
| 6 | 3-{[6-butyl-4-(4-fluorophenyl)quinolin-2- | C |
| | yl](methyl)amino}-2-methylpropanoic | |
| | acid | |
| 7 | 2-{methyl[4-phenyl-6-(2- | B |
| | phenylethyl)quinolin-2-yl]amino}acetic | |
| | acid | |
| 8 | 6-hexyl-N-methyl-4-phenyl-N-[(2H- | A |
| | 1,2,3,4-tetrazol-5-yl)methyl]quinolin-2- | |
| | amine | |
| 9 | 2-[(6-heptyl-4-phenylquinolin-2- | A |
| | yl)(methyl)amino]acetic acid | |
| 10 | cis-2-(6-hexyl-4-phenylquinolin-2- | C |
| | yl)cyclopropane-1-carboxylic acid | |
| 11 | 1-(6-hexyl-4-phenylquinolin-2-yl)-3- | B |
| | methylpyrrolidine-3-carboxylic acid | |
| 12 | 2-{[6-butyl-4-(3-cyanophenyl)quinolin-2- | B |
| | yl]oxy}propanoic acid | |
| 13 | 2-[methyl({4-phenyl-6-[2-(quinolin-6- | C |
| | yl)ethyl]quinolin-2-yl})amino]acetic acid | |
| 14 | 2-[(6-hexyl-4-phenylquinolin-2- | A |
| | yl)(methyl)amino]acetic acid | |
| 15 | 2-({6-[2-(3-methoxyphenyl)ethyl]-4- | B |
| | phenylquinolin-2- | |
| | yl}(methyl)amino)acetic acid | |
| 16 | 2-[methyl({4-phenyl-6-[2-(pyrimidin-2- | C |
| | yl)ethyl]quinolin-2-yl})amino]acetic acid | |
| 17 | 2-{[4-(3-cyanophenyl)-6-propylquinolin- | C |
| | 2-yl](methyl)amino}acetic acid | |
| 18 | 2-[(7-hexyl-4-phenylquinolin-2- | B |
| | yl)(methyl)amino]acetic acid | |

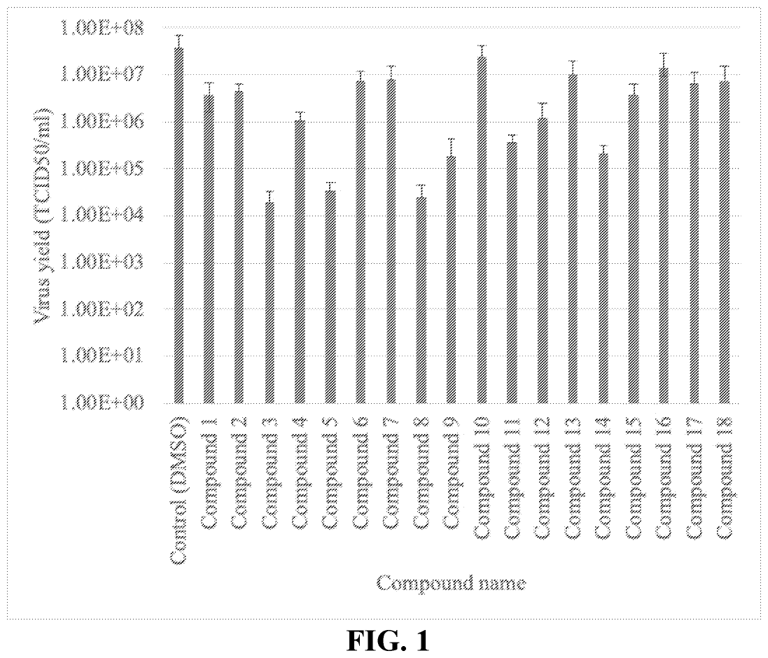

FIG. 1 depicts graphical charts showing the virus yield obtained from cells treated with the corresponding compounds and vehicle control (DMSO). More specifically, FIG. 1 includes a graph for the vehicle control (DMSO) and different FABP4 modulating compounds and include an x-axis associated with compound ID and a y-axis associated with a viral titer in TCID50/ml. Data are shown as mean (±standard deviation) of two or three biological replicates.

Although this invention has been described with a certain degree of particularity, it is to be understood that the present disclosure has been made only by way of illustration and that numerous changes in the details of construction and arrangement of parts may be resorted to without departing from the spirit and the scope of the invention.

What is claimed is:

1. A method for treating a viral disease in a subject comprising administering to said subject a therapeutically effective dose of one or more of the compounds described in Formula (I): 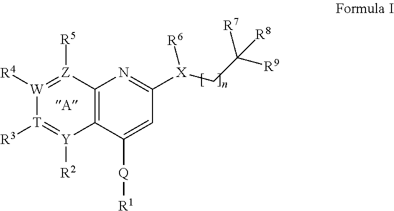
or pharmaceutically acceptable salts or esters thereof,
wherein R¹-R⁹ are independently hydrogen, CN, COOH, CONH₂, B(ORₐ)₂ (where Rₐ is H or alkyl), an acid isostere, halo, C₁-C₁₀ alkyl, C₁-C₁₀ alkenyl, C₁-C₁₀ alkynyl, aryl, aminoalkyl, haloalkyl, heteroaryl, cycloalkyl, or heterocycloalkyl;
wherein any of R²-R⁹ may be bonded with an adjoining R group thereby forming a fused cycloalkyl, fused heterocycloalkyl, fused aryl, or fused heteroaryl ring having from 4 to 10 carbon atoms;
wherein each of said C₁-C₁₀ alkyl, C₁-C₁₀ alkenyl, C₁-C₁₀ alkynyl, aryl, heteroaryl, cycloalkyl, or heterocycloalkyl is either unsubstituted or substituted with 1, 2, 3, 4 or 5 substituents which can be the same or different and are independently selected from the group consisting of hydrogen, deuterium, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, —C(O)alkyl; —C_{q}—U—C_{q}, where each q is independently 0 to 10 and U is any one of aryl, heteroaryl, cycloalkyl, heterocycloalkyl , O, S, SO₂, or N(R₁) (R₁), wherein each R₁ is independently hydrogen, C₁-C₁₀ alkyl, C₁-C₁₀ alkenyl, C₁-C₁₀ alkynyl, aryl, heteroaryl, cycloalkyl, or heterocycloalkyl is either unsubstituted or substituted with 1, 2, 3, 4 or 5 substituents which can be the same or different and are independently selected from the group consisting of hydrogen, deuterium, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, —C(O)alkyl;
Q is a bond or 0;
X is C, N, O, or S, with the proviso that R⁶ is not present if X is O or S;
wherein “A” is a saturated or unsaturated ring depicted by 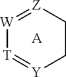
and Y, T, W, and Z are independently a bond, C, N, O, or alkyl or alkenyl having 1 to 4 carbon atoms; and
n is 0, 1, 2, or 3.

2. The compound of claim 1 wherein at least one of: R¹ and R², R² and R³, R³ and R⁴, R⁴ and R⁵, R⁵ and R⁶, R⁷ and R⁸ , or R⁸ and R⁹ are bonded forming a fused heteroaryl or fused heterocycloalkyl.

3. The compound of claim 1 wherein R³ is a Cₙ alkyl, and wherein the n is between 3-8.

4. The compound of claim 1 wherein R¹ is the halo.

5. The compound of claim 1 wherein Q comprises the bond.

6. A method for treating a viral disease in a subject comprising administering to said subject a therapeutically effective dose of one or more of the compounds described in Formula (II): 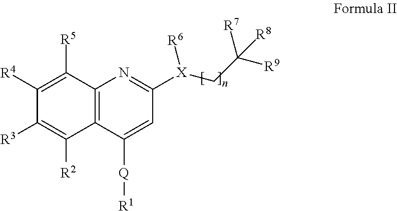
or pharmaceutically acceptable salts or esters thereof,
wherein R¹-R⁹ are independently hydrogen, CN, COOH, CONH₂, B(ORₐ)₂ (where Rₐ is H or alkyl), an acid isostere, halo, C₁-C₁₀ alkyl, C₁-C₁₀ alkenyl, C₁-C₁₀ alkynyl, aryl, aminoalkyl, haloalkyl, heteroaryl, cycloalkyl, or heterocycloalkyl,
wherein any of R²-R⁹ may be bonded with an adjoining R group thereby forming a fused cycloalkyl, fused heterocycloalkyl, fused aryl, or fused heteroaryl ring having from 4 to 10 carbon atoms and wherein each of said C₁-C₁₀ alkyl, C₁-C₁₀ alkenyl, C₁-C₁₀ alkynyl, aryl, heteroaryl, cycloalkyl, or heterocycloalkyl is either unsubstituted or substituted with 1, 2, 3, 4 or 5 substituents which can be the same or different and are independently selected from the group consisting of hydrogen, deuterium, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, —C(O)alkyl; —C_{q}—U—C_{q}, where each q is independently 0 to 10 and U is any one of aryl, heteroaryl, cycloalkyl, heterocycloalkyl, O, S, SO₂, or N(R₁)(R₁), wherein each R₁ is independently hydrogen, C₁-C₁₀ alkyl, C₁-C₁₀ alkenyl, C₁-C₁₀ alkynyl, aryl, heteroaryl, cycloalkyl, or heterocycloalkyl is either unsubstituted or substituted with 1, 2, 3, 4 or 5 substituents which can be the same or different and are independently selected from the group consisting of hydrogen, deuterium, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, —C(O)alkyl;
Q is a bond or O;
X is C, N, O, or S, with the proviso that R⁶ is not present if X is O or S;
X is C, N, O, or S; and
n is 0, 1, 2, or 3.

7. The compound of claim 6 wherein said heterocycloalkyl formed by bonding two of R⁷, R⁸ or R⁹ is

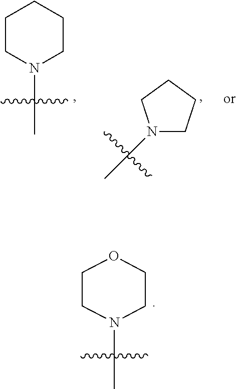 [SMILES: *N1CCCCC1 ; *N1CCCC1 ; *N1CCOCC1]

8. A method for treating a viral disease in a subject comprising administering to said subject a therapeutically effective dose of one or more of the compounds described in Formula (III): 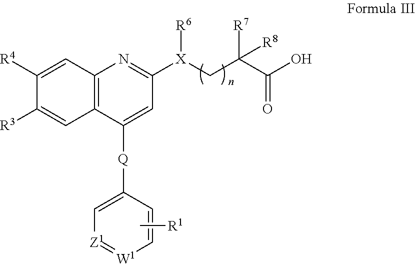
or pharmaceutically acceptable salts or esters thereof,
wherein R¹ is cyano, alkyl, hydrogen, halo, deuterium, amino, alkoxy, aminoalkyl, (amino)alkoxy, alkenyl, alkynyl, alkoxy, hydroxy, alkylhydroxy, aryloxy, alkyl(aryl), (alkoxyalkyl)amino, aryl, aryl(halo), heteroaryl, hydroxyl-alkyl, hydroxyl-aryl, (aryl)alkyl, C(O)OH, —S(O)₂-alkyl, —S(O)₂-aryl, —C(O)alkyl, or C(O)NH₂;
wherein R³ and R⁴ are independently halo or C₁-C₁₀ alkyl, —C_{q}—U—C_{q}, where each q is independently 0 to 10 and U is any one of O, S, SO₂, or N(R₁)(R₁), wherein each R₁ is independently hydrogen, C₁-C₁₀ alkyl, C₁-C₁₀ alkenyl, C₁-C₁₀ alkynyl, aryl, heteroaryl, cycloalkyl, or heterocycloalkyl is either unsubstituted or substituted with 1, 2, 3, 4 or 5 substituents which can be the same or different and are independently selected from the group consisting of hydrogen, deuterium, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, —C(O)alkyl;
wherein R⁶ is independently H or alkyl;
wherein R⁷ and R⁸ are independently hydrogen, deuterium, fluoro or alkyl;
wherein R⁶-R⁷ may be bonded with an adjoining R group thereby forming a fused cycloalkyl, fused heterocycloalkyl, fused aryl, or fused heteroaryl ring having from 4 to 10 carbon atoms and wherein each of said C₁-C₁₀ alkyl, C₁-C₁₀ alkenyl, C₁-C₁₀ alkynyl, aryl, heteroaryl, cycloalkyl, or heterocycloalkyl is either unsubstituted or substituted with 1, 2, 3, 4 or 5 substituents which can be the same or different and are independently selected from the group consisting of hydrogen, deuterium, halo, amino, alkoxy, cyano, aminoalkyl-, (amino)alkoxy-, -alkyl, -alkenyl, -alkynyl, alkoxy-, hydroxy, -alkylhydroxy, aryloxy-, -alkyl(aryl), (alkoxyalkyl)amino-, -aryl, -aryl(halo), -heteroaryl, hydroxyl-alkyl-, hydroxyl-aryl-, (aryl)alkyl-, —S(O)₂-alkyl, —S(O)₂-aryl, —C(O)alkyl
n is 0 or 1;
Q is a bond or O;
X is C, N, O, or S, with the proviso that R⁶ is not present if X is O or S; and
Z¹ or W¹ are independently C, N, O, or S.

9. The compound of claim 8 wherein X is N.

10. The compound of claim 8 wherein R⁶ is hydrogen and Xis N.

11. The compound of claim 8 wherein R³ is alkyl and R¹ is cyano.

12. The compound of claim 8 wherein X is N, and n=0.

13. The compound of claim 8 wherein X is N, and n=1.

14. The compounds of claim 8 wherein X is N, and Z¹ and W¹ are C.

15. A compound selected from the group consisting of: 3-{[6-butyl-4-(4-fluorophenyl)quinolin-2-yl](methyl)amino}-2-methylpropanoic acid, 3-{[4-(4-fluorophenyl)-6-hexylquinolin-2-yl](methyl)amino}-2-methylpropanoic acid, 2-{[4-(4-fluorophenyl)-6-pentylquinolin-2-yl](methyl)amino}acetic acid, 2-{[4-(4-fluorophenyl)-6-hexylquinolin-2-yl](methyl)amino}acetic acid, 2-{[6-hexyl-3-methyl-4-(morpholin-4-yl)quinolin-2-yl](methyl)amino}acetic acid, 2-{[4-(4-fluorophenyl)-6-hexyl-3-methylquinolin-2-yl](methyl)amino}acetic acid, 2{[4,6-bis(4-fluorophenyl)quinolin-2-yl](methyl)amino}acetic acid, 2-{[4-(4-fluorophenyl)-6-hexylquinolin-2-yl](2-methylpropyl)amino}acetic acid, 2-{[4-(4-fluorophenyl)-6-hexylquinolin-2-yl](propyl)amino}acetic acid, 2-{[4-(4-fluorophenyl)-6-hexylquinolin-2-yl]amino}acetic acid, 2-{ethyl[4-(4-fluorophenyl)-6-hexylquinolin-2-yl]amino}acetic acid, 2-{[6-hexyl-4-(pyridin-3-yloxy)quinolin-2-yl](methyl)amino}acetic acid, 2-{[6-hexyl-4-(pyridin-4-yloxy)quinolin-2-yl](methyl)amino} acetic acid, 2-{[4-(3-fluorophenoxy)-6-hexylquinolin-2-yl](methyl)amino}acetic acid, 2-{[4-(4-fluorophenoxy)-6-hexylquinolin-2-yl](methyl)amino}acetic acid, 2-{[4-(4-fluorophenyl)-6-octylquinolin-2-yl](2-methylpropyl)amino}acetic acid, 2-{[4-(4-fluorophenyl)-6-octylquinolin-2-yl](propyl)amino}acetic acid, 2-{ethyl[4-(4-fluorophenyl)-6-octylquinolin-2-yl]amino}acetic acid, 2-{methyl[6-octyl-4-(pyridin-3-yloxy)quinolin-2-yl]amino}acetic acid, 2-{methyl[6-octyl-4-(pyridin-4-yloxy)quinolin-2-yl]amino}acetic acid, 2-{[4-(3-fluorophenoxy)-6-octylquinolin-2-yl](methyl)amino}acetic acid, 2-{[4-(4-fluorophenoxy)-6-octylquinolin-2-yl](methyl)amino}acetic acid, 2-{[6-decyl-4-(4-fluorophenyl)quinolin-2-yl](methyl)amino}acetic acid, 2-{[4-(4-fluorophenyl)-6-heptylquinolin-2-yl](methyl)amino}acetic acid, 2-{[4-(4-fluorophenyl)-6-octylquinolin-2-yl](methyl)amino}acetic acid, 2-[(6-hexylquinolin-2-yl)(methyl)amino]acetic acid, 2-{2-[(carboxymethyl)(methyl)amino]-6-hexylquinolin-4-yl}benzoic acid, 2-{[4-(4,4-difluoropiperidin-1-yl)-6-hexylquinolin-2-yl](methyl)amino}acetic acid, 2-{[4-(3,-difluoropyrrolidin-1-yl)-6-hexylquinolin-2-yl](methyl)amino}acetic acid, 2-{[6-hexyl-4-(morpholin-4-yl)quinolin-2-yl](methyl)amino}acetic acid, 2-{[6-butyl-4-(2-methyl-pyridin-4-yl)quinolin-2-yl](methyl)amino}acetic acid, 2{[4-(3,5-dimethyl-1,2-oxazol-4-yl)-6-hexylquinolin-2-yl](methyl)amino}acetic acid, 2{[4-(3-cyanophenyl)-6-hexylquinolin-2-yl](methyl)amino}acetic acid, 3-{[4-(3-cyanophenyl)-6-hexylquinolin-2-yl](methyl)amino}butanoic acid, 3-[(6-hexyl-4-phenylquinolin-2-yl)(methyl)amino]-2-methylpropanoic acid, 2-[methyl(6-pentanamido-4-phenylquinolin-2-yl)amino]acetic acid, 2-{methyl[6-(pentyloxy)-4-phenylquinolin-2-yl]amino}acetic acid, 2-[(7-bromo-4-phenylquinolin-2-yl)(methyl)amino] acetic acid, 2-[(7-hexyl-4-phenylquinolin-2-yl)(methyl)amino]acetic acid, 2-[methyl(6-octyl-4-phenylquinolin-2-yl)amino]acetic acid, 3-{[6-hexyl-4-(pyridin-3-yl)quinolin-2-yl](methyl)amino}-2-methylpropanoic acid, 2-{[6-hexyl-4-(pyridin-3-yl)quinolin-2-yl](methyl)amino}acetic acid, 2{[4-(3-cyanophenyl)-6-hexylquinolin-2-yl]oxy}acetic acid, 3-{[4-(3-cyanophenyl)-6-hexylquinolin-2-yl](methyl)amino}-2-methylpropanoic acid, 2-{[4-(3-cyanophenyl)-6-hexylquinolin-2-yl](methyl)amino}acetic acid, 1-[6-hexyl-4-(pyridin-3-yl)quinolin-2-yl]piperidine-3-carboxylic acid, 1-(6-hexyl-4-phenylquinolin-2-yl)piperidine-3-carboxylic acid, 2-{[6-butyl-4-(4-hydroxyphenyl)quinolin-2-yl](methyl)amino}acetic acid, 2-{[6-butyl-4-(3-hydroxyphenyl)quinolin-2-yl](methyl)amino} acetic acid, 2-{[6-butyl-4-(2-hydroxyphenyl)quinolin-2-yl](methyl)amino}acetic acid, 2-{[6-butyl-4-(4-fluorophenyl)quinolin-2-yl](methyl)amino}acetic acid, 2-{[6-butyl-4-(3-fluorophenyl)quinolin-2-yl](methyl)amino}acetic acid, 2-{[6-butyl-4-(2-fluorophenyl)quinolin-2-yl](methyl)amino}acetic acid, 2-{[6-butyl-4-(4-methylphenyl)quinolin-2-yl](methyl)amino}acetic acid, 2-{[6-butyl-4-(3-methylphenyl)quinolin-2-yl](methyl)amino}acetic acid, 2-{[6-butyl-4-(2-methylphenyl)quinolin-2-yl](methyl)amino}acetic acid, 2-{[6-butyl-4-(4-cyanophenyl)quinolin-2-yl](methyl)amino}acetic acid, 2-{[6-butyl-4-(4-carbamoylphenyl)quinolin-2-yl](methyl)amino}acetic acid, 2-{[6-butyl-4-(pyridin-4-yl)quinolin-2-yl](methyl)amino}acetic acid, 6-butyl-2-(carboxymethoxy)-4-phenylquinoline-3-carboxylic acid, 2-{[6-butyl-4-(pyridin-3-yl)quinolin-2-yl](methyl)amino}acetic acid, 1-[6-butyl-4-(3-cyanophenyl)quinolin-2-yl]piperidine-3-carboxylic acid, 4-(6-butyl-4-phenylquinolin-2-yl)morpholine-2-carboxylic acid, 1-(6-butyl-4-phenylquinolin-2-yl)piperidine-3-carboxylic acid, 3-{[6-butyl-4-(3-cyanophenyl)quinolin-2-yl](methyl)amino}-2-methylpropanoic acid, 3-{[6-butyl-4-(pyridin-3-yl)quinolin-2-yl](methyl)amino}-2-methylpropanoic acid, 3-[(6-butyl-4-phenylquinolin-2-yl)(methyl)amino]-2-methylpropanoic acid, 3-[(6-butyl-4-phenylquinolin-2-yl)(methyl)amino]butanoic acid, 3-[(6-butyl-4-phenylquinolin-2-yl)(methyl)amino]propanoic acid, N-(6-butyl-4-(3-cyanophenyl)quinolin-2-yl)-N-methylvaline, 2-{[4-(3-cyanophenyl)-6-pentylquinolin-2-yl]oxy}acetic acid, 2-{[4-(3-carbamoylphenyl)-6-pentylquinolin-2-yl]oxy}acetic acid, 2-{[4-(3-cyanophenyl)-6-propylquinolin-2-yl]oxy}acetic acid, 2-{[4-(3-carbamoylphenyl)-6-propylquinolin-2-yl]oxy}acetic acid, 2-{[4-(3-carbamoylphenyl)-6-ethylquinolin-2-yl]oxy}acetic acid, 2{[6-bromo-4-(3-cyanophenyl)quinolin-2-yl]oxy}acetic acid, 2-{[6-bromo-4-(3-carbamoylphenyl)quinolin-2-yl]oxy}acetic acid, 2-{[6-butyl-4-(3-cyanophenyl)quinolin-2-yl]oxy}acetic acid, 2-{[6-butyl-4-(3-carbamoylphenyl)quinolin-2-yl]oxy}acetic acid, 2-{[4-(3-cyanophenyl)-6-pentylquinolin-2-yl](methyl)amino}acetic acid, 2-{[4-(3-carbamoylphenyl)-6-pentylquinolin-2-yl](methyl)amino}acetic acid, 2-{[4-(3-cyanophenyl)-6-propylquinolin-2-yl](methyl)amino}acetic acid, 2-{[4-(3-carbamoylphenyl)-6-propylquinolin-2-yl](methyl)amino}acetic acid, 2-{[4-(3-cyanophenyl)-6-ethylquinolin-2-yl](methyl)amino}acetic acid, 2-{[4-(3-carbamoylphenyl)-6-ethylquinolin-2-yl](methyl)amino}acetic acid, 2-{[6-bromo-4-(3-cyanophenyl)quinolin-2-yl](methyl)amino}acetic acid, 2-{[6-bromo-4-(3-carbamoylphenyl)quinolin-2-yl](methyl)amino}acetic acid, 2-{[6-butyl-4-(3-cyanophenyl)quinolin-2-yl](methyl)amino}acetic acid, 2-{[6-butyl-4-(3-carbamoylphenyl)quinolin-2-yl](methyl)amino}acetic acid, 2-{2-[(6-chloro-4-phenylquinolin-2-yl)(methyl)amino]acetamido}acetic acid, 2-[methyl(6-pentyl-4-phenylquinolin-2-yl)amino]acetic acid, 2-[methyl(4-phenyl-6-propylquinolin-2-yl)amino]acetic acid, 2-[(6-ethyl-4-phenylquinolin-2-yl)(methyl)amino]acetic acid, 1-(6-hexyl-4-phenylquinolin-2-yl)pyrrolidine-2-carboxylic acid, 6-hexyl-4-phenyl-2-(piperidin-1-yl)quinoline, 2-[(6-hexyl-4-phenylquinolin-2-yl)(methyl)amino]acetic acid, 1-(6-butyl-4-phenylquinolin-2-yl)pyrrolidine-2-carboxylic acid, 6-butyl-4-phenyl-2-(piperidin-1-yl)quinoline, 2-[(6-bromo-4-phenylquinolin-2-yl)oxy]acetic acid, 2-[(6-bromo-4-phenylquinolin-2-yl)(methyl)amino]acetic acid, 2-[(6-pentyl-4-phenylquinolin-2-yl)oxy]acetic acid, 2-[(4-phenyl-6-propylquinolin-2-yl)oxy]acetic acid, 2-[(6-ethyl-4-phenylquinolin-2-yl)oxy]acetic acid, 2-[(6-chloro-4-phenylquinolin-2-yl)oxy]acetic acid, 2-[(6-butyl-4-phenylquinolin-2-yl)oxy]acetic acid, 2-[(6-butyl-4-phenylquinolin-2-yl)(methyl)amino]acetic acid, 1-(6-chloro-4-phenylquinolin-2-yl)pyrrolidine-2-carboxylic acid, 2-[(6-chloro-4-phenylquinolin-2-yl)(methyl)amino]propanoic acid, 2-[(6-chloro-4-phenylquinolin-2-yl)(methyl)amino]acetic acid, 6-chloro-4-phenyl-2-(piperidin-1-yl)quinoline, 3-{[6-butyl-4-(4-fluorophenyl)quinolin-2-yl](methyl)amino}-2-methylpropanoic acid, 2-[methyl(6-octyl-4-phenylquinolin-2-yl)amino]acetic acid, 2-{[6-butyl-4-(4-fluorophenyl)quinolin-2-yl](methyl)amino}acetic acid, 2-{methyl[6-octyl-4-(pyridin-3-yloxy)quinolin-2-yl]amino}acetic acid, 2-{[6-butyl-4-(3-fluorophenyl)quinolin-2-yl](methyl)amino}acetic acid, 2-[(6-hexyl-4-phenylquinolin-2-yl)(methyl)amino]acetic acid, 2-{[4-(4-fluorophenoxy)-6-octylquinolin-2-yl](methyl)amino} acetic acid, 2-{[6-butyl-4-(3-methylphenyl)quinolin-2-yl](methyl)amino}acetic acid, 2-[(6-butyl-4-phenylquinolin-2-yl)(methyl)amino]acetic acid, 2-{[6-decyl-4-(4-fluorophenyl)quinolin-2-yl](methyl)amino}acetic acid, 2-[methyl(4-phenyl-6-propylquinolin-2-yl)amino]acetic acid, 2-{[6-butyl-4-(4-rnethylphenyl)quinolin-2-yl](methyl)amino}acetic acid, 3-[(6-butyl-4-phenylquinolin-2-yl)(methyl)amino]-2-methylpropanoic acid, 2{[4-(4-fluorophenyl)-6-pentylquinolin-2-yl](methyl)amino}acetic acid, 2-{[6-butyl-4-(3-cyanophenyl)quinolin-2-yl](methyl)amino}acetic acid, 2-{[6-butyl-4-(2-fluorophenyl)quinolin-2-yl](methyl)amino}acetic acid, 2-{[6-hexyl-4-(pyridin-3-yloxy)quinolin-2-yl](methyl)amino}acetic acid, 2{[4-(3-fluorophenoxy)-6-octylquinolin-2-yl](methyl)amino}acetic acid, 2-[methyl(6-pentyl-4-phenylquinolin-2-yl)amino]acetic acid, 3-[(6-hexyl-4-phenylquinolin-2-yl)(methyl)amino]-2-methylpropanoic acid, 2{[4-(3-cyanophenyl)-6-propylquinolin-2-yl](methyl)amino}acetic acid, 2-{[6-hexyl-4-(morpholin-4-yl)quinolin-2-yl](methyl)amino}acetic acid, 3-{[4-(4-fluorophenyl)-6-hexylquinolin-2-yl](methyl)amino}-2-methylpropanoic acid, 2{[4-(4-fluorophenyl)-6-hexylquinolin-2-yl](methyl)amino}acetic acid, 3-{[4-(3-cyanophenyl)-6-hexylquinolin-2-yl](methyl)amino}-2-methylpropanoic acid, 2-[methyl({6-[2-(4-methylphenyl)ethyl]-4-phenylquinolin-2-yl})amino]acetic acid, 2-[methyl({6-[2-(3-methylphenyl)ethyl]-4-phenylquinolin-2-yl})amino]acetic acid, 6-hexyl-N-methyl-4-phenyl-N-[(2H-1,2,3,4-tetrazol-5-yl)methyl]quinolin-2-amine, 2-{methyl[4-phenyl-6-(2-phenylethyl)quinolin-2-yl]amino}acetic acid, 2-({6-[2-(3-chlorophenyl)ethyl]-4-phenylquinolin-2-yl}(methyl)amino)acetic acid, 5-{[4-phenyl-6-(2-phenylethyl)quinolin-2-yl]methyl }-1,3-thiazolidine-2,4-dione, 2-({6-[2-(4-chlorophenyl)ethyl]-4-phenylquinolin-2-yl}(methyl)amino)acetic acid, 2-[methyl({4-phenyl-6-[2-(pyridin-3-yl)ethyl]quinolin-2-yl})amino]acetic acid, 2-[methyl({4-phenyl-6-[2-(quinolin-6-yl)ethyl]quinolin-2-yl})amino]acetic acid, 2-[(6-heptyl-4-phenylquinolin-2-yl)(methyl)amino]acetic acid, 2-[methyl({6-[2-(2-methylphenyl)ethyl]-4-phenylquinolin-2-yl})amino]acetic acid, 2-[methyl({4-phenyl-6-[2-(pyridin-2-yl)ethyl]quinolin-2-yl})amino]acetic acid, cis-2-(6-hexyl-4-phenylquinolin-2-yl)cyclopropane-1-carboxylic acid, 1-(6-hexyl-4-phenylquinolin-2-yl)-3-methylpyrrolidine-3-carboxylic acid, 2-[methyl({4-phenyl-6-[2-(pyrimidin-2-yl)ethyl]quinolin-2-yl})amino]acetic acid, 2-[(6-hexyl-4-phenyl-5,6,7,8-tetrahydroquinolin-2-yl)(methyl)amino]acetic acid, 2-[methyl({4-phenyl-6-[2-(quinoxalin-6-yl)ethyl]quinolin-2-yl})amino]acetic acid, 5-[(6-hexyl-4-phenylquinolin-2-yl)methyl]-1,3-thiazolidine-2,4-dione, 2-({6-[(1E)-hex-1-en-1-yl]-4-phenylquinolin-2-yl}(methyl)amino)acetic acid, 2-{methyl[4-phenyl-6-(3-phenylpropyl)quinolin-2-yl]amino}acetic acid, 2-[methyl({4-phenyl-6-[2-(1,2,3,4-tetrahydroquinolin-6-yl)ethyl]quinolin-2-yl})amino]acetic acid, 2-({6-[2-(3-methoxyphenyl)ethyl]-4-phenylquinolin-2-yl}(methyl)amino)acetic acid, 2-({6-[2-(1,3-benzothiazol-2-yl)ethyl]-4-phenylquinolin-2-yl}(methyl)amino)acetic acid, 2-{[6-hexyl-4-(pyridin-4-yl)quinolin-2-yl]oxy}propanoic acid, and 3-(6-hexyl-4-phenylquinolin-2-yl)butanoic acid.

16. A method of treating and preventing an acute viral infection, the method comprising administering to a mammal an effective amount of a compound of claim 1.

17. The method of claim 16, wherein the acute viral infection is an acute respiratory viral infection.

18. The method of claim 17, wherein the acute respiratory viral infection is COVID-19 or severe acute respiratory syndrome (SARS) disease.

19. A method according to claim 16, wherein the subject is a human.

20. The compound according to claim 1 for use in the prophylaxis or treatment of COVID-19 or severe acute respiratory syndrome (SARS) disease.

21. A pharmaceutical composition comprising a compound according to claim 1 as the active ingredient.

22. The pharmaceutical composition according claim 21, further comprising at least one additional active ingredient or a pharmaceutically acceptable carrier.

23. A method for treating and preventing coronavirus or severe acute respiratory syndrome (SARS) disease, the method comprising: administering to a subject in need of such treatment an effective amount of a compound according to claim 1.

24. A method according to claim 23, wherein the subject is a human.
